(19) United States
(12) Patent Application Publication
(10) Pub. No.: US 2008/0194529 A1
(43) Pub. Date: Aug. 14, 2008

(54) HIGHLY SELECTIVE and LONG-ACTING PDE5 MODULATORS

(75) Inventors: Thomas G. Gant, Carlsbad, CA (US); Sepehr Sarshar, Cardiff by the Sea, CA (US)

Correspondence Address: GLOBAL PATENT GROUP - APX;Ms. LaVern Hall, 10411 Clayton Road, Suite 304, ST. LOUIS, MO, 63131, US

(73) Assignee: AUSPEX PHARMACEUTICALS, INC., Vista, CA (US)

(21) Appl. No.: 12/029,633

(22) Filed: Feb. 12, 2008

Related U.S. Application Data
(60) Provisional application No. 60/889,505, filed on Feb. 12, 2007.

(51) Int. Cl. A61K 31/573; C07D 487/04; A61K 31/519; A61P 15/10; A61P 9/12

(52) U.S. Cl. 514/171; 544/262; 514/262.1

(57) ABSTRACT

Disclosed herein are substituted phosphodiesterase type 5 enzyme modulators of Formula I, processes of preparation thereof, pharmaceutical compositions thereof, and methods of use thereof.

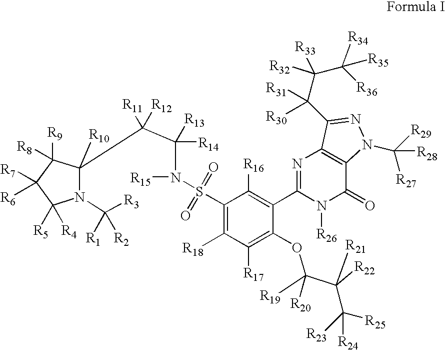

### CROSS REFERENCE TO RELATED APPLICATION

This application claims the benefit of priority of U.S. provisional application No. 60/889,505, filed Feb. 12, 2007 the disclosure of which is hereby incorporated by reference as if written herein in their entirety.

### FIELD

The present invention is directed to modulators of a phosphodiesterase type 5 (PDE5) enzyme and pharmaceutically acceptable salts and prodrugs thereof, the chemical synthesis thereof, and the medical use of such compounds for the treatment and/or management of hypertension, erectile dysfunction, and/or the inability to maintain improved erectile function.

### BACKGROUND

Udenafil (Zydena®) is a therapeutic agent hypothesized to improve erectile function endpoints through interaction with the phosphodiesterase type 5 (PDE5) enzyme. As such, udenafil belongs to the class of such agents that includes tadalafil (Clalis®), sildenafil (Viagra®), and vardenafil (Levitra®). These agents are purported to promote erectile response through inhibition of PDE5, the predominant PDE within the penis, which leads to higher intracellular levels of cyclic guanylate cyclase (cGMP). cGMP is a second messenger for the smooth-muscle relaxing effects of nitric oxide within the penis. The various agents differ in pharmacology primarily based on 1) onset and duration of action and 2) selectivity profiles vs. other PDEs. All three marketed agents have proven remarkably safe. These agents should not be taken by patients with unstable cardiovascular disease. Udenafil has been shown to exhibit greater selectivity against the known PDE homologues, than other PDE5 inhibitors. Udenafil is comparable to tadalafil in many respects, such as duration of action and high selectivity for PDE6, but udenafil has greater selectivity for PDE11 than tadalafil. Tadalafil, with a half life of 17.5 hours, has a much longer duration of action and improved exercise tolerance than either sildenfail or vardenafil, which have half lives of 4-5 hours. Consequently, tadalafil is associated with less planning or pressure to have sexual intercourse after dosing. Dissociation of the sexual activity from the time of dosing is associated with higher rates of patient and partner satisfaction. In prospective, randomized crossover clinical studies, patients preferred tadalafil over sildenafil by margins ranging from 7:3 to 9:1. Sildenafil and vardenafil both modulate PDE6 at higher rate than tadalafil. PDE6 modulation has been associated with chromatopsia. The side effects of chromatopsia, such as sensitivity to light and blurred vision, are therefore higher in patients taking sildenafil or vardenafil, about 2-3%, than patients taking tadalafil, about <0.1%. Tadalafil is less selective than sildenafil and vardenafil for PDE5 and for PDE11a. Activity at PDE11a is suspected to have a causal relationship with myalgia and testicular toxicity. The selectivity profile for udenafil is similar to sildenafil, which should impart greater safety for this agent.

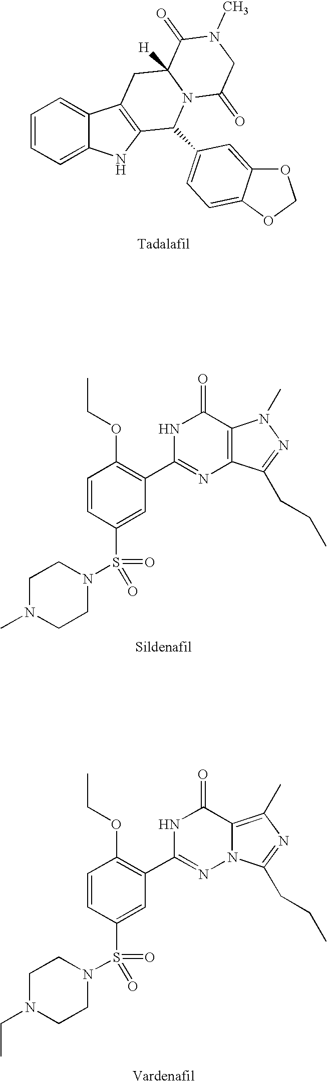

The benefits and shortcomings of these drugs have been reviewed. Some of these shortcomings can be traced to metabolism-related phenomena. Udenafil is converted in vivo by oxidative and conjugative degradation to multiple metabolites. Phase I metabolism leads to demethylation of the pyrazole, hydroxylation of the pyrazole propyl group, and dealkylation alpha to the sulfonamide nitrogen to afford an active metabolite. Because udenafil is metabolized primarily by cytochrome P₄₅₀ subtype 3A4 (CYP3A4), exposure to udenafil can influence polypharmacy. For example, CYP3A4 inhibitors such as HIV protease inhibitors, azole antifungals, and erythromycin can lead to higher than otherwise expected blood levels of udenafil. Conversely, co-administration of CYP3A4 inducers such as rifampin can decrease the otherwise expected blood levels of udenafil. Thus, the polypharmacy of udenafil is necessarily complex and has potential for adverse events. In addition, there may be increased inter-patient variability in response to polypharmacy. Analogs of udenafil as described herein have the potential to alleviate the problems associated with the commercially available PDE5 inhibitors while maintaining or improving efficacy. It is believed that the reduction in CYP3A4 clearance of udenafil analogs will be expected to increase the proportion of clearance via mechanisms less susceptible to polypharmaceutical complications. In addition, analogs of udenafil having an attenuated rate of oxidative metabolism will have an increased half-life, further augmenting their advantages vs. tadalafil, sildenafil and vardenafil. Potentially, a single dose of an udalafil analog, described herein, having an increased half-life may provide therapeutic coverage for an entire weekend or beyond while increasing safety parameters by reducing the likelihood of drug-drug interactions and by increasing safety as a result of the increased selectivity.

Disclosed herein is a compound having structural Formula I:

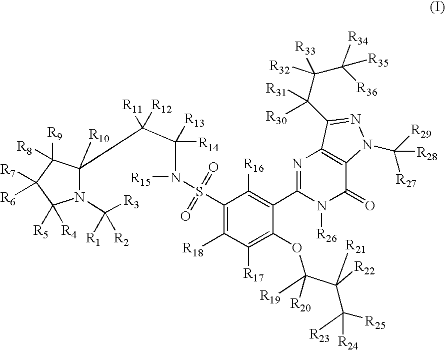

or a pharmaceutically acceptable salt, solvate, or prodrug thereof, wherein:

R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are independently selected from the group consisting of hydrogen, and deuterium; and

at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ is independently deuterium.

Also disclosed herein are pharmaceutical compositions comprising at least one compound as disclosed herein or a pharmaceutically acceptable salt, solvate, or prodrug thereof; in combination with one or more pharmaceutically acceptable excipients or carriers. Further, disclosed herein are methods of modulating the activity of a phosphodiesterase type 5 enzyme.

Further, disclosed herein is a method for treating, preventing, or ameliorating one or more of the following conditions including, but not limited to, hypertension, erectile dysfunction, the inability to maintain improved erectile function and/or any disorder which can be ameliorated, lessen, and/or attenuated by administering a phosphodiesterase type 5 enzyme modulator, which comprises administering to a subject a therapeutically effective amount of at least one compound as disclosed herein or a pharmaceutically acceptable salt, solvate, or prodrug thereof.

Also disclosed herein are articles of manufacture and kits containing compounds as disclosed herein. By way of example only a kit or article of manufacture can include a container (such as a bottle) with a desired amount of at least one compound (or pharmaceutical composition of a compound) as disclosed herein. Further, such a kit or article of manufacture can further include instructions for using said compound (or pharmaceutical composition of a compound) as disclosed herein. The instructions can be attached to the container, or can be included in a package (such as a box or a plastic or foil bag) holding the container.

In addition, disclosed herein are methods of treating a subject having, suspected of having, or being prone to a disorder, such as hypertension, erectile dysfunction, the inability to maintain improved erectile function, and/or any disorder which can be ameliorated, lessen, and/or attenuated by administering a phosphodiesterase type 5 enzyme modulator.

In another aspect are processes for preparing a compound as disclosed herein or other pharmaceutically acceptable derivative thereof such as a salt, solvate, or prodrug, as a PDE5 enzyme modulator.

Also disclosed herein are processes for formulating pharmaceutical compositions with a compound disclosed herein.

In certain embodiments said pharmaceutical composition is suitable for oral, parenteral, or intravenous infusion administration.

In yet other embodiments said pharmaceutical composition comprises a tablet, or capsule.

In certain embodiments the compounds as disclosed herein are administered in a dose of 0.5 milligram to 1000 milligram.

In yet further embodiments said pharmaceutical compositions further comprise another therapeutic agent.

In other embodiments said therapeutic agent is selected from the group consisting of: phosphodiesterase 5 inhibitors, experimental erectile dysfunction treatments, CYP3A inhibitors, CYP3A inducers, protease inhibitors, anti-fugal agents, antibacterials, antimycobacterial agents, sepsis treatments, steroidal drugs, anticoagulants, thrombolytics, non-steroidal anti-inflammatory agents, antiplatelet agents, endothelin converting enzyme (ECE) inhibitors, thromboxane enzyme antagonists, potassium channel openers, thrombin inhibitors, growth factor inhibitors, platelet activating factor (PAF) antagonists, anti-platelet agents, Factor VIIa Inhibitors and Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-PDE5 agents, beta-PDE5 agents, antiarrhythmic agents, diuretics, anti-diabetic agents, PPAR-gamma agonists, mineralocorticoid enzyme antagonists, aP2 inhibitors, protein tyrosine kinase inhibitors, antiinflammatories, antiproliferatives, chemotherapeutic agents, immunosuppressants, anticancer agents and cytotoxic agents, antimetabolites, farnesyl-protein transferase inhibitors, hormonal agents, microtubule-disruptor agents, microtubule-stabilizing agents, topoisomerase inhibitors, prenyl-protein transferase inhibitors and cyclosporins, TNF-alpha inhibitors, cyclooxygenase-2 (COX-2) inhibitors, gold compounds, and platinum coordination complexes.

In yet further embodiments said therapeutic agent is a phosphodiesterase 5 inhibitor.

In other embodiments said therapeutic agent is an experimental erectile dysfunction treatment.

In certain embodiments, the present invention discloses a method of treating a subject suffering from a PDE5-mediated disorder comprising administering to said subject a therapeutically effective amount of a compound as disclosed herein.

In other embodiments said PDE5-mediated disorder can be ameliorated by administering a PDE5 enzyme modulator.

In yet further embodiments said disorder is selected from the group consisting of hypertension, erectile dysfunction, and the inability to maintain improved erectile function.

In other embodiments the said wherein said disorder is erectile dysfunction.

In other embodiments said compound has at least one of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In yet further embodiments said compound has at least two of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

In certain embodiments said compound has a decreased metabolism by at least one polymorphically-expressed cytochrome P₄₅₀ isoform in said subject per dosage unit thereof as compared to the non-isotopically enriched compound.

In other embodiments said cytochrome P₄₅₀ isoform is selected from the group consisting of CYP2C8, CYP2C9, CYP2C19, and CYP2D6.

In yet further embodiments said compound is characterized by decreased inhibition of at least one cytochrome P₄₅₀ or monoamine oxidase isoform in said subject per dosage unit thereof as compared to the non-isotopically enriched compound.

In certain embodiments said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

### INCORPORATION BY REFERENCE

All publications and references cited herein, including those in the background section, are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

### DETAILED DESCRIPTION

To facilitate understanding of the disclosure set forth herein, a number of terms are defined below. Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood in the art to which this disclosure belongs. In the event that there is a plurality of definitions for a term used herein, those in this section prevail unless stated otherwise.

As used herein, the singular forms “a,” “an,” and “the” may refer to plural articles unless specifically stated otherwise.

The term “subject” refers to an animal, including, but not limited to, a primate (e.g., human monkey, chimpanzee, gorilla, and the like), rodents (e.g., rats, mice, gerbils, hamsters, ferrets, and the like), lagomorphs, swine (e.g., pig, miniature pig), equine, canine, feline, avian, reptilian, amphibian and the like. The terms “subject” and “patient” are used interchangeably herein, for example, to a mammalian subject, such as a human patient.

The terms “treat,” “treating,” and “treatment” are meant to include alleviating or abrogating a disorder; or one or more of the symptoms associated with the disorder; or alleviating or eradicating the cause(s) of the disorder itself.

The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ or the symbol “D,” when used to represent a given position in a drawing of a molecular structure, means means that the specified position is enriched with deuterium above the naturally occurring distribution of deuterium. In an embodiment deuterium enrichment is of no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

The terms “substantially pure” and “substantially homogeneous” mean sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), infrared spectroscopy (IR), gas chromatography (GC), Ultraviolet Spectroscopy (UV), nuclear magnetic resonance (NMR), atomic force spectroscopy, and mass spectroscopy (MS); or sufficiently pure such that further purification would not detectably alter the physical and chemical properties, or biological and pharmacological properties, such as enzymatic and biological activities, of the substance. In certain embodiments, “substantially pure” or “substantially homogeneous” refers to a collection of molecules, wherein at least about 50%, at least about 70%, at least about 80%, at least about 90%, at least about 95%, at least about 98%, at least about 99%, or at least about 99.5% of the molecules are a single compound, including a racemic mixture or single stereoisomer thereof, as determined by standard analytical methods.

The term “about” or “approximately” means an acceptable error for a particular value as determined by one of ordinary skill in the art, which depends in part on how the value is measured or determined. In certain embodiments, “about” can mean 1 or more standard deviations.

The terms “active ingredient” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “disorder” as used herein is intended to be generally synonymous, and is used interchangeably with, the terms “disease,” “syndrome” and “condition” (as in medical condition), in that all reflect an abnormal condition of the body or of one of its parts that impairs normal functioning and is typically manifested by distinguishing signs and symptoms.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “PDE5-mediated disorder,” refers to a disorder that is characterized by abnormal PDE5 enzyme activity or normal PDE5 enzyme activity that, when that activity is modified, leads to the amelioration of other abnormal biological processes. A PDE5 enzyme-mediated disorder may be completely or partially mediated by modulation of the PDE5 enzyme activity. In particular, a PDE5-mediated disorder is one in which modulation of PDE5 enzyme activity results in some effect on the underlying disorder, e.g., modulating PDE5 enzyme activity results in some improvement in at least some of the patients being treated.

The term “PDE5 enzyme” refers to one of the five subtypes of the enzyme phosphodiesterase. PDE5 is found in various tissues, most prominently the corpus cavernosum and the retina. PDE5 is a cGMP acceptor and metabolizer.

The term “modulator”, “modulate” or “modulation” refers to the ability of a compound disclosed herein to alter the function of a PDE5 enzyme. A modulator may activate the activity of a PDE5 enzyme, may activate or inhibit the activity of a PDE5 enzyme depending on the concentration of the compound exposed to the PDE5 enzyme, or may inhibit the activity of a PDE5 enzyme. Such activation or inhibition may be contingent on the occurrence of a specific event, such as activation of a signal transduction pathway, and/or may be manifest only in particular cell types. The term “modulate” or “modulation” also refers to altering the function of a PDE5 enzyme by increasing or decreasing the probability that a complex forms between a PDE5 enzyme and a natural binding partner. A modulator may increase the probability that such a complex forms between the PDE5 enzyme and the natural binding partner, may increase or decrease the probability that a complex forms between the PDE5 enzyme and the natural binding partner depending on the concentration of the compound exposed to the PDE5 enzyme, and or may decrease the probability that a complex forms between the PDE5 enzyme and the natural binding partner.

The term “protecting group” or “removable protecting group” refers to a group which, when bound to a functionality, such as the oxygen atom of a hydroxyl or carboxyl group, or the nitrogen atom of an amino group, prevents reactions from occurring at that functional group, and which can be removed by a conventional chemical or enzymatic step to reestablish the functional group (Greene and Wuts, Protective Groups in Organic Synthesis, 3^{rd} Ed., John Wiley & Sons, New York, N.Y., 1999).

The term “halogen”, “halide” or “halo” includes fluorine, chlorine, bromine, and iodine.

The definition of “amino protecting group” includes but is not limited to:

a. 2-methylthioethyl, 2-methylsulfonylethyl, 2-(p-toluenesulfonyl)ethyl, [2-(1,3-dithianyl)]methyl, 4-methylthiophenyl, 2,4-dimethylthiophenyl, 2-phosphonioethyl, 1-methyl-1-(triphenylphosphonio)ethyl, 1,1-dimethyl-2-cyanoethyl, 2-dansylethyl, 2-(4-nitrophenyl)ethyl, 4-phenylacetoxybenzyl, 4-azidobenzyl, 4-azidomethoxybenzyl, m-chloro-p-acyloxybenzyl, p-(dihydroxyboryl)benzyl, 5-benzisoxazolylmethyl, 2-(trifluoromethyl)-6-chromonylmethyl, m-nitrophenyl, 3,5-dimethoxybenzyl, 1-methyl-1-(3,5-dimethoxyphenyl)ethyl, o-nitrobenzyl, α-methylnitropiperonyl, 3,4-dimethoxy-6-nitrobenzyl, N-benzenesulfenyl, N-o-nitrobenzenesulfenyl, N-2,4-dinitrobenzenesulfenyl, N-pentachlorobenzenesulfenyl. N-2-nitro-4-methoxybenzenesulfenyl, N-triphenylmethylsulfenyl, N-1-(2,2,2-trifluoro-1,1-diphenyl)ethylsulfenyl, N-3-nitro-2-pyridinesulfenyl, N-p-toluenesulfonyl, N-benzenesulfonyl, N-2,3,6-trimethyl-4-methoxybenzenesulfonyl, N-2,4,6-trimethoxybenzene-sulfonyl, N-2,6-dimethyl-4-methoxybenzenesulfonyl, N-pentamethylbenzenesulfonyl, N-2,3,5,6-tetramethyl-4-methoxybenzenesulfonyl and the like;

b. —C(O)OR₄₀, where R₄₀ is selected from the group consisting of alkyl, substituted alkyl, aryl and more specifically R₄₀=methyl, ethyl, 9-fluorenylmethyl, 9-(2-sulfo)fluorenylmethyl. 9-(2,7-dibromo)fluorenylmethyl, 17-tetrabenzo[a,c,g,i]fluorenylmethyl. 2-chloro-3-indenylmethyl, benz[f]inden-3-ylmethyl, 2,7-di-t-butyl-[9-(10,10-dioxo-10,10,10,10-tetrahydrothioxanthyl)]methyl, 1,1-dioxobenzo[b]thiophene-2-ylmethyl, 2,2,2-trichloroethyl, 2-trimethylsilylethyl, 2-phenylethyl, 1-(1-adamantyl)-1-methylethyl, 2-chloroethyl, 1,1-dimethyl-2-haloethyl, 1,1-dimethyl-2,2-dibromoethyl, 1,1-dimethyl-2,2,2-trichloroethyl, 1-methyl-1-(4-biphenylyl)ethyl, 1-(3,5-di-tert-butylphenyl)-1-methylethyl, 2-(2′-pyridyl)ethyl, 2-(4′-pyridyl)ethyl, 2,2-bis(4′-nitrophenyl)ethyl, N-(2-pivaloylamino)-1,1-dimethylethyl, 2-[(2-nitrophenyl)dithio]-1-phenylethyl, tert-butyl, 1-adamantyl, 2-adamantyl, Vinyl, allyl, 1-Isopropylallyl, cinnamyl. 4-nitrocinnamyl, 3-(3-pyridyl)prop-2-enyl, 8-quinolyl, N-Hydroxypiperidinyl, alkyldithio, benzyl, p-methoxybenzyl, p-nitrobenzyl, p-bromobenzyl. p-chlorobenzyl, 2,4-dichlorobenzyl, 4-methylsulfinylbenzyl, 9-anthrylmethyl, diphenylmethyl, tert-amyl, S-benzyl thiocarbamate, butynyl, p-cyanobenzyl, cyclobutyl, cyclohexyl, cyclopentyl, cyclopropylmethyl, p-decyloxybenzyl, diisopropylmethyl, 2,2-dimethoxycarbonylvinyl, o-(N,N′-dimethylcarboxamido)benzyl, 1,1-dimethyl-3-(N,N′-dimethylcarboxamido)propyl, 1,1-dimethylpropynyl, di(2-pyridyl)methyl, 2-furanylmethyl, 2-Iodoethyl, isobornyl, isobutyl, isonicotinyl, p-(p′-methoxyphenylazo)benzyl, 1-methylcyclobutyl, 1-methylcyclohexyl, 1-methyl-1-cyclopropylmethyl, 1-methyl-1-(p-phenylazophenyl)ethyl, 1-methyl-1-phenylethyl, 1-methyl-1-4′-pyridylethyl, phenyl, p-(phenylazo)benzyl, 2,4,6-trimethylphenyl, 4-(trimethylammonium)benzyl, 2,4,6-trimethylbenzyl and the like. Other examples of amino protecting groups are given in Greene and Wutts, Protective Groups in Organic Synthesis, 3^{rd} Ed., John Wiley & Sons, New York, N.Y., 1999, which is incorporated by reference herein in its entirety.

The terms “oxidant” or “oxidizing agent” refer to any reagent that will increase the oxidation state of an atom, such as for example, hydrogen, carbon, nitrogen, sulfur, phosphorus and the like in the starting material by either adding an oxygen to this atom or removing an electron from this atom and as such would be obvious to one of ordinary skill and knowledge in the art. The definition of “oxidant” includes but is not limited to: osmium tetroxide, ruthenium tetroxide, ruthenium trichloride, potassium permanganate, meta-chloroperbenzoic acid, hydrogen peroxide, dimethyl dioxirane and the like.

The term “reducing reagent” refers to any reagent that will decrease the oxidation state of an atom in the starting material by either adding a hydrogen to this atom, or adding an electron to this atom, or by removing an oxygen from this atom and as such would be obvious to one of ordinary skill and knowledge in the art. The definition of “reducing reagent” includes but is not limited to: borane-dimethyl sulfide complex, 9-borabicyclo[3.3.1.]nonane (9-BBN), catechol borane, lithium borohydride, lithium borodeuteride, sodium borohydride, sodium borodeuteride, sodium borohydride-methanol complex, potassium borohydride, sodium hydroxyborohydride, lithium triethylborohydride, lithium n-butylborohydride, sodium cyanoborohydride, sodium cyanoborodeuteride, calcium (II) borohydride, lithium aluminum hydride, lithium aluminum deuteride, diisobutylAluminum hydride, n-butyl-diisobutylaluminum hydride, sodium bis-methoxyethoxyaluminum hydride, triethoxysilane, diethoxymethylsilane, lithium hydride, lithium, sodium, hydrogen Ni/B, and the like. Certain acidic and Lewis acidic reagents enhance the activity of reducing reagents. Examples of such acidic reagents include: acetic acid, methanesulfonic acid, hydrochloric acid, and the like. Examples of such Lewis acidic reagents include: trimethoxyborane, triethoxyborane, aluminum trichloride, lithium chloride, vanadium trichloride, dicyclopentadienyl titanium dichloride, cesium fluoride, potassium fluoride, zinc (II) chloride, zinc (II) bromide, zinc (II) iodide, and the like.

The terms “alkyl” and “substituted alkyl” are interchangeable and include substituted, optionally substituted and unsubstituted C₁-C₁₀ straight chain saturated aliphatic hydrocarbon groups, substituted, optionally substituted and unsubstituted C₂-C₁₀ straight chain unsaturated aliphatic hydrocarbon groups, substituted, optionally substituted and unsubstituted C₂-C₁₀ branched saturated aliphatic hydrocarbon groups, substituted and unsubstituted C₂-C₁₀ branched unsaturated aliphatic hydrocarbon groups, substituted, optionally substituted and unsubstituted C₃-C₈ cyclic saturated aliphatic hydrocarbon groups, substituted, optionally substituted and unsubstituted C₅-C₈ cyclic unsaturated aliphatic hydrocarbon groups having the specified number of carbon atoms. For example, the definition of “alkyl” shall include but is not limited to: methyl (Me), trideuteromethyl (—CD₃), ethyl (Et), propyl (Pr), butyl (Bu), pentyl, hexyl, heptyl, octyl, nonyl, decyl, undecyl, ethenyl, propenyl, butenyl, penentyl, hexenyl, heptenyl, octenyl, nonenyl, decenyl, undecenyl, isopropyl (i-Pr), isobutyl (i-Bu), tert-butyl (t-Bu), sec-butyl (s-Bu), isopentyl, neopentyl, cyclopropyl, cyclobutyl, cyclopentyl, cyclohexyl, cycloheptyl, cyclooctyl, cyclopentenyl, cyclohexenyl, cycloheptenyl, cyclooctenyl, methylcyclopropyl, ethylcyclohexenyl, butenylcyclopentyl, adamantyl, norbornyl and the like. Alkyl substituents are independently selected from the group consisting of hydrogen, deuterium, halogen, —OH, —SH, —NH₂, —CN, —NO₂, ═O, ═CH₂, trihalomethyl, carbamoyl, arylC₀₋₁₀alkyl, heteroarylC₀₋₁₀alkyl, C₁₋₁₀alkyloxy, arylC₀₋₁₀alkyloxy, C₁₋₁₀alkylthio, arylC₀₋₁₀alkylthio, C₁₋₁₀alkylamino, arylC₀₋₁₀alkylamino, N-aryl-N—C₀₋₁₀alkylamino, C₁₋₁₀alkylcarbonyl, arylC₀₋₁₀alkylcarbonyl, C₁₋₁₀alkylcarboxy, arylC₀₋₁₀alkylcarboxy, C₁₋₁₀alkylcarbonylamino, arylC₀₋₁₀alkylcarbonylamino, tetrahydrofuryl, morpholinyl, piperazinyl, hydroxypyronyl, —C₀₋₁₀alkylCOOR₄₁ and —C₀₋₁₀alkylCONR₄₂R₄₃ wherein R₄₁, R₄₂ and R₄₃ are independently selected from the group consisting of hydrogen, deuterium, alkyl, aryl, or R₄₂ and R₄₃ are taken together with the nitrogen to which they are attached forming a saturated cyclic or unsaturated cyclic system containing 3 to 8 carbon atoms with at least one substituent as defined herein.

The term “aryl” represents an unsubstituted, mono-, or polysubstituted monocyclic, polycyclic, biaryl aromatic groups covalently attached at any ring position capable of forming a stable covalent bond, certain preferred points of attachment being apparent to those skilled in the art (e.g., 3-phenyl, 4-naphthyl and the like). The aryl substituents are independently selected from the group consisting of hydrogen, deuterium, halogen, —OH, —SH, —CN, —NO₂, trihalomethyl, hydroxypyronyl, C₁₋₁₀alkyl, arylC₀₋₁₀alkyl, C₀₋₁₀alkyloxyC₀₋₁₀alkyl, arylC₀₋₁₀alkyloxyC₀₋₁₀alkyl, C₀₋₁₀alkylthioC₀₋₁₀alkyl, arylC₀₋₁₀alkylthioC₀₋₁₀alkyl, C₀₋₁₀alkylaminoC₀₋₁₀alkyl, arylC₀₋₁₀alkylaminoC₀₋₁₀alkyl, N-aryl-N—C₀₋₁₀alkylaminoC₀₋₁₀alkyl, C₁₋₁₀alkylcarbonylC₀₋₁₀alkyl, arylC₀₋₁₀alkylcarbonylC₀₋₁₀alkyl, C₁₋₁₀alkylcarboxyC₀₋₁₀alkyl, arylC₀₋₁₀alkylcarboxyC₀₋₁₀alkyl, C₁₋₁₀alkylcarbonylaminoC₀₋₁₀alkyl, arylC₀₋₁₀alkylcarbonylaminoC₀₋₁₀alkyl, —C₀₋₁₀alkylCOOR₄₁, and —C₀₋₁₀alkylCONR₄₂R₄₃ wherein R₄₁, R₄₂ and R₄₃ are independently selected from the group consisting of hydrogen, deuterium, alkyl, aryl or R₄₂ and R₄₃ are taken together with the nitrogen to which they are attached forming a saturated cyclic or unsaturated cyclic system containing 3 to 8 carbon atoms with at least one substituent as defined above.

The definition of “aryl” includes but is not limited to phenyl, pentadeuterophenyl, biphenyl, naphthyl, dihydronaphthyl, tetrahydronaphthyl, indenyl, indanyl, azulenyl, anthryl, phenanthryl, fluorenyl, pyrenyl and the like.

In light of the purposes described in the present disclosure, all references to “alkyl” and “aryl” groups or any groups ordinarily containing C—H bonds may include partially or fully deuterated versions as required to affect the improvements outlined herein.

### Deuterium Kinetic Isotope Effect

In an attempt to eliminate foreign substances, such as therapeutic agents, from its circulation system, the animal body expresses various enzymes, such as the cytochrome P₄₅₀ enzymes or CYPs, esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}, where E_{act} is the activation energy, T is temperature, R is the molar gas constant, k is the rate constant for the reaction, and A (the frequency factor) is a constant specific to each reaction that depends on the probability that the molecules will collide with the correct orientation. The Arrhenius equation states that the fraction of molecules that have enough energy to overcome an energy barrier, that is, those with energy at least equal to the activation energy, depends exponentially on the ratio of the activation energy to thermal energy (RT), the average amount of thermal energy that molecules possess at a certain temperature.

The transition state in a reaction is a short lived state (on the order of 10-14 sec) along the reaction pathway during which the original bonds have stretched to their limit. By definition, the activation energy E_{act} for a reaction is the energy required to reach the transition state of that reaction. Reactions that involve multiple steps will necessarily have a number of transition states, and in these instances, the activation energy for the reaction is equal to the energy difference between the reactants and the most unstable transition state. Once the transition state is reached, the molecules can either revert, thus reforming the original reactants, or new bonds form giving rise to the products. This dichotomy is possible because both pathways, forward and reverse, result in the release of energy. A catalyst facilitates a reaction process by lowering the activation energy leading to a transition state. Enzymes are examples of biological catalysts that reduce the energy necessary to achieve a particular transition state.

A carbon-hydrogen bond is by nature a covalent chemical bond. Such a bond forms when two atoms of similar electronegativity share some of their valence electrons, thereby creating a force that holds the atoms together. This force or bond strength can be quantified and is expressed in units of energy, and as such, covalent bonds between various atoms can be classified according to how much energy must be applied to the bond in order to break the bond or separate the two atoms.

The bond strength is directly proportional to the absolute value of the ground-state vibrational energy of the bond. This vibrational energy, which is also known as the zero-point vibrational energy, depends on the mass of the atoms that form the bond. The absolute value of the zero-point vibrational energy increases as the mass of one or both of the atoms making the bond increases. Since deuterium (D) has twice the mass of hydrogen (H), it follows that a C-D bond is stronger than the corresponding C—H bond. Compounds with C-D bonds are frequently indefinitely stable in H₂O, and have been widely used for isotopic studies. If a C—H bond is broken during a rate-determining step in a chemical reaction (i.e. the step with the highest transition state energy), then substituting a deuterium for that hydrogen will cause a decrease in the reaction rate and the process will slow down. This phenomenon is known as the Deuterium Kinetic Isotope Effect (DKIE). The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon. Substitution of tritium for hydrogen results in yet a stronger bond than deuterium and gives numerically larger isotope effects

Discovered in 1932 by Urey, deuterium (D) is a stable and non-radioactive isotope of hydrogen. It was the first isotope to be separated from its element in pure form and has twice the mass of hydrogen, and makes up about 0.02% of the total mass of hydrogen (in this usage meaning all hydrogen isotopes) on earth. When two deuterium atoms bond with one oxygen, deuterium oxide (D₂O or “heavy water”) is formed. D₂O looks and tastes like H₂O, but has different physical properties. It boils at 101.41° C. and freezes at 3.79° C. Its heat capacity, heat of fusion, heat of vaporization, and entropy are all higher than H₂O. It is more viscous and has different solubilizing properties than H₂O.

When pure D₂O is given to rodents, it is readily absorbed and reaches an equilibrium level that is usually about eighty percent of the concentration of what was consumed. The quantity of deuterium required to induce toxicity is extremely high. When 0% to as much as 15% of the body water has been replaced by D₂O, animals are healthy but are unable to gain weight as fast as the control (untreated) group. When about 15% to about 20% of the body water has been replaced with D₂O, the animals become excitable. When about 20% to about 25% of the body water has been replaced with D₂O, the animals are so excitable that they go into frequent convulsions when stimulated. Skin lesions, ulcers on the paws and muzzles, and necrosis of the tails appear. The animals also become very aggressive; males becoming almost unmanageable. When about 30%, of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O Studies have also shown that the use of D₂O can delay the growth of cancer cells and enhance the cytotoxicity of certain antineoplastic agents.

Tritium (T) is a radioactive isotope of hydrogen, used in research, fusion reactors, neutron generators and radiopharmaceuticals. Mixing tritium with a phosphor provides a continuous light source, a technique that is commonly used in wristwatches, compasses, rifle sights and exit signs. It was discovered by Rutherford, Oliphant and Harteck in 1934, and is produced naturally in the upper atmosphere when cosmic rays react with H₂ molecules. Tritium is a hydrogen atom that has 2 neutrons in the nucleus and has an atomic weight close to 3. It occurs naturally in the environment in very low concentrations, most commonly found as T₂O, a colorless and odorless liquid. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level.

Deuteration of pharmaceuticals to improve pharmacokinetics (PK), pharmacodynamics (PD), and toxicity profiles, has been demonstrated previously with some classes of drugs. For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity. Such pitfalls are non-obvious and are not predictable a priori for any drug class.

### Deuterated Substituted PDE5 Modulators

Certain PDE5 modulators are known in the art and are shown herein. Udenafil (Zydena®, DA-8159) is one such compound. The carbon-hydrogen bonds of udenafil contain a naturally occurring distribution of hydrogen isotopes, namely ¹H or protium (about 99.9844%), 2H or deuterium (about 0.0156%), and ³H or tritium (in the range between about 0.5 and 67 tritium atoms per 10¹⁸ protium atoms). Increased levels of deuterium incorporation produce a detectable Kinetic Isotope Effect (KIE) that could affect the pharmacokinetic, pharmacologic and/or toxicologic parameters of such PDE5 modulators relative to compounds having naturally occurring levels of deuterium.

Aspects of the present invention disclosed herein describe a novel approach to designing and synthesizing new analogs of these PDE5 modulators through chemical modifications and derivations of the carbon-hydrogen bonds of the modulators and/or of the chemical precursors used to synthesize said modulators. Suitable modifications of certain carbon-hydrogen bonds into carbon-deuterium bonds may generate novel PDE5 modulators with unexpected and non-obvious improvements of pharmacological, pharmacokinetic and toxicological properties in comparison to the non-isotopically enriched PDE5 modulators.

Based on discoveries made in our laboratory, as well as considering the KIE literature, udenafil is likely metabolized, in humans, at various C—H bonds. For example, the pyrazolo-N-methyl C—H bonds of udenafil are now known to be sites of cytochrome P₄₅₀ metabolism. Similarly, the pyrazolopropyl C—H bonds are also susceptible to P₄₅₀ oxidation. The toxicities and pharmacologies of all of udenafil's metabolites are not known with certainty. Furthermore, because some polymorphically expressed CYPs may oxidize udenafil, the prevention of such interactions decreases interpatient variability, decreases drug-drug interactions, increases T_{1/2}, decreases the necessary Cₘₐₓ, and improves several other ADMET parameters.

The deuterated analogs of this invention have the potential to uniquely maintain the beneficial aspects of the non-isotopically enriched drugs while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions. These drugs also have strong potential to reduce the cost-of-goods (COG) owing to the ready availability of inexpensive sources of deuterated reagents combined with previously mentioned potential for lowering the therapeutic dose. The deuteration approach has strong potential to slow the metabolism through the genetically polymorphically expressed CYPs. The deuteration approach has strong potential to slow the metabolism through CYP3A4, an isoform that is subject to inhibition or induction by many drugs.

Various deuteration patterns can be used to a) reduce or eliminate unwanted metabolites, b) increase the half-life of the parent drug, c) decrease the number of doses needed to achieve a desired effect, d) decrease the amount of a dose needed to achieve a desired effect, e) increase the formation of active metabolites, if any are formed, and/or f) decrease the production of deleterious metabolites in specific tissues and/or create a more effective drug and/or a safer drug for polypharmacy, whether the polypharmacy be intentional or not. The deuteration approach has strong potential to shunt clearance of such drugs through more universal pathways thus giving rise to more predictable ADMET responses throughout the dose range (which would also be lower via this invention) and decrease interpatient variability.

In one embodiment, disclosed herein is a compound having structural Formula I:

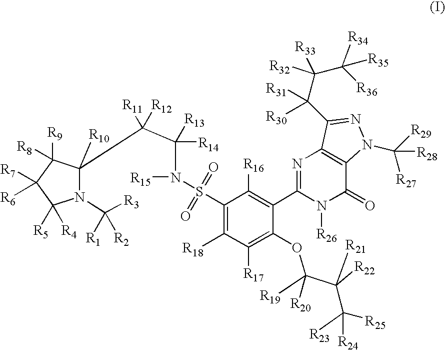

or a pharmaceutically acceptable salt, solvate, or prodrug thereof, wherein:

R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are independently selected from the group consisting of hydrogen, and deuterium; and

at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ is independently deuterium.

In a further embodiment, said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

In another embodiment, at least one R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ independently has deuterium enrichment of no less than about 1%, no less than about 5%, no less than about 10%, no less than about 20%, no less than about 50%, no less than about 70%, no less than about 80%, no less than about 90%, or no less than about 98%.

In yet another embodiment, a compound is selected from the group consisting of:

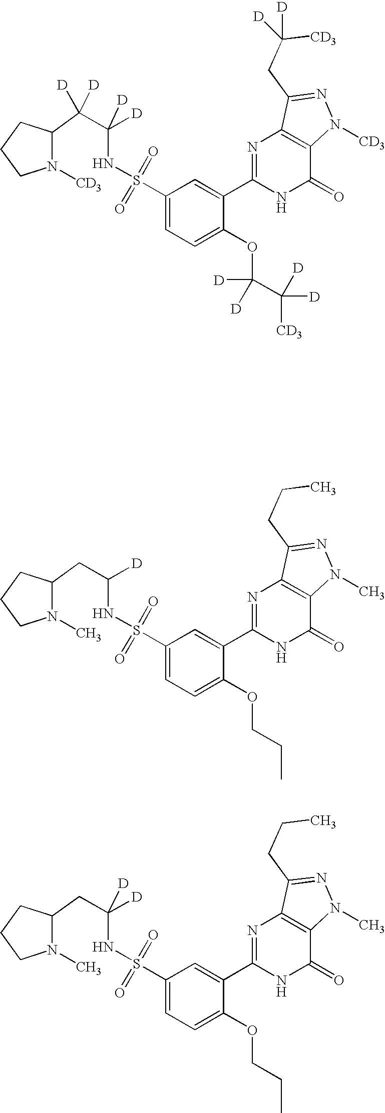

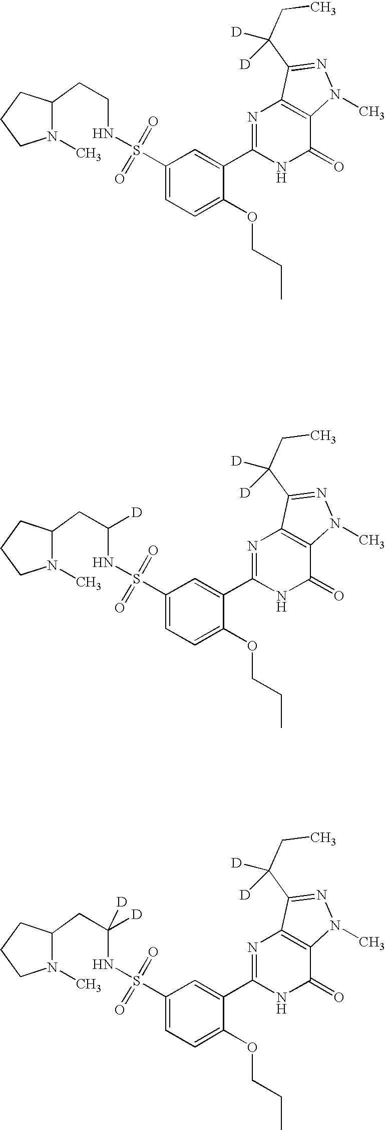

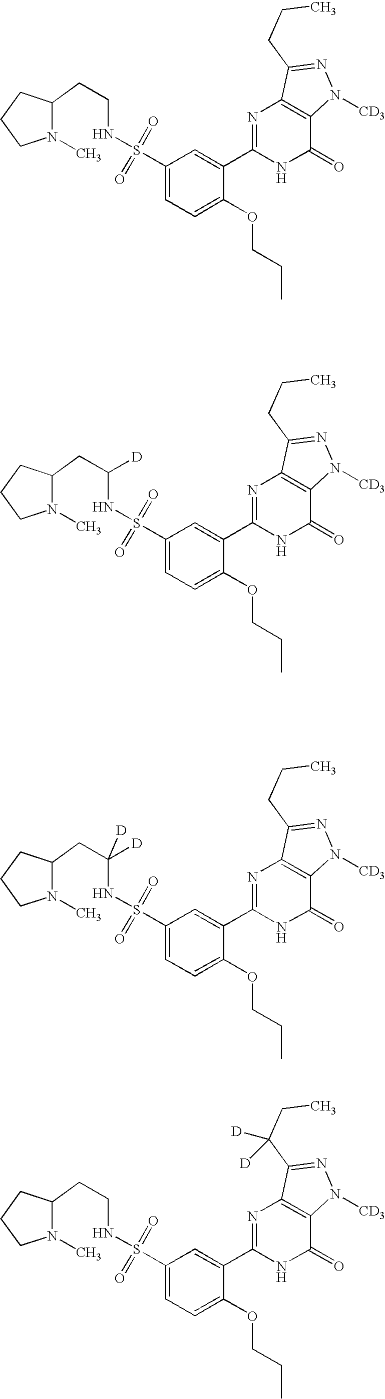

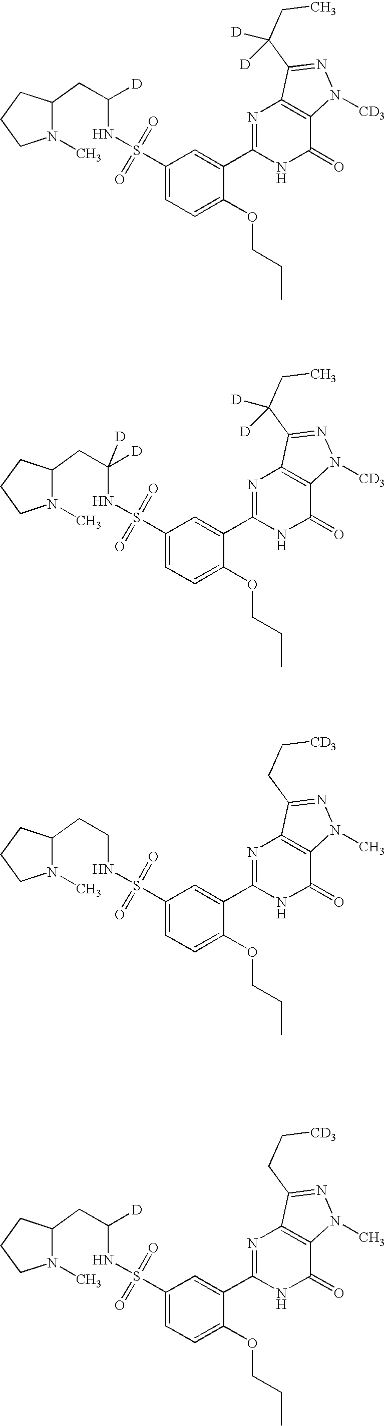

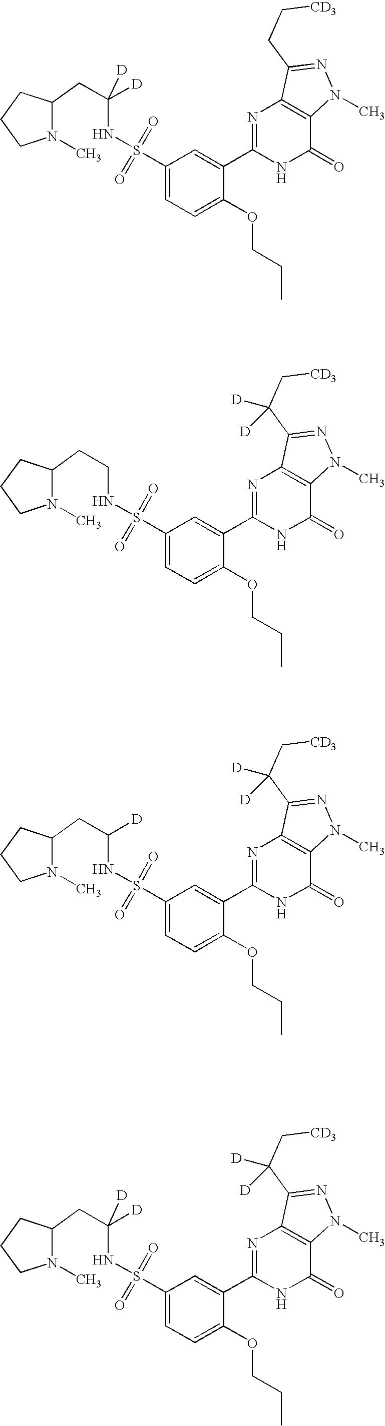

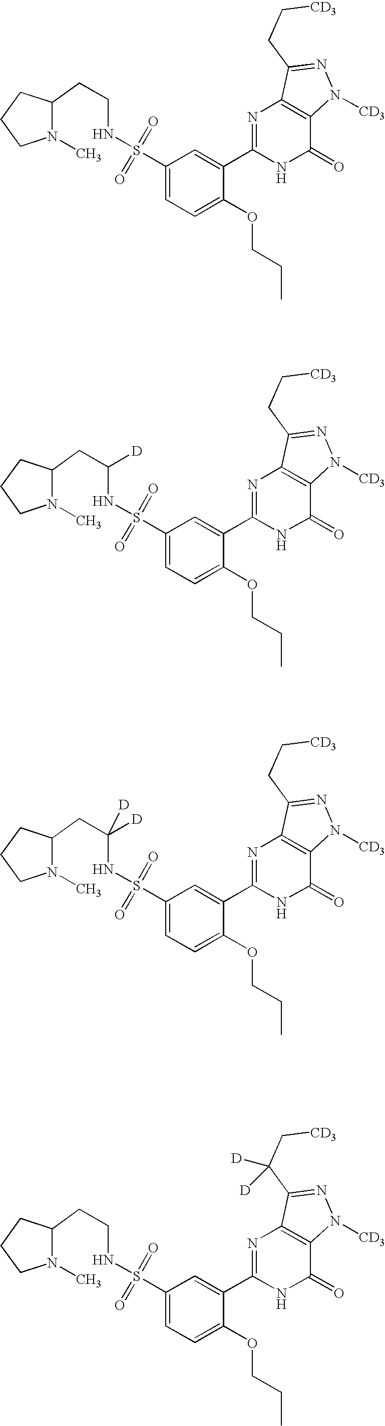

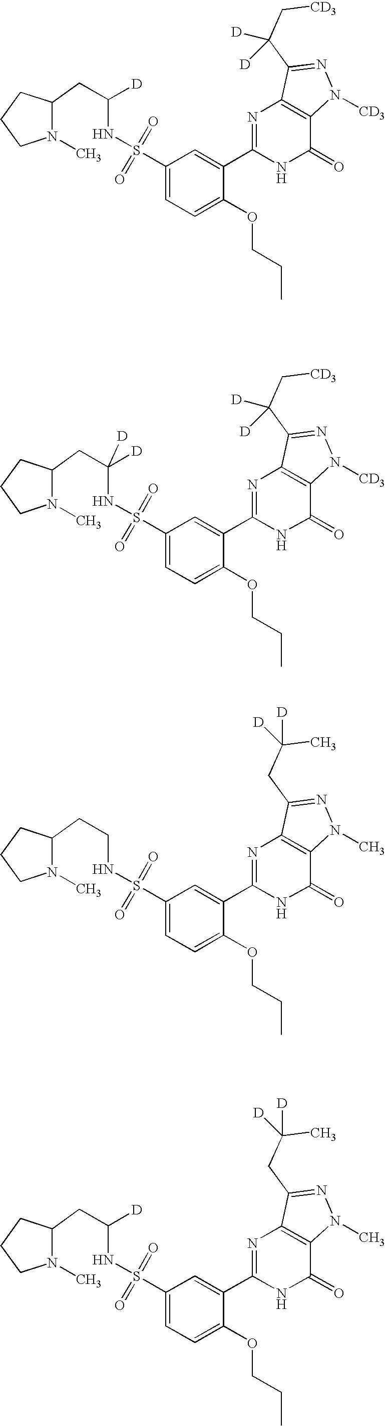

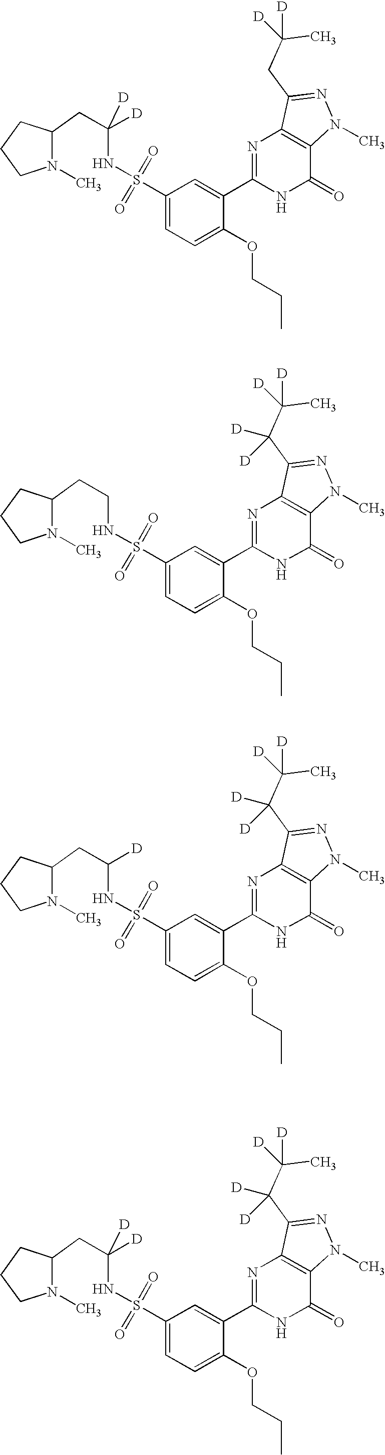

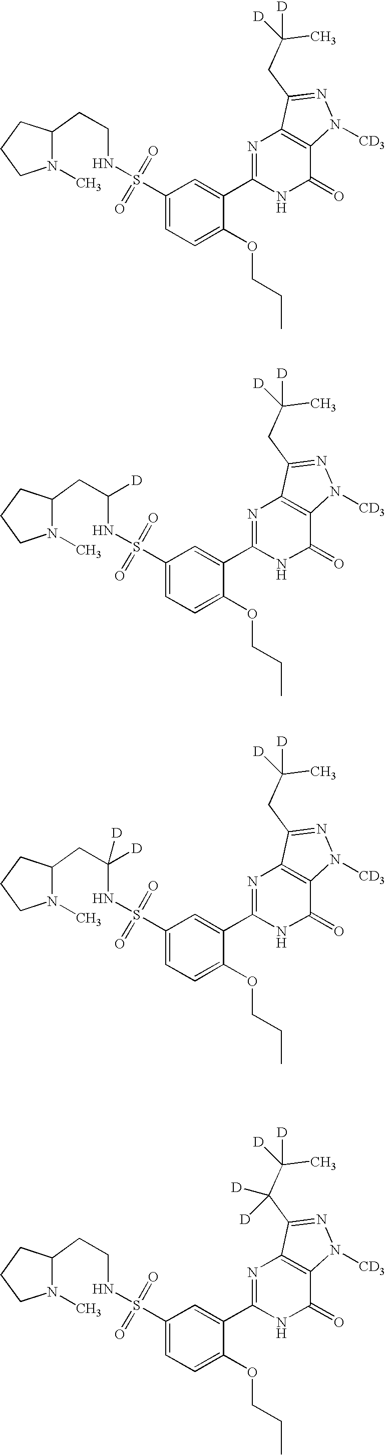

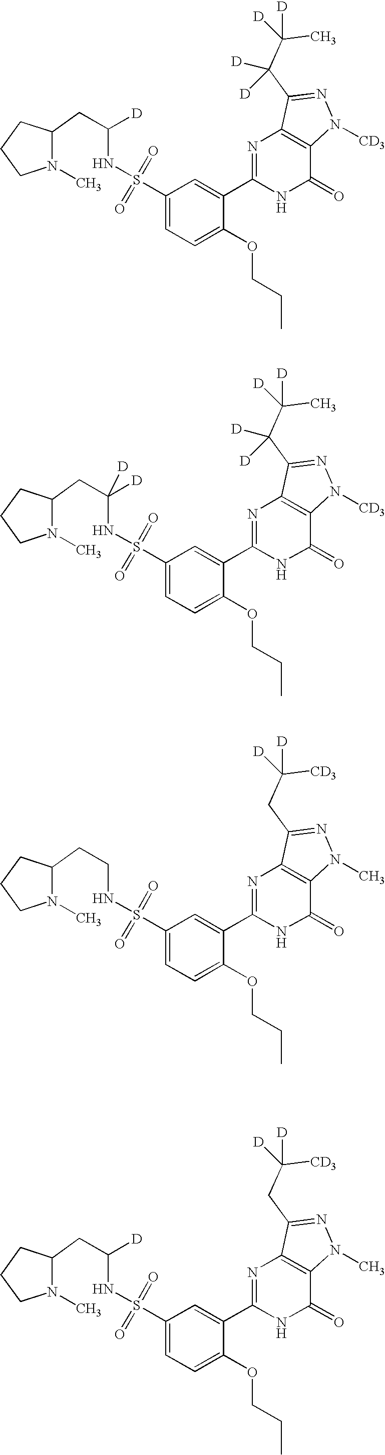

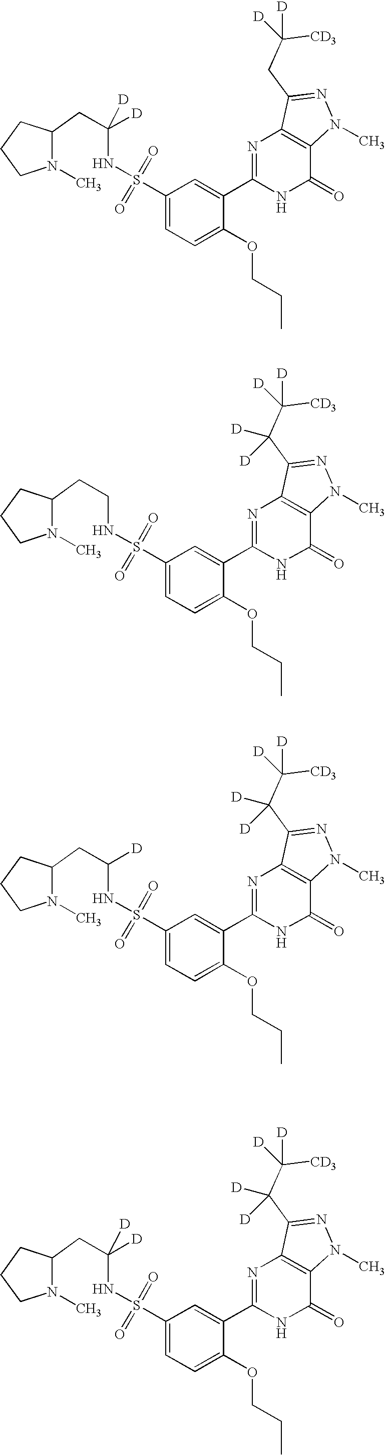

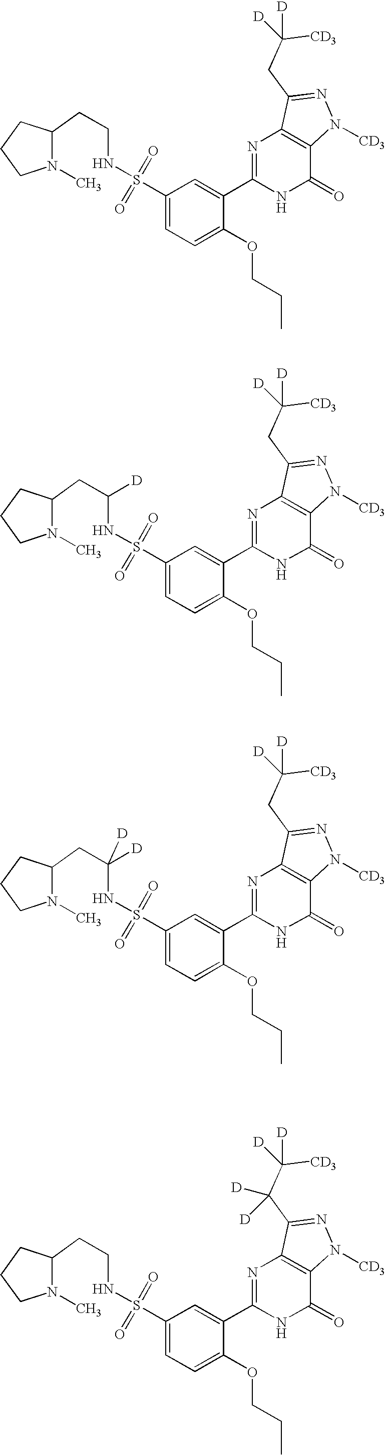

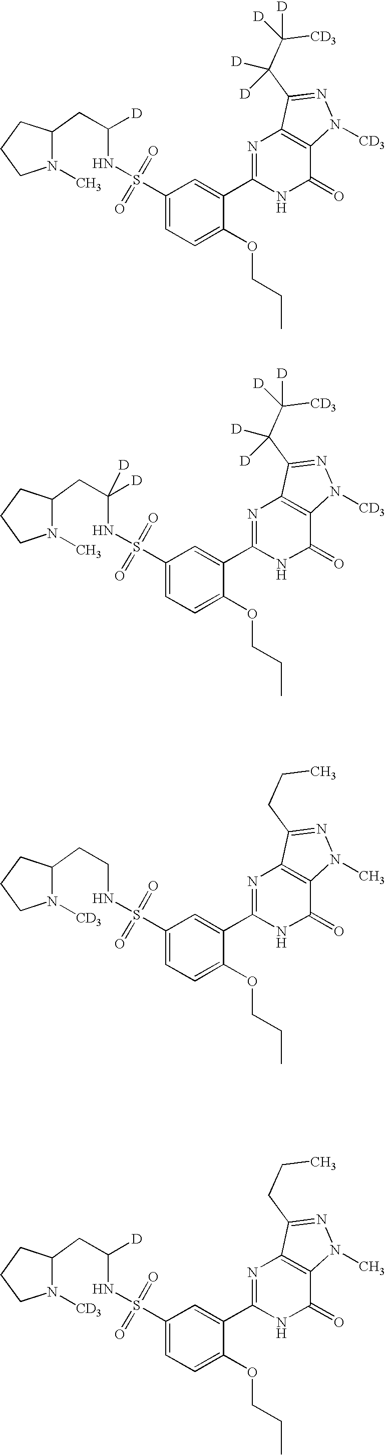

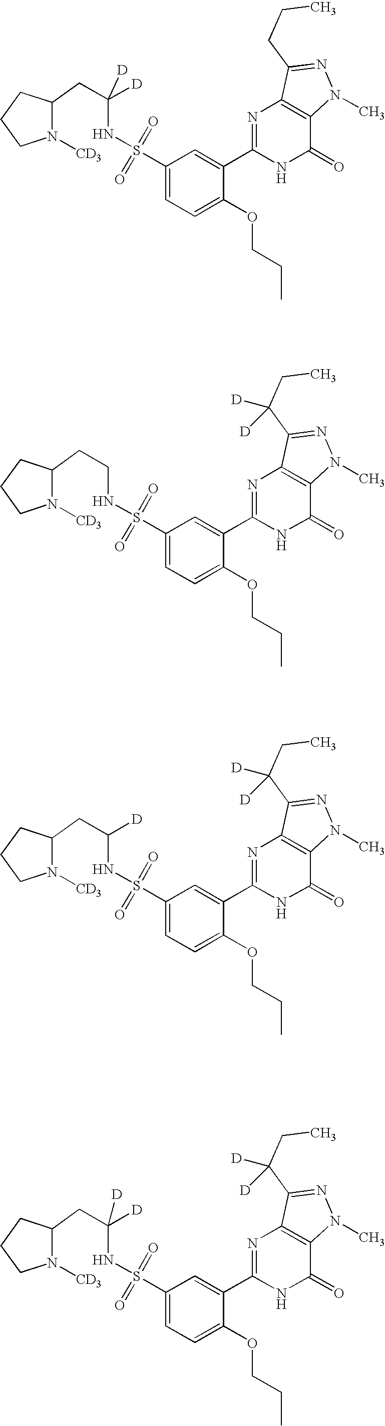

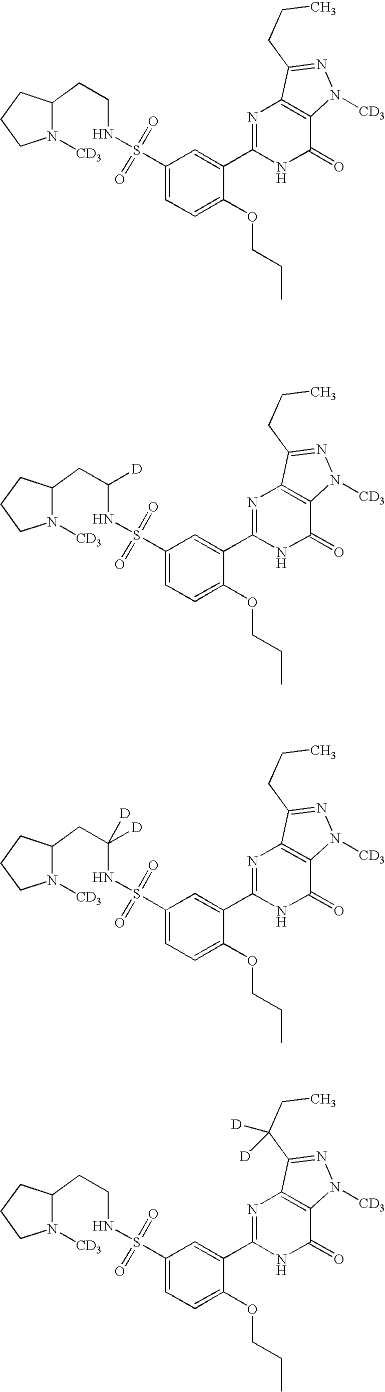

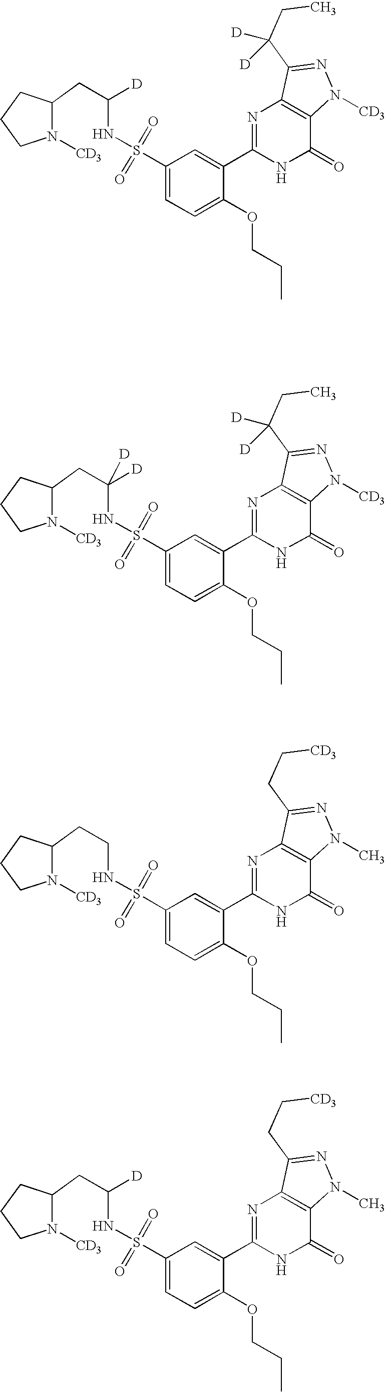

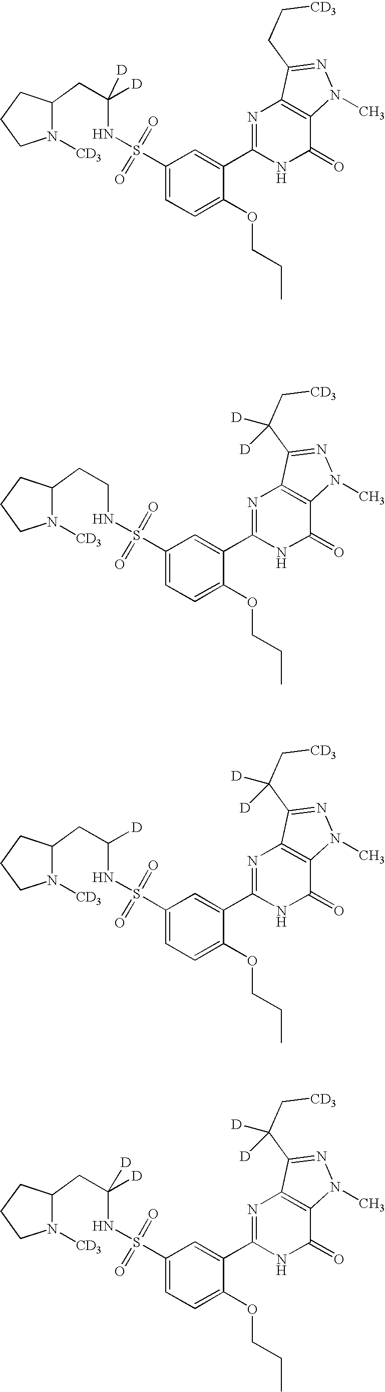

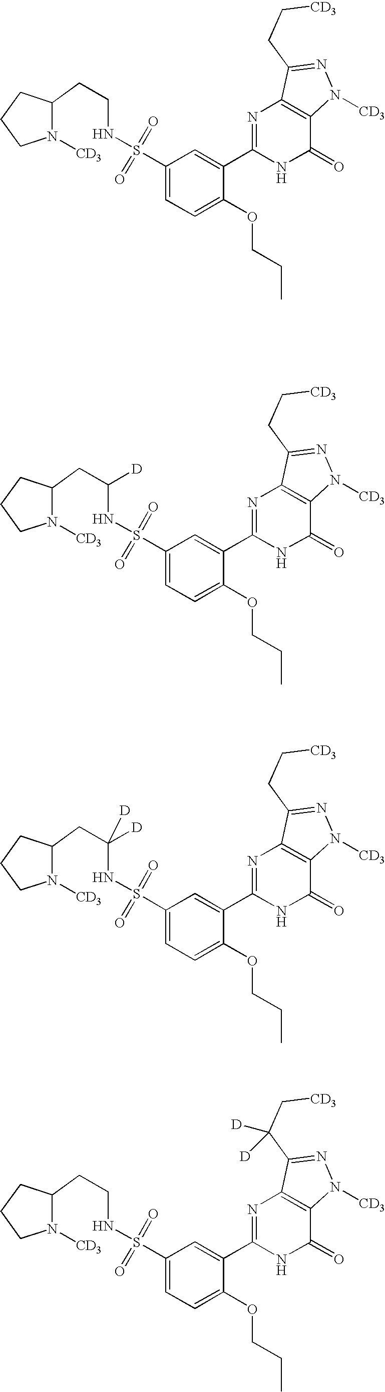

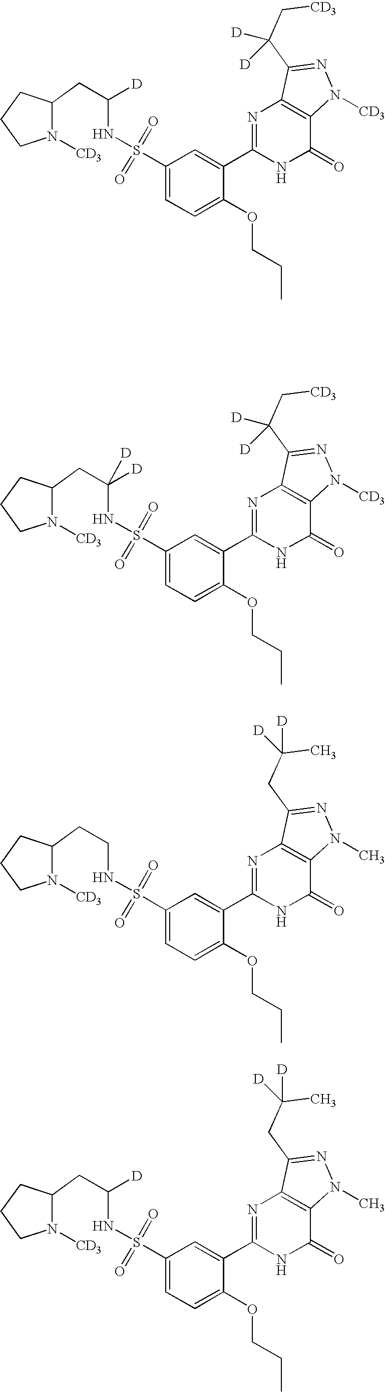

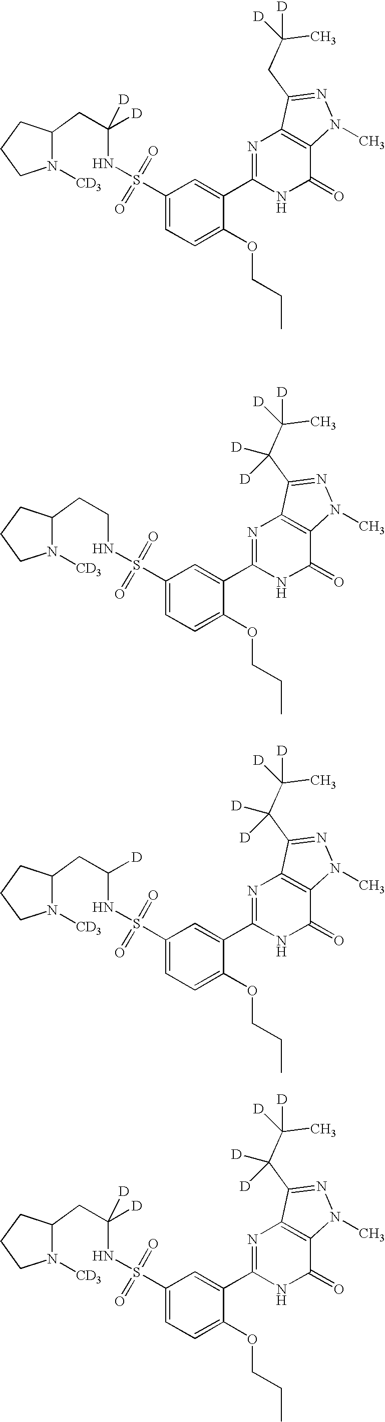

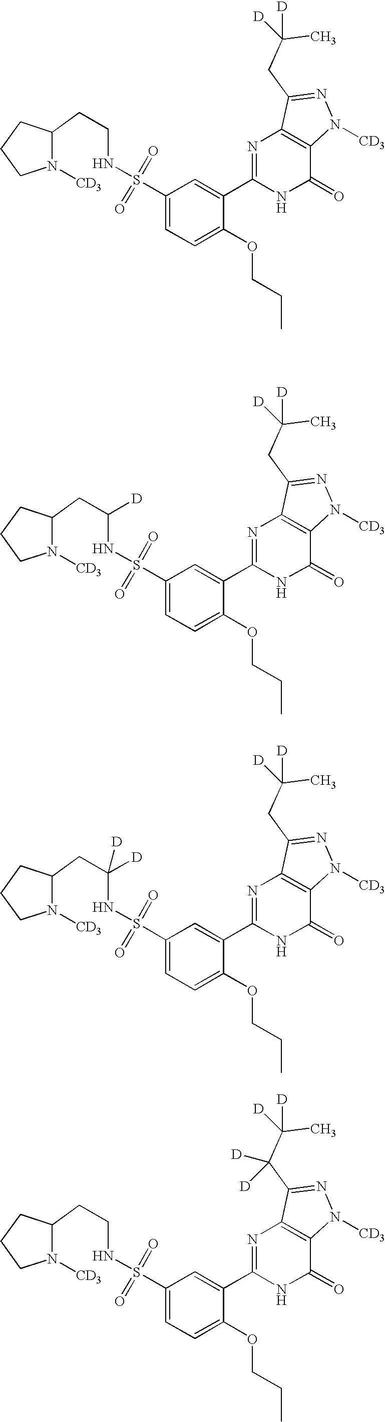

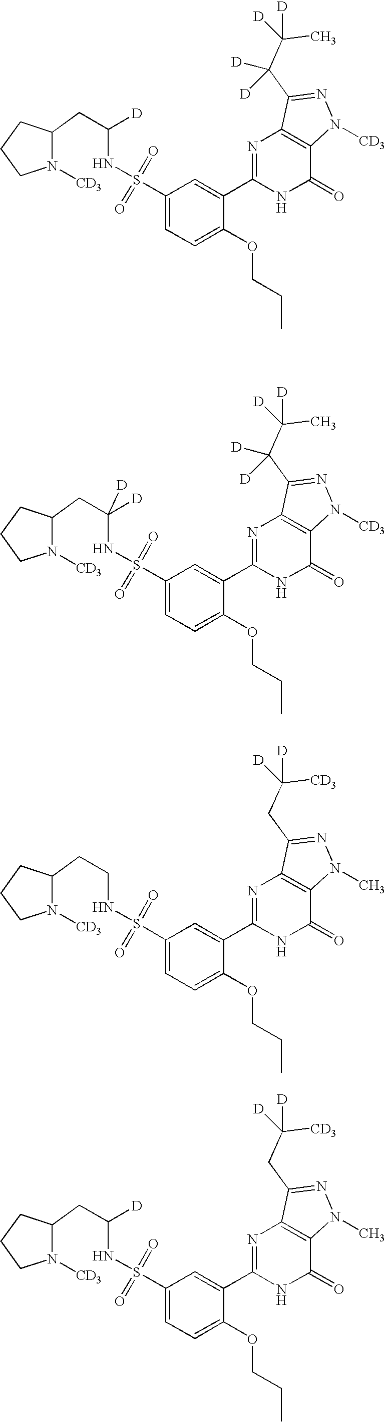

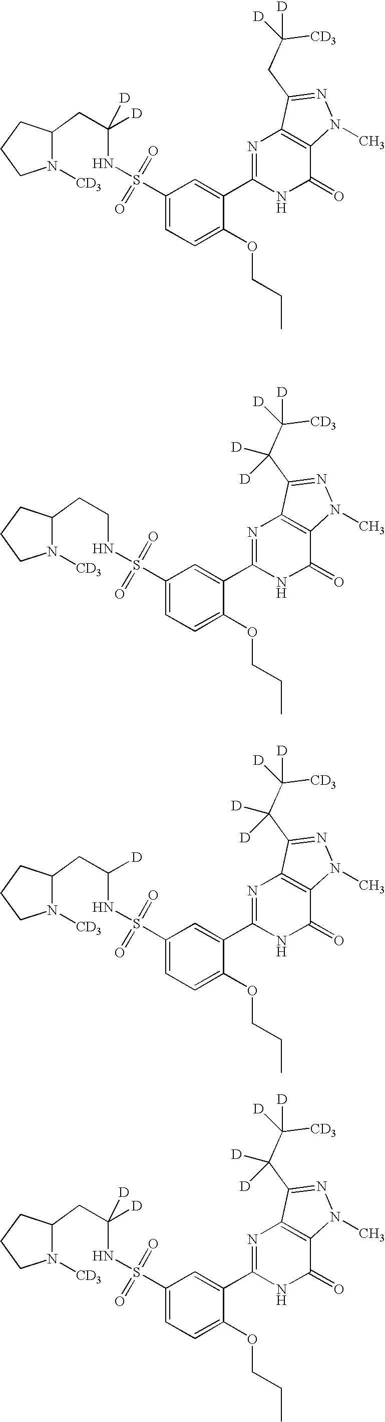

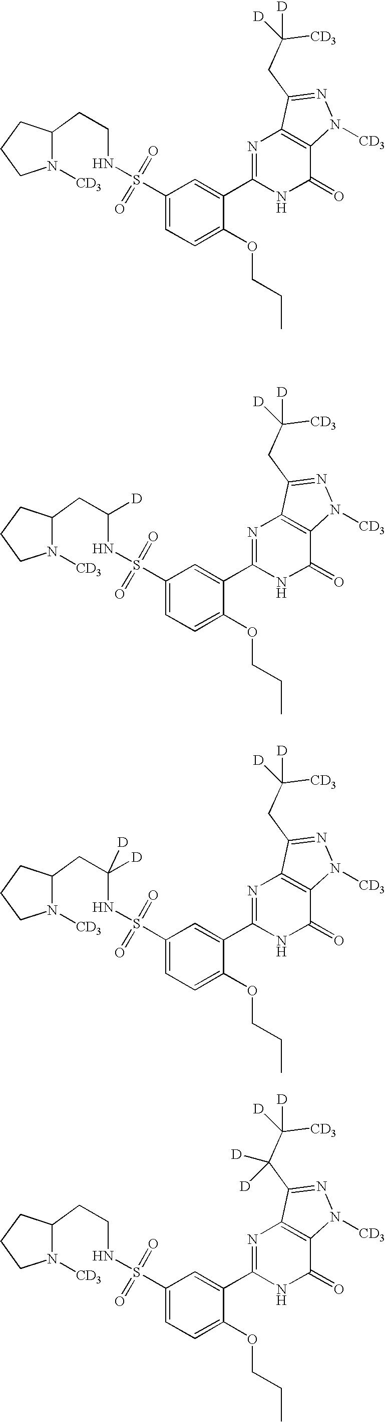

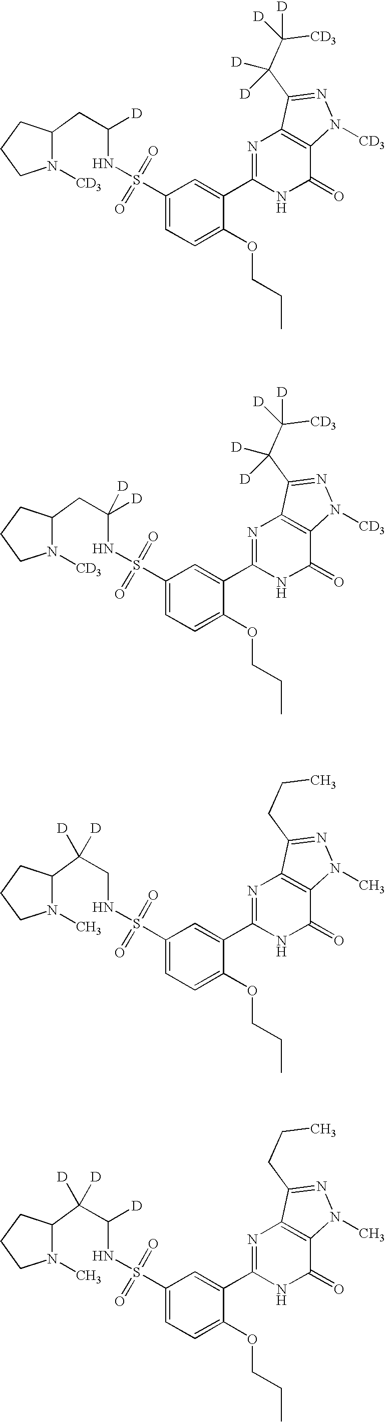

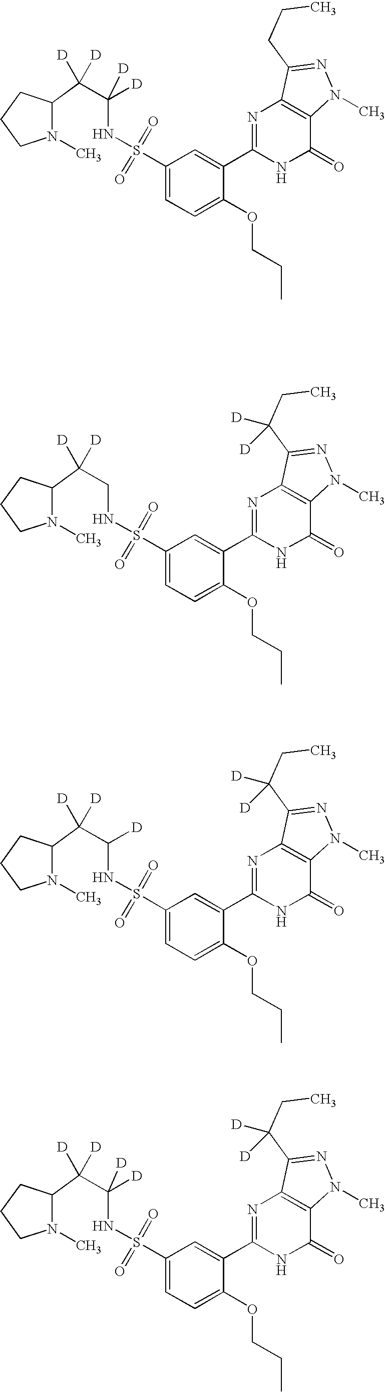

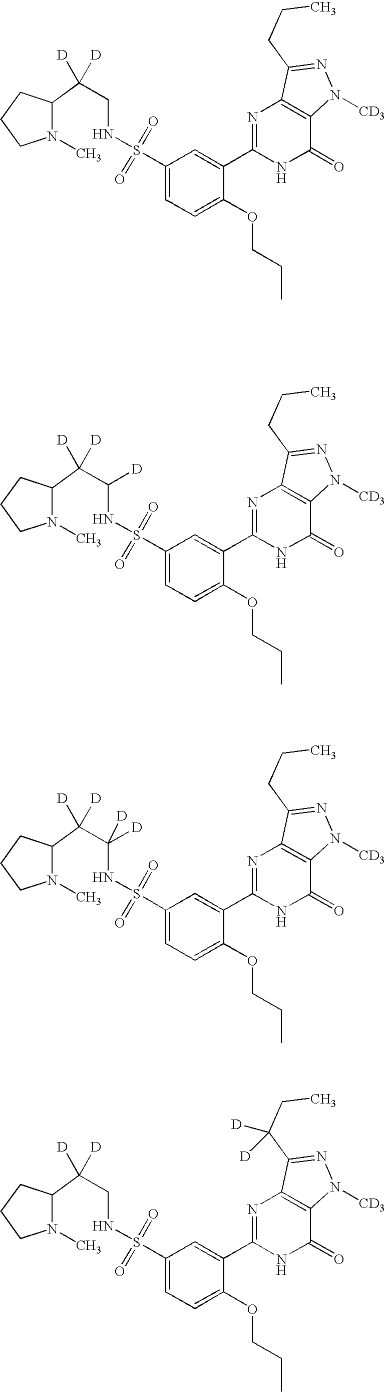

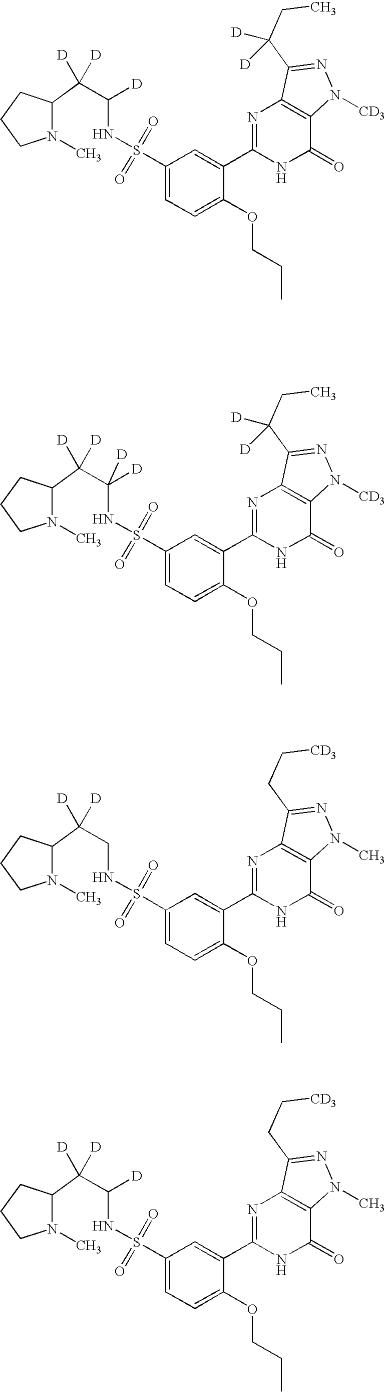

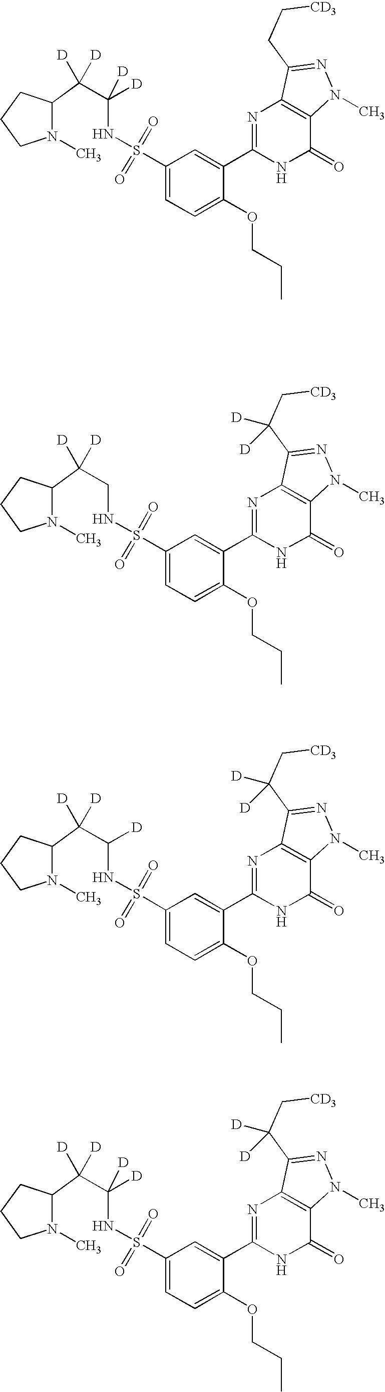

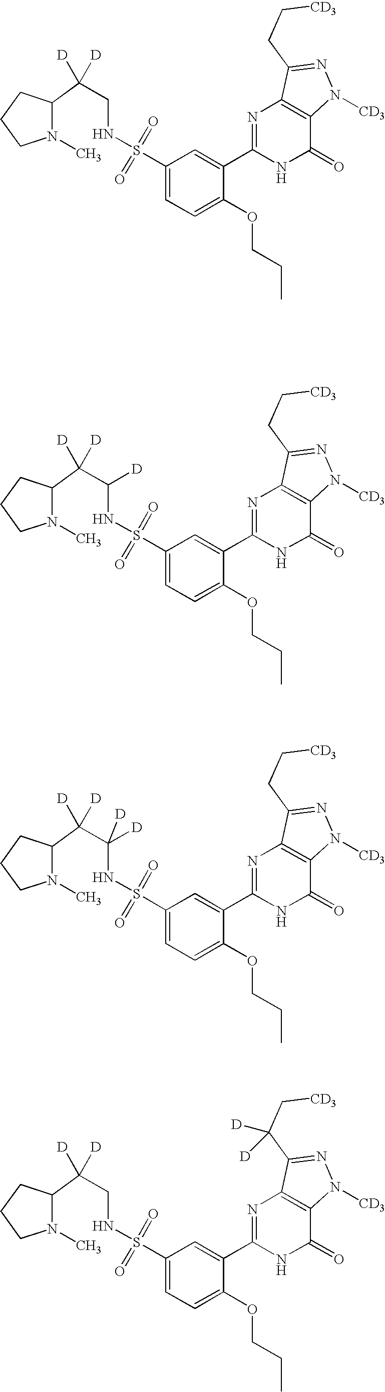

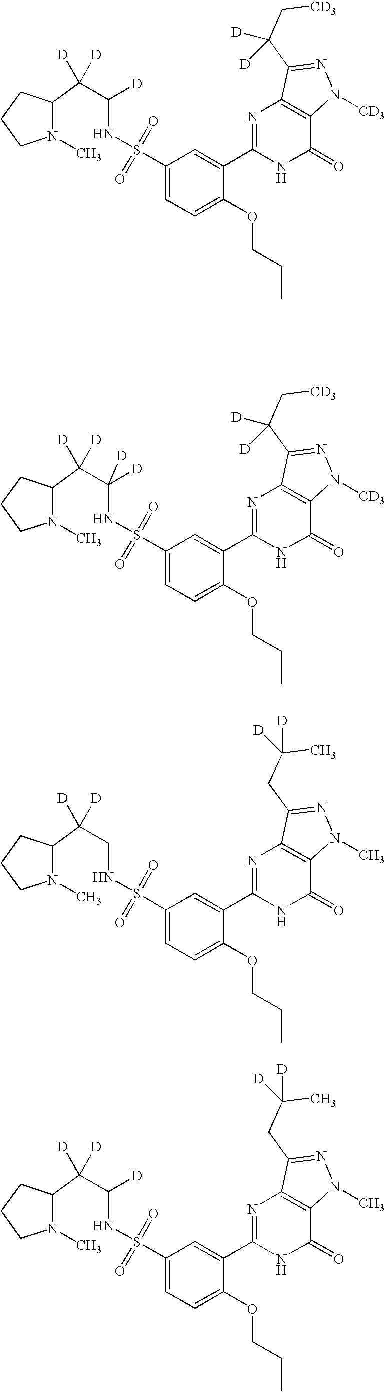

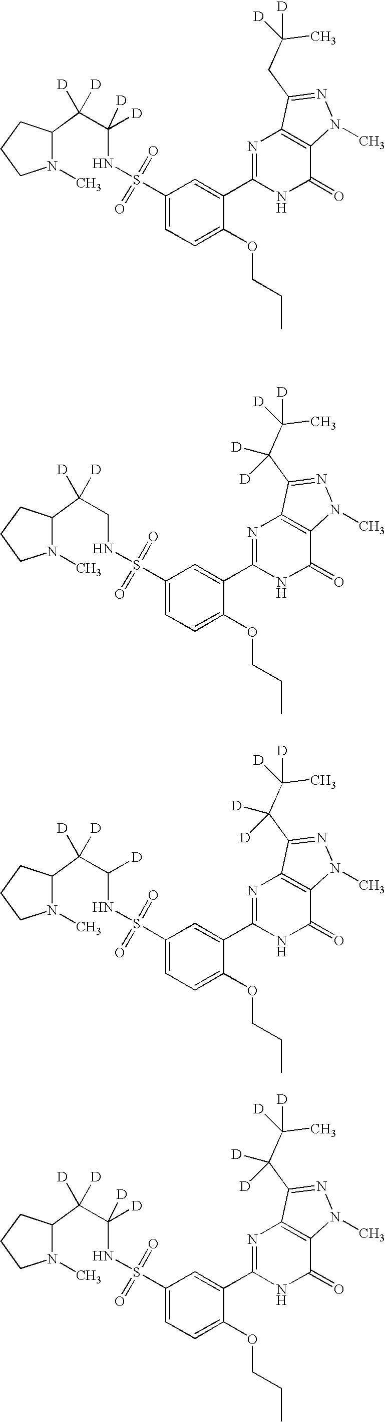

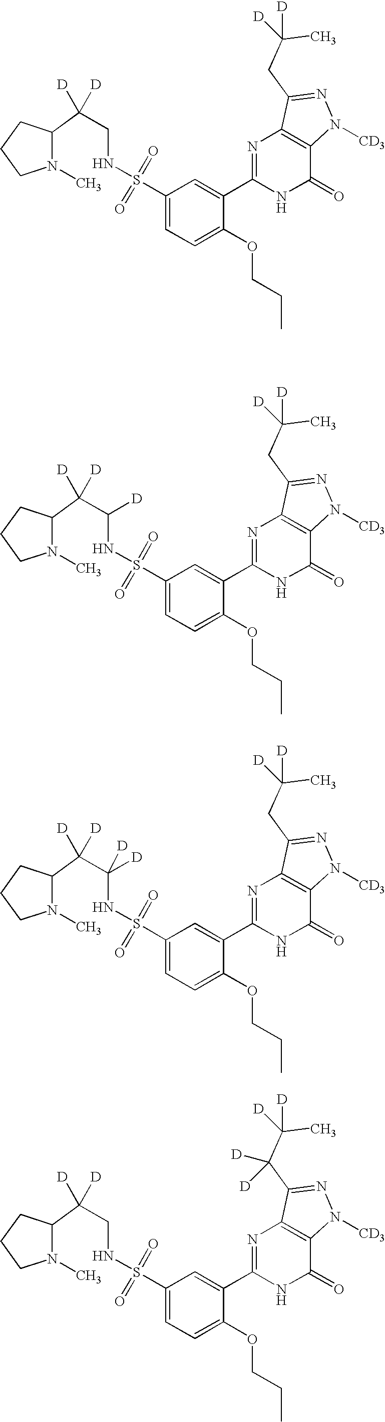

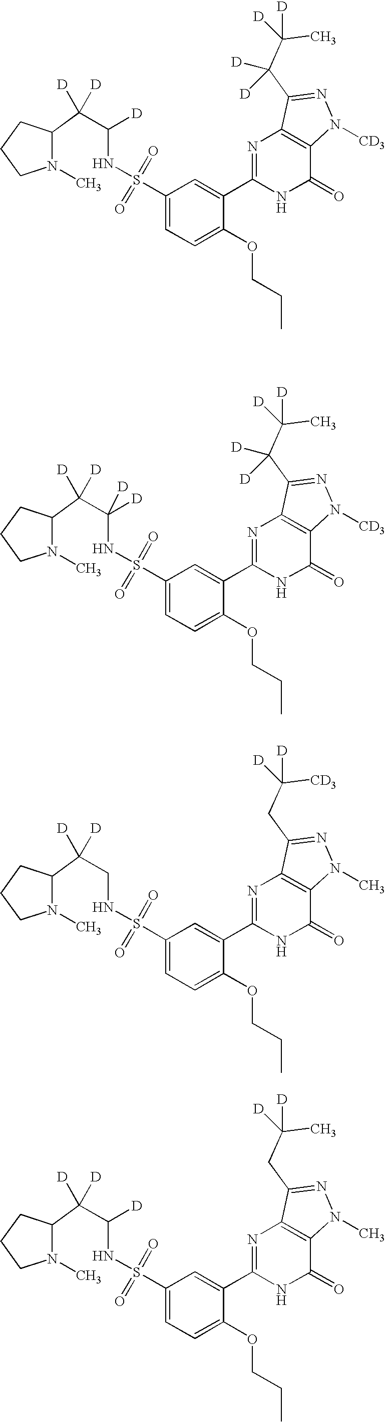

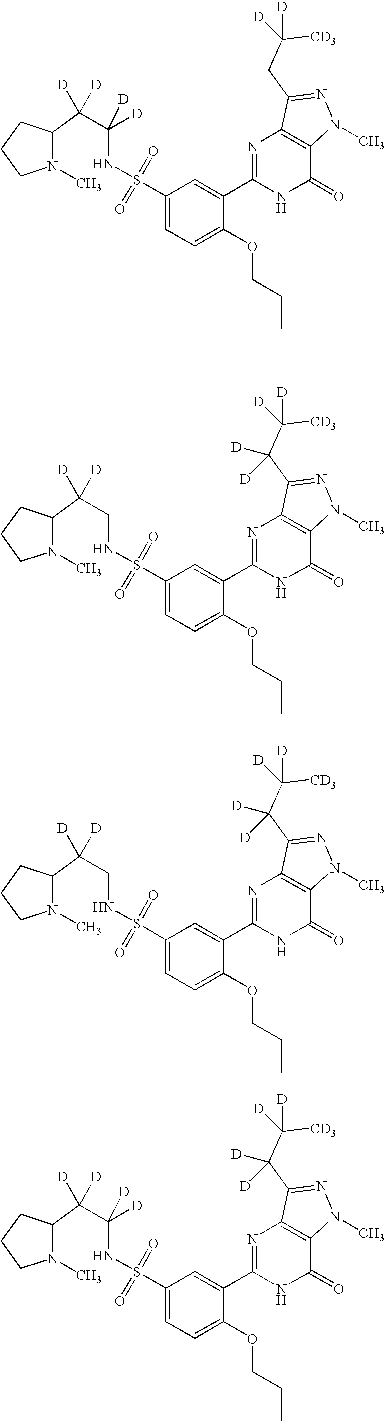

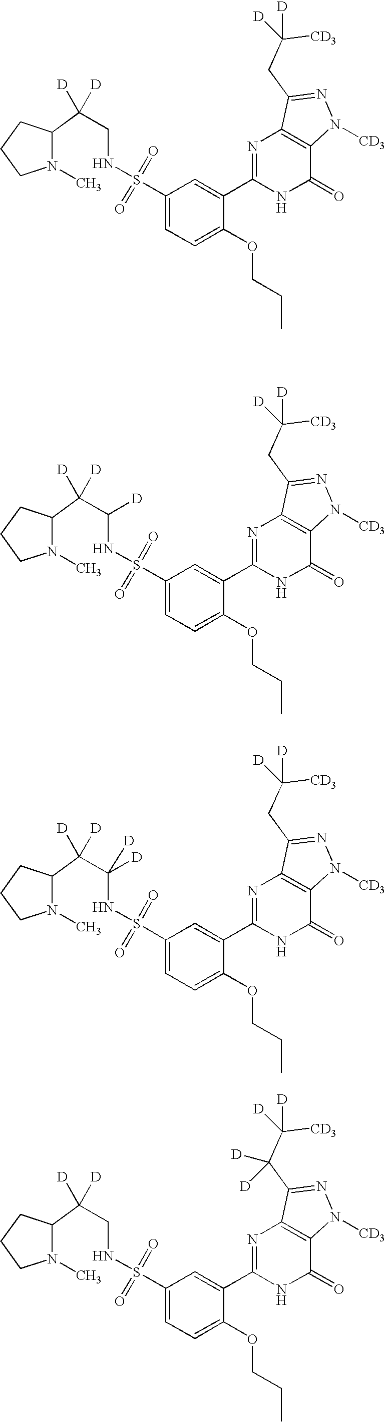

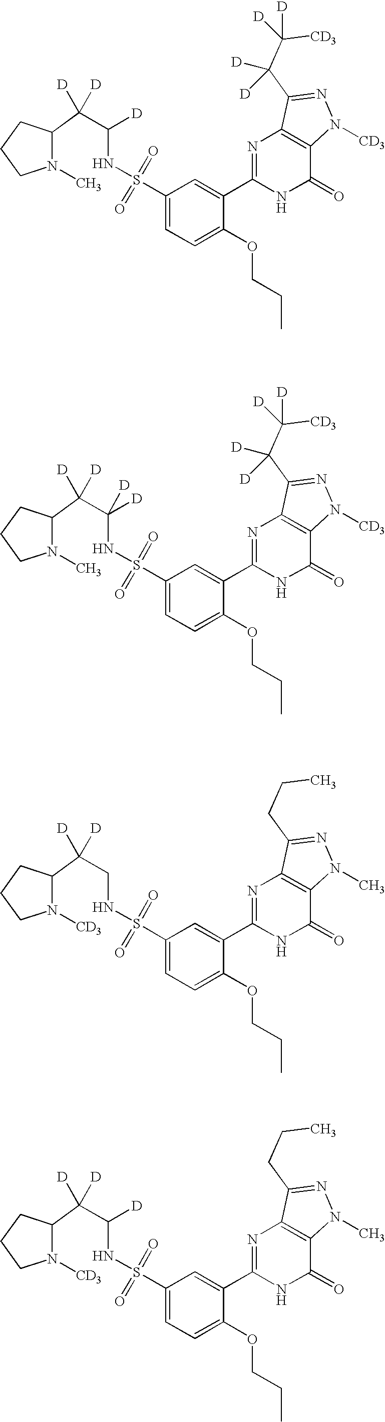

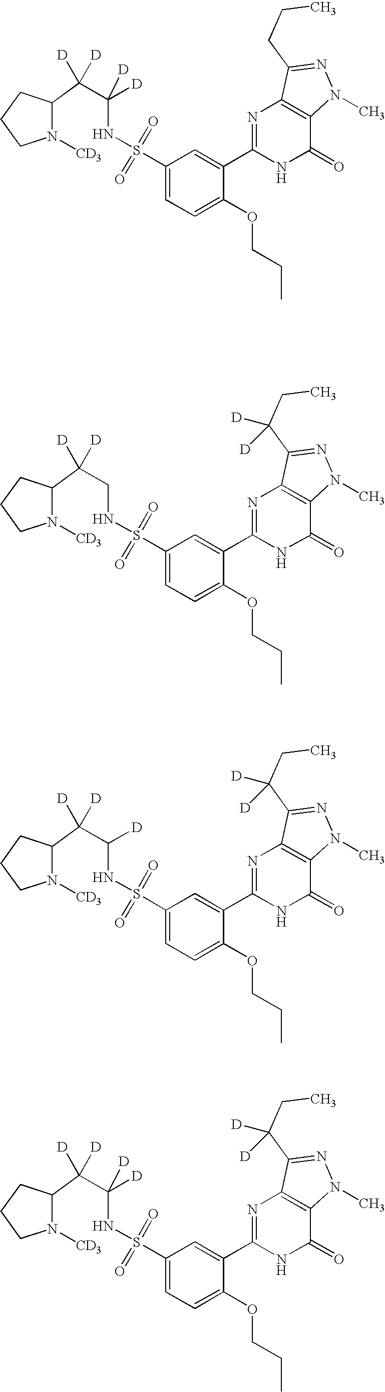

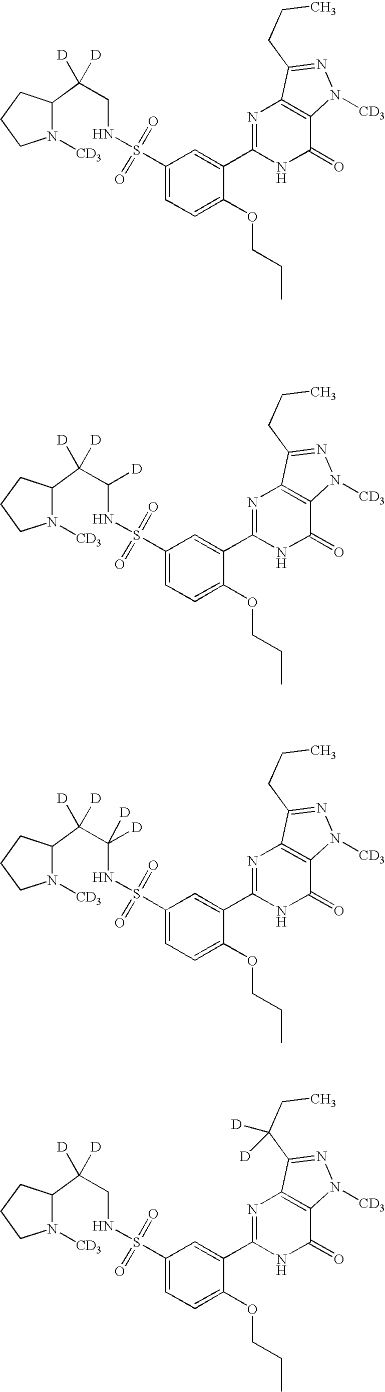

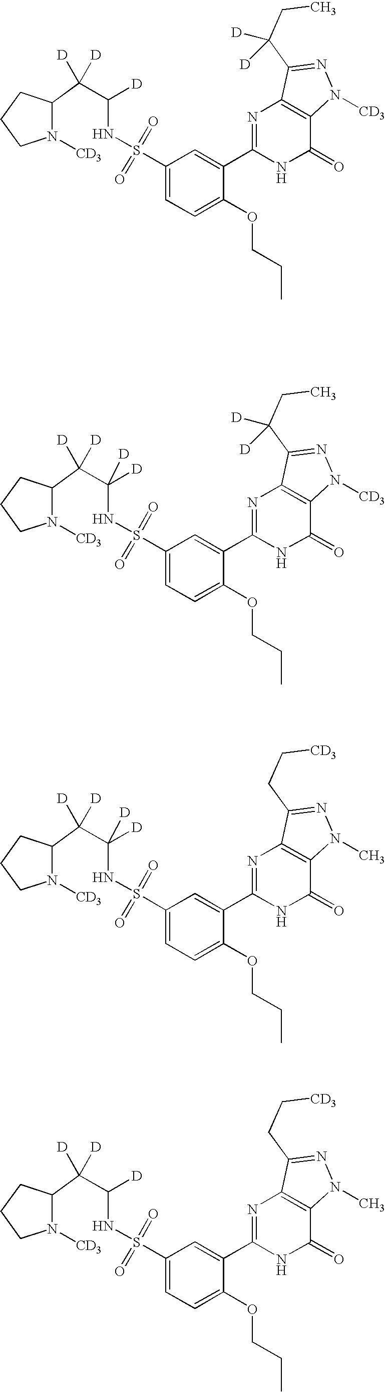

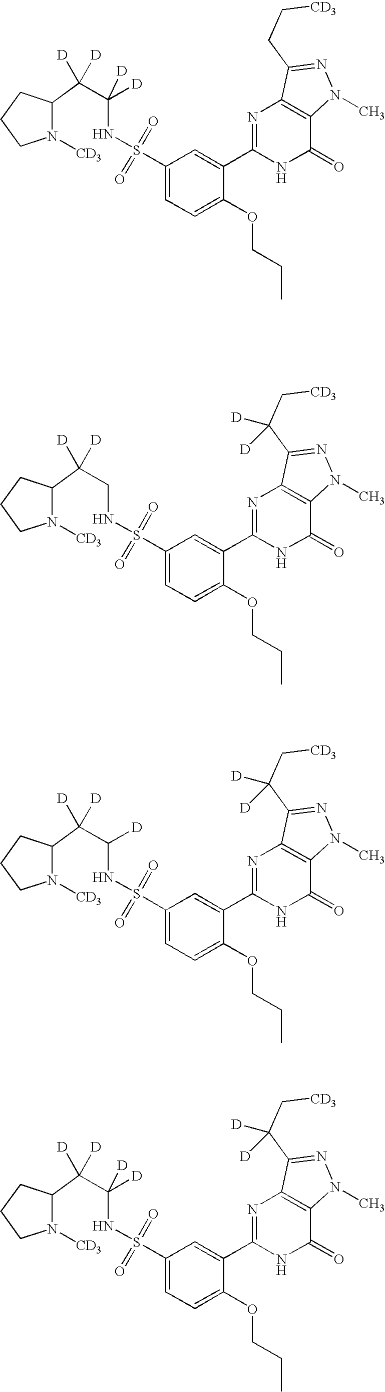

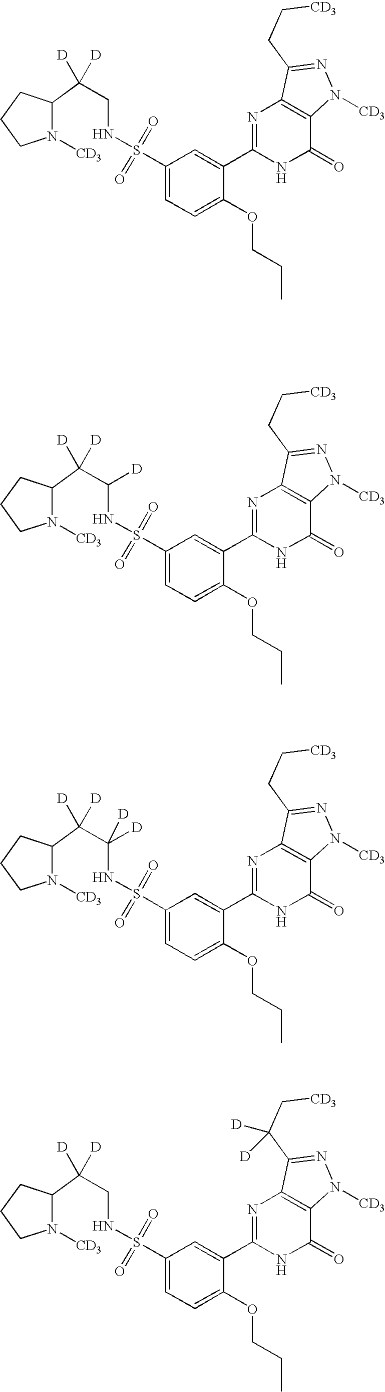

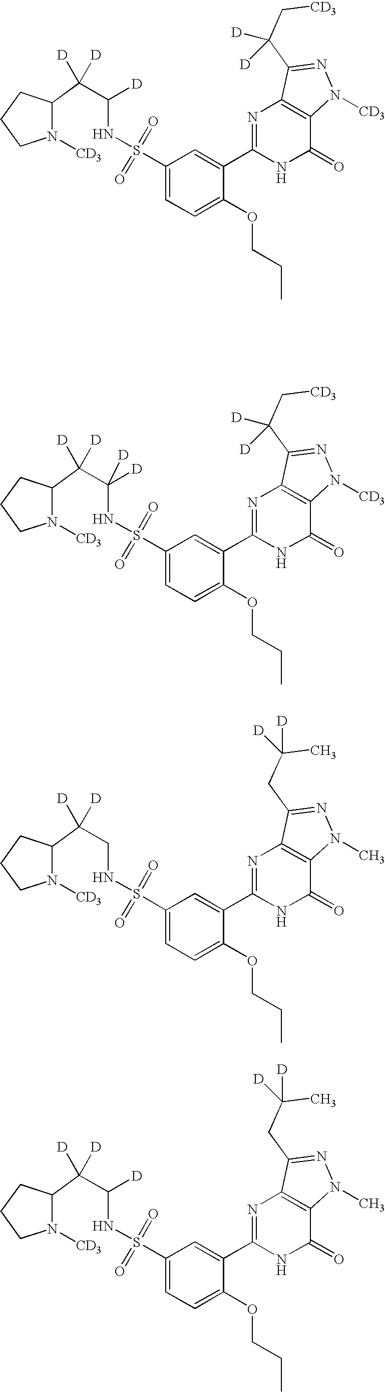

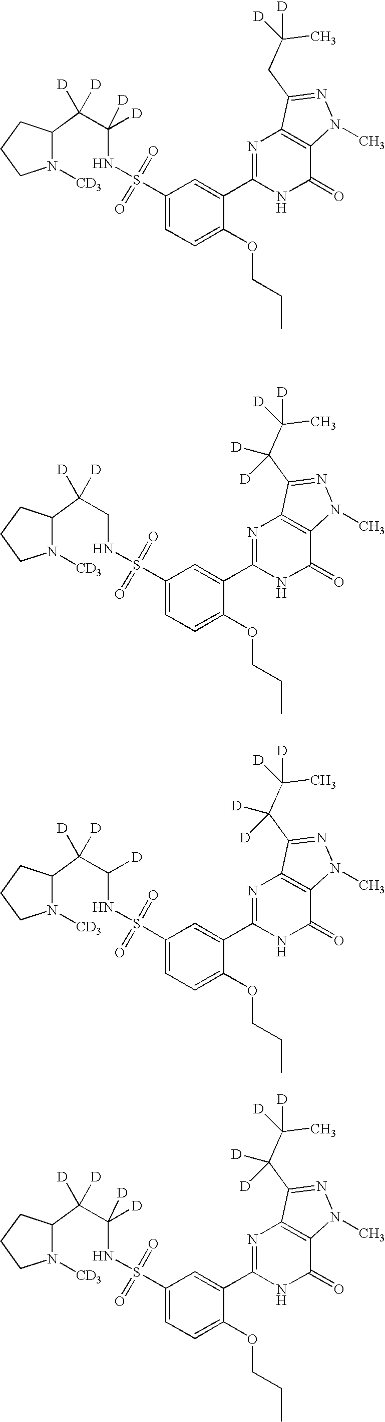

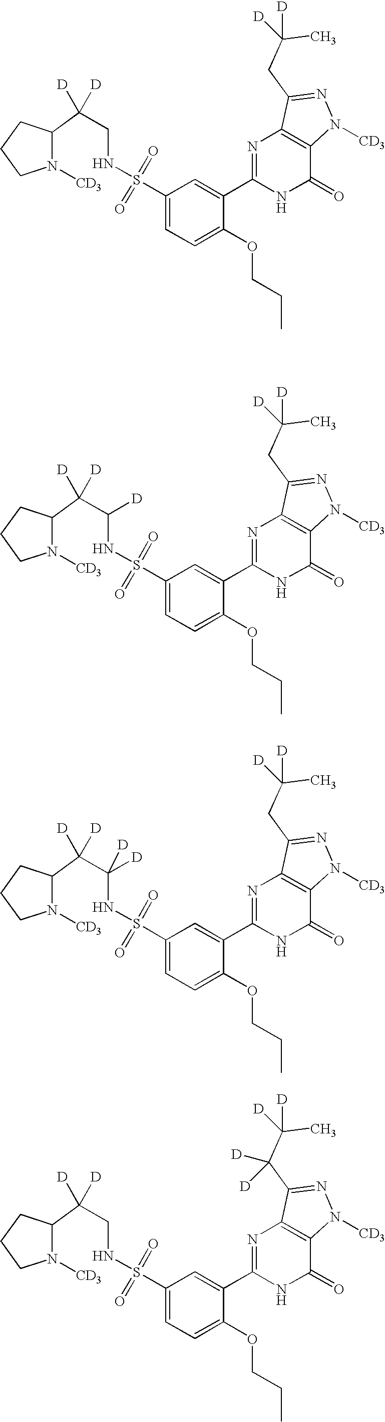

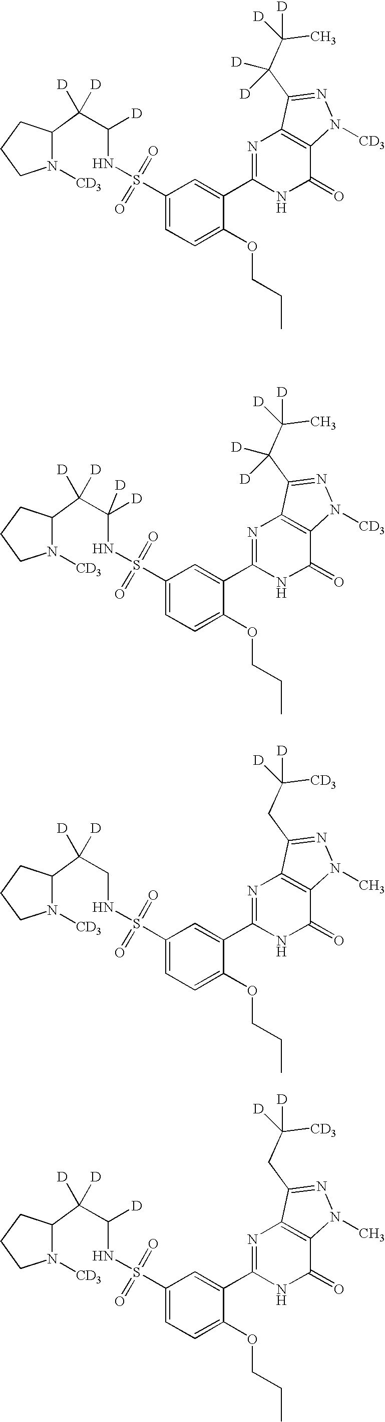

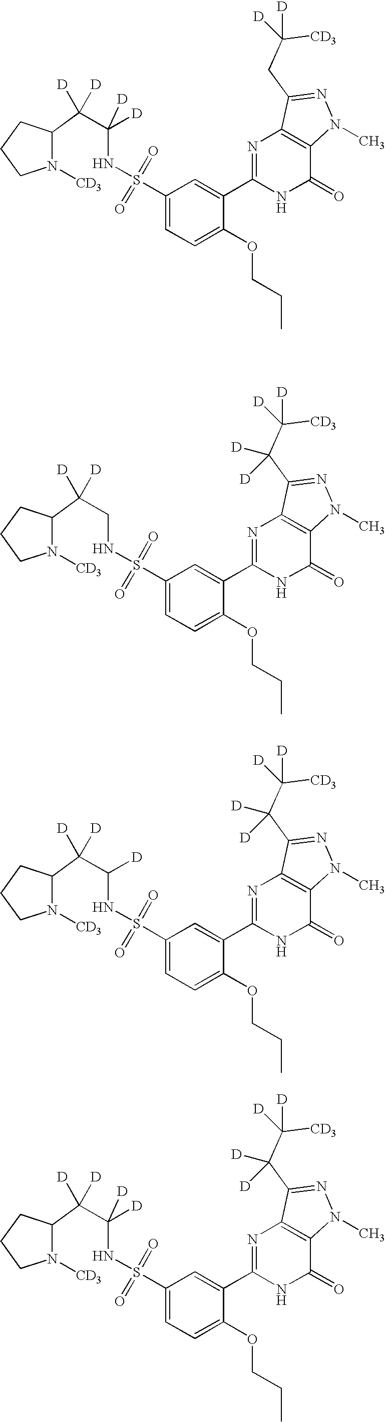

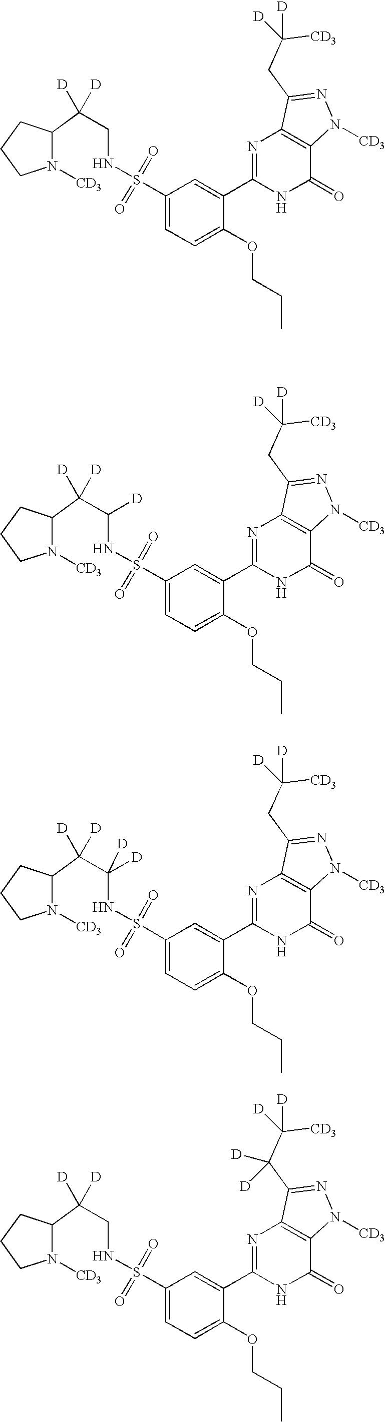

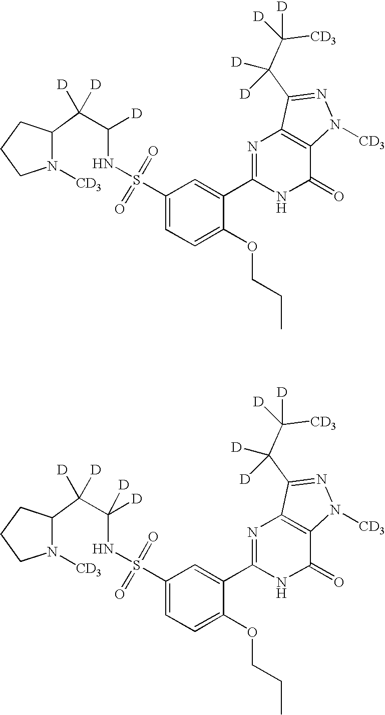

or a pharmaceutically acceptable salt, solvate, or prodrug thereof.

In another embodiment, at least one of the indicated D's independently has deuterium enrichment of no less than about 1%, no less than about 5%, no less than about 10%, no less than about 20%, no less than about 50%, no less than about 70%, no less than about 80%, no less than about 90%, or no less than about 98%.

In a further embodiment, said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

In other embodiments, R₁ is hydrogen. In yet other embodiments, R₂ is hydrogen. In still other embodiments, R₃ is hydrogen. In yet other embodiments, R₄ is hydrogen. In still other embodiments, R₅ is hydrogen. In yet other embodiments, R₆ is hydrogen. In still other embodiments, R₇ is hydrogen. In still other embodiments, R₈ is hydrogen. In some embodiments, R₉ is hydrogen. In other embodiments, R₁₀ is hydrogen. In yet other embodiments, R₁₁ is hydrogen. In still other embodiments, R₁₂ is hydrogen. In yet other embodiments, R₁₃ is hydrogen. In other embodiments, R₁₄ is hydrogen. In certain embodiments, R₁₅ is hydrogen. In other embodiments, R₁₆ is hydrogen. In other embodiments, R₁₇ is hydrogen. In yet other embodiments, R₁₈ is hydrogen. In still other embodiments, R₁₉ is hydrogen. In yet other embodiments, R₂₀ is hydrogen. In other embodiments, R₂₁ is hydrogen. In still other embodiments, R₂₂ is hydrogen. In other embodiments, R₂₃ is hydrogen. In yet other embodiments, R₂₄ is hydrogen. In certain embodiments, R₂₅ is hydrogen. In other embodiments, R₂₆ is hydrogen. In yet other embodiments, R₂₇ is hydrogen. In certain embodiments, R₂₈ is hydrogen. In yet other embodiments, R₂₉ is hydrogen. In certain embodiments, R₃₀ is hydrogen. In yet other embodiments, R₃₁ is hydrogen. In certain embodiments, R₃₂ is hydrogen. In other embodiments, R₃₃ is hydrogen. In yet other embodiments, R₃₄ is hydrogen. In certain embodiments, R₃₅ is hydrogen. In yet other embodiments, R₃₆ is hydrogen.

In other embodiments, R₁ is deuterium. In yet other embodiments, R₂ is deuterium. In still other embodiments, R₃ is deuterium. In yet other embodiments, R₄ is deuterium. In still other embodiments, R₅ is deuterium. In yet other embodiments, R₆ is deuterium. In still other embodiments, R₇ is deuterium. In still other embodiments, R₈ is deuterium. In some embodiments, R₉ is deuterium. In other embodiments, R₁₀ is deuterium. In yet other embodiments, R₁₁ is deuterium. In still other embodiments, R₁₂ is deuterium. In yet other embodiments, R₁₃ is deuterium. In other embodiments, R₁₄ is deuterium. In certain embodiments, R₁₅ is deuterium. In other embodiments, R₁₆ is deuterium. In yet other embodiments, R₁₇ is deuterium. In some embodiments, R₁₈ is deuterium. In other embodiments, R₁₉ is deuterium. In yet other embodiments, R₂₀ is deuterium. In still other embodiments, R₂₁ is deuterium. In other embodiments, R₂₂ is deuterium. In other embodiments, R₂₃ is deuterium. In certain embodiments, R₂₄ is deuterium. In certain embodiments, R₂₅ is deuterium. In other embodiments, R₂₆ is deuterium. In yet other embodiments, R₂₇ is deuterium. In some embodiments, R₂₈ is deuterium. In yet other embodiments, R₂₉ is deuterium. In some embodiments, R₃₀ is deuterium. In certain embodiments, R₃₁ is deuterium. In other embodiments, R₃₂ is deuterium. In yet other embodiments, R₃₃ is deuterium. In some embodiments, R₃₄ is deuterium. In yet other embodiments, R₃₅ is deuterium. In some embodiments, R₃₆ is deuterium.

In certain embodiments, the compound as disclosed herein contains about 60% or more by weight of the (−)-enantiomer of the compound and about 40% or less by weight of (+)-enantiomer of the compound. In certain embodiments, the compound as disclosed herein contains about 70% or more by weight of the (−)-enantiomer of the compound and about 30% or less by weight of (+)-enantiomer of the compound. In certain embodiments, the compound as disclosed herein contains about 80% or more by weight of the (−)-enantiomer of the compound and about 20% or less by weight of (+)-enantiomer of the compound. In certain embodiments, the compound as disclosed herein contains about 90% or more by weight of the (−)-enantiomer of the compound and about 10% or less by weight of the (+)-enantiomer of the compound. In certain embodiments, the compound as disclosed herein contains about 95% or more by weight of the (−)-enantiomer of the compound and about 5% or less by weight of (+)-enantiomer of the compound. In certain embodiments, the compound as disclosed herein contains about 99% or more by weight of the (−)-enantiomer of the compound and about 1% or less by weight of (+)-enantiomer of the compound.

In certain embodiments, the compound as disclosed herein contains about 60% or more by weight of the (+)-enantiomer of the compound and about 40% or less by weight of (−)-enantiomer of the compound. In certain embodiments, the compound as disclosed herein contains about 70% or more by weight of the (+)-enantiomer of the compound and about 30% or less by weight of (−)-enantiomer of the compound. In certain embodiments, the compound as disclosed herein contains about 80% or more by weight of the (+)-enantiomer of the compound and about 20% or less by weight of (−)-enantiomer of the compound. In certain embodiments, the compound as disclosed herein contains about 90% or more by weight of the (+)-enantiomer of the compound and about 10% or less by weight of the (−)-enantiomer of the compound. In certain embodiments, the compound as disclosed herein contains about 95% or more by weight of the (+)-enantiomer of the compound and about 5% or less by weight of (−)-enantiomer of the compound. In certain embodiments, the compound as disclosed herein contains about 99% or more by weight of the (+)-enantiomer of the compound and about 1% or less by weight of (−)-enantiomer of the compound.

The deuterated compound as disclosed herein may also contain less prevalent isotopes of other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, without being bound by any theory, the compound disclosed herein may expose a patient to a maximum of about 0.000005% D₂O or about 0.00001% DHO, assuming that all of the C-D bonds in the compound as disclosed herein are metabolized and released as D₂O or DHO. This quantity is a small fraction of the naturally occurring background levels of D₂O or DHO in circulation. In certain embodiments, the levels of D₂O shown to cause toxicity in animals is far greater than the maximally achieved exposure dose of the deuterium enriched compounds disclosed herein. Thus, in certain embodiments, the deuterium-enriched compound disclosed herein should not cause any additional toxicity because of the use of deuterium.

In one embodiment, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in van Herk et al Journal of Medicinal Chemistry, 2003, 46(18), 3945-3951, Dale et al Organic Process Research & Development 2000, 4(1), 17-22, EP 463,756, and U.S. Pat. No. 6,844,436, and references cited therein and routine modifications thereof. Compounds as disclosed herein can also be prepared as shown in any of the following schemes and routine modifications thereof.

Deuterium can also be incorporated to various positions having an exchangeable proton, such as the amide N—H, via proton-deuterium equilibrium exchange. For example, to introduce deuterium at R₁₀, this proton may be replaced with deuterium selectively or non-selectively through a proton-deuterium exchange method known in the art.

It is to be understood that the compounds disclosed herein may contain one or more chiral centers, chiral axes, and/or chiral planes, as described in “Stereochemistry of Carbon Compounds” Eliel and Wilen, John Wiley & Sons, New York, 1994, pp. 1119-1190. Such chiral centers, chiral axes, and chiral planes may be of either the (R) or (S) configuration, or may be a mixture thereof.

Another method for characterizing a composition containing a compound having at least one chiral center is by the effect of the composition on a beam of polarized light. When a beam of plane polarized light is passed through a solution of a chiral compound, the plane of polarization of the light that emerges is rotated relative to the original plane. This phenomenon is known as optical activity, and compounds that rotate the plane of polarized light are said to be optically active. One enantiomer of a compound will rotate the beam of polarized light in one direction, and the other enantiomer will rotate the beam of light in the opposite direction. The enantiomer that rotates the polarized light in the clockwise direction is the (+) enantiomer and the enantiomer that rotates the polarized light in the counterclockwise direction is the (−) enantiomer. Included within the scope of the compositions described herein are compositions containing between 0 and 100% of the (+) and/or (−) enantiomer of compounds as disclosed herein.

Where a compound as disclosed herein contains an alkenyl or alkenylene group, the compound may exist as one or mixture of geometric cis/trans (or Z/E) isomers. Where structural isomers are interconvertible via a low energy barrier, the compound as disclosed herein may exist as a single tautomer or a mixture of tautomers. This can take the form of proton tautomerism in the compound as disclosed herein that contains for example, an imino, keto, or oxime group; or so-called valence tautomerism in the compound that contain an aromatic moiety. It follows that a single compound may exhibit more than one type of isomerism.

The compounds disclosed herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the compound as disclosed herein contains an acidic or basic moiety, the compound may also be embodied as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The compound as disclosed herein may also be designed as a prodrug, which is a functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asghamejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

### Pharmaceutical Composition

Disclosed herein are pharmaceutical compositions comprising a compound as disclosed herein as an active ingredient, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, in a pharmaceutically acceptable vehicle, carrier, diluent, or excipient, or a mixture thereof, in combination with one or more pharmaceutically acceptable excipients or carriers.

Disclosed herein are pharmaceutical compositions in modified release dosage forms, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more release controlling excipients or carriers as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients or carriers.

Further disclosed herein are pharmaceutical compositions in enteric coated dosage forms, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more release controlling excipients or carriers for use in an enteric coated dosage form. The pharmaceutical compositions may also comprise non-release controlling excipients or carriers.

Additionally disclosed are pharmaceutical compositions in a dosage form that has an instant releasing component and at least one delayed releasing component, and is capable of giving a discontinuous release of the compound in the form of at least two consecutive pulses separated in time from 0.1 up to 24 hours. The pharmaceutical compositions comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more release controlling and non-release controlling excipients or carriers, such as those excipients or carriers suitable for a disruptable semi-permeable membrane and as swellable substances.

Disclosed herein also are pharmaceutical compositions in a dosage form for oral administration to a subject, which comprise a compound as disclosed herein, including a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Disclosed herein are pharmaceutical compositions that comprise about 0.1 to about 1000 mg, about 1 to about 600 mg, about 1.5 to about 300 mg, about 2 to about 100 mg, about 1 mg, about 2 mg, about 3 mg, about 5 mg, about 10 mg, about 20 mg, about 30 mg, about 40 mg, about 50 mg, about 100 mg, about 200 mg, about 300 mg, about 400 mg, about 500 mg, about 600 mg, about 1000 mg of one or more compounds disclosed herein in the form of tablets for oral administration.

The pharmaceutical compositions disclosed herein may be disclosed in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The compounds disclosed herein may be administered alone, or in combination with one or more other compounds disclosed herein, one or more other active ingredients. The pharmaceutical compositions that comprise a compound disclosed herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

### A. Oral Administration

The pharmaceutical compositions disclosed herein may be disclosed in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions disclosed herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions disclosed herein may be disclosed as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions disclosed herein may be disclosed as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions disclosed herein may be disclosed in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions disclosed herein for oral administration may be also disclosed in the forms of liposomes, micelles, microspheres, or nanosystems. Micellar dosage forms can be prepared as described in U.S. Pat. No. 6,350,458.

The pharmaceutical compositions disclosed herein may be disclosed as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions disclosed herein may be formulated as immediate or modified release dosage forms, including delayed-, sustained, pulsed-, controlled, targeted-, and programmed-release forms.

The pharmaceutical compositions disclosed herein may be co-formulated with other active ingredients which do not impair the desired therapeutic action, or with substances that supplement the desired action, such as drotrecogin-α, and hydrocortisone.

### B. Parenteral Administration

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are disclosed as ready-to-use sterile solutions. In another embodiment, the pharmaceutical compositions are disclosed as sterile dry soluble products, including lyophilized powders and hypodermic tablets, to be reconstituted with a vehicle prior to use. In yet another embodiment, the pharmaceutical compositions are disclosed as ready-to-use sterile suspensions. In yet another embodiment, the pharmaceutical compositions are disclosed as sterile dry insoluble products to be reconstituted with a vehicle prior to use. In still another embodiment, the pharmaceutical compositions are disclosed as ready-to-use sterile emulsions.

The pharmaceutical compositions disclosed herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions disclosed herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may also comprise liposomes, micelles, microspheres, nanosystems, and mixtures thereof.

Pharmaceutically acceptable carriers and excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be disclosed in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein may be administered intranasally or by inhalation to the respiratory tract. The pharmaceutical compositions may be disclosed in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be disclosed as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient disclosed herein, a propellant as solvent; and/or a surfactant, such as sorbitan trioleate, oleic acid, or an oligolactic acid.

The pharmaceutical compositions disclosed herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions disclosed herein; a suitable powder base, such as lactose or starch; and a performance modifier, such as l-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions disclosed herein for inhaled/intranasal administration may further comprise a suitable flavor, such as menthol and levomenthol, or sweeteners, such as saccharin or saccharin sodium.

The pharmaceutical compositions disclosed herein for topical administration may be formulated to be immediate release or modified release, including delayed-, sustained-, pulsed-, controlled-, targeted, and programmed release.

### D. Modified Release

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions disclosed herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions disclosed herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions disclosed herein in a modified release dosage form may be prepared by methods known to those skilled in the art, including direct compression, dry or wet granulation followed by compression, melt-granulation followed by compression.

### 2. Osmotic Controlled Release Devices

The pharmaceutical compositions disclosed herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions disclosed herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions disclosed herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions disclosed herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

The pharmaceutical compositions disclosed herein may also be formulated to be targeted to a particular tissue, enzyme, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

Disclosed are methods for treating, preventing, or ameliorating one or more symptoms of a PDE5-mediated disorder comprising administering to a subject having or being suspected to have such a disorder a therapeutically effective amount of a compound as disclosed herein or a pharmaceutically acceptable salt, solvate, or prodrug thereof.

Also disclosed are methods of treating, preventing, or ameliorating one or more symptoms of an PDE5-mediated disorder, by administering to a subject having or being suspected to have such a disorder, a therapeutically effective amount of a compound as disclosed herein or a pharmaceutically acceptable salt, solvate, or prodrug thereof.

Further disclosed are methods of treating, preventing, or ameliorating one or more symptoms of a disorder responsive to administering a PDE5 enzyme modulator, comprising administering to a subject having or being suspected to have such a disorder, a therapeutically effective amount of a compound as disclosed herein or a pharmaceutically acceptable salt, solvate, or prodrug thereof.

Furthermore, disclosed herein are methods of modulating the activity of PDE5 enzymes, comprising contacting the enzyme with at least one compound as disclosed herein or a pharmaceutically acceptable salt, solvate, or prodrug thereof. In one embodiment, the PDE5 enzyme is present in a cell.

Disclosed herein are methods for treating a subject, including a human, having or suspected of having a disorder, involving, but not limited to, hypertension, erectile dysfunction, the inability to maintain improved erectile function and/or any disorder which can be ameliorated, lessen, and/or attenuated by administering a phosphodiesterase type 5 enzyme modulator; comprising administering to the subject a therapeutically effective amount of a compound as disclosed herein or a pharmaceutically acceptable salt, solvate, or prodrug thereof; so as to affect decreased inter-individual variation in plasma levels of said compound or a metabolite thereof during treatment of the above-mentioned disorder as compared to the non-isotopically enriched compound. In another embodiment, the disorder is hypertension, erectile dysfunction, the inability to maintain improved erectile function. In yet another embodiment, the disorder is erectile dysfunction.

In certain embodiments, the inter-individual variation in plasma levels of the compounds as disclosed herein, or metabolites thereof, is decreased by greater than about 5%, greater than about 10%, greater than about 20%, greater than about 30%, greater than about 40%, or by greater than about 50% as compared to the corresponding non-isotopically enriched compound.

Disclosed herein are methods for treating a subject, including a human, having or suspected of having a disorder involving, but not limited to, hypertension, erectile dysfunction, the inability to maintain improved erectile function and/or any disorder which can be ameliorated, lessen, and/or attenuated by administering a phosphodiesterase type 5 enzyme modulator, in a subject prone to the disorder; comprising administering to the subject a therapeutically effective amount of a compound as disclosed herein or a pharmaceutically acceptable salt, solvate, or prodrug thereof, so as to affect increased average plasma levels of said compound or decreased average plasma levels of at least one metabolite of said compound per dosage unit as compared to the non-isotopically enriched compound.

In certain embodiments, the average plasma levels of the compound as disclosed herein are increased by greater than about 5%, greater than about 10%, greater than about 20%, greater than about 30%, greater than about 40%, or greater than about 50% as compared to the corresponding non-isotopically enriched compounds.

In certain embodiments, the average plasma levels of a metabolite of the compound as disclosed herein are decreased by greater than about 5%, greater than about 10%, greater than about 20%, greater than about 30%, greater than about 40%, or greater than about 50% as compared to the corresponding non-isotopically enriched compounds.

Plasma levels of the compounds as disclosed herein, or metabolites thereof, may be measured using the methods described by Li et al. (Rapid Communications in Mass Spectrometry 2005, 19, 1943-1950).

Disclosed herein are methods for treating a subject, including a human, having or suspected of having a disorder involving, but not limited to, hypertension, erectile dysfunction, the inability to maintain improved erectile function and/or any disorder which can be ameliorated, lessen, and/or attenuated by administering a phosphodiesterase type 5 enzyme modulator, or for preventing such disorder, in a subject prone to the disorder; comprising administering to the subject a therapeutically effective amount of a compound as disclosed herein or a pharmaceutically acceptable salt, solvate, or prodrug thereof, so as to affect a decreased inhibition of, and/or metabolism by at least one cytochrome P₄₅₀ or monoamine oxidase isoform in the subject during the treatment of the disorder as compared to the corresponding non-isotopically enriched compound.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}. In certain embodiments, the decrease in inhibition of the cytochrome P₄₅₀ or monoamine oxidase isoform by a compound as disclosed herein is greater than about 5%, greater than about 10%, greater than about 20%, greater than about 30%, greater than about 40%, or greater than about 50% as compared to the corresponding non-isotopically enriched compounds.

The inhibition of the cytochrome P₄₅₀ isoform is measured by the method of Ko et al. (British Journal of Clinical Pharmacology, 2000, 49, 343-351). The inhibition of the MAO_{A} isoform is measured by the method of Weyler et al. (J. Biol Chem. 1985, 260, 13199-13207). The inhibition of the MAO_{B} isoform is measured by the method of Uebelhack et al. (Pharmacopsychiatry, 1998, 31, 187-192).

Disclosed herein are methods for treating a subject, including a human, having or suspected of having a disorder involving, but not limited to, hypertension, erectile dysfunction, the inability to maintain improved erectile function and/or any disorder which can be ameliorated, lessen, and/or attenuated by administering a phosphodiesterase type 5 enzyme modulator, or for preventing such disorder, in a subject prone to the disorder; comprising administering to the subject a therapeutically effective amount of a compound as disclosed herein or a pharmaceutically acceptable salt, solvate, or prodrug thereof, so as to affect a decreased metabolism via at least one polymorphically-expressed cytochrome P₄₅₀ isoform in the subject during the treatment of the disorder as compared to the corresponding non-isotopically enriched compound.

Examples of polymorphically-expressed cytochrome P₄₅₀ isoforms in a mammalian subject include, but are not limited to, CYP2C8, CYP2C9, CYP2C19, and CYP2D6.

In certain embodiments, the decrease in metabolism of the compound as disclosed herein by at least one polymorphically-expressed cytochrome P₄₅₀ isoforms cytochrome P₄₅₀ isoform is greater than about 5%, greater than about 10%, greater than about 20%, greater than about 30%, greater than about 40%, or greater than about 50% as compared to the corresponding non-isotopically enriched compound.

The inhibition of the cytochrome P₄₅₀ isoform is measured by the methods of Ko et al., British Journal of Clinical Pharmacology, 2000, 49(4), 343-351, which is hereby incorporated by reference in its entirety. The inhibition of the MAO_{A} isoform is measured by the methods of Weyler et al., Journal of Biological Chemistry, 1985, 260(24), 13199-13207, which is hereby incorporated by reference in its entirety. The inhibition of the MAO_{B} isoform is measured by the methods of Uebelhack et al., Pharmacopsychiatry, 1998, 31(5), 187-192, which is hereby incorporated by reference in its entirety.

The metabolic activities of liver microsomes and the cytochrome P₄₅₀ isoforms are measured by the methods described in Examples 5. The metabolic activities of the monoamine oxidase isoforms are measured by the methods described in Examples 6, 7 and 8.

In another aspect of the invention, there are provided methods for treating a subject, particularly a human having, suspected of having, or being prone to a disorder involving, but not limited to, hypertension, erectile dysfunction, the inability to maintain improved erectile function and/or any disorder which can be ameliorated, lessen, and/or attenuated by administering a phosphodiesterase type 5 enzyme modulator, comprising administering to a subject in need thereof a therapeutically effective amount of a phosphodiesterase type 5 enzyme modulator comprising at least one of the compounds as disclosed herein or a pharmaceutically acceptable salt, solvate, or prodrug thereof, so as to affect an improved clinical effect comprising maintenance of clinical benefit (e.g., statistically-significantly improved disorder-control and/or disorder-eradication endpoints).

Disclosed herein are methods for treating a subject, including a human, having or suspected of having a disorder involving, but not limited to, hypertension, erectile dysfunction, the inability to maintain improved erectile function and/or any disorder which can be ameliorated, lessen, and/or attenuated by administering a phosphodiesterase type 5 enzyme modulator, or for preventing such disorder, in a subject prone to the disorder; comprising administering to the subject a therapeutically effective amount of a compound as disclosed herein or a pharmaceutically acceptable salt, solvate, or prodrug thereof; so as to affect at least one statistically-significantly improved disorder-control and/or disorder-eradication endpoint

Examples of a disorder-control and/or disorder-eradication endpoint include, but are not limited to, normalization of blood pressure, statistically significant improvement in the percentage of sexual attempts resulting in erections firm enough for intercourse, improved erections, improved erection hardness, improved partner satisfaction, and improvement in erectile dysfunction severity based on the International Index of Erectile Function.

Disclosed herein are methods for treating a subject, including a human, having or suspected of having a disorder involving, but not limited to, hypertension, erectile dysfunction, the inability to maintain improved erectile function and/or any disorder which can be ameliorated, lessen, and/or attenuated by administering a phosphodiesterase type 5 enzyme modulator, or for preventing such disorder, in a subject prone to the disorder; comprising administering to the subject a therapeutically effective amount of a compound as disclosed herein or a pharmaceutically acceptable salt, solvate, or prodrug thereof, so as to affect an improved clinical effect as compared to the corresponding non-isotopically enriched compound. Examples of improved clinical effects include, but are not limited to, normalization of blood pressure, statistically significant improvements in patient satisfaction as measured by their responses to a Sexual Encounter Profile (SEP) diary questionnaire (questions directed to: successful intercourse, improved erections, partner satisfaction, successful penetration, among others), and improvement in erectile dysfunction severity based on the International Index of Erectile Function.

Disclosed herein are methods for treating a subject, including a human, having or suspected of having a disorder involving, but not limited to, hypertension, erectile dysfunction, the inability to maintain improved erectile function and/or any disorder which can be ameliorated, lessen, and/or attenuated by administering a phosphodiesterase type 5 enzyme modulator, or for preventing such disorder, in a subject prone to the disorder; comprising administering to the subject a therapeutically effective amount of a compound as disclosed herein or a pharmaceutically acceptable salt, solvate, or prodrug thereof; so as to affect prevention of recurrence, or delay of decline or appearance, of abnormal alimentary or hepatic parameters as the primary clinical benefit, as compared to the corresponding non-isotopically enriched compound.

Disclosed herein are methods for treating a subject, including a human, having or suspected of having a disorder involving, but not limited to, hypertension, erectile dysfunction, the inability to maintain improved erectile function and/or any disorder which can be ameliorated, lessen, and/or attenuated by administering a phosphodiesterase type 5 enzyme modulator, or for preventing such disorder, in a subject prone to the disorder; comprising administering to the subject a therapeutically effective amount of a compound as disclosed herein or a pharmaceutically acceptable salt, solvate, or prodrug thereof, so as to allow the treatment hypertension, erectile dysfunction, the inability to maintain improved erectile function and/or any disorder which can be ameliorated, lessen, and/or attenuated by administering a phosphodiesterase type 5 enzyme modulator, while reducing or eliminating deleterious changes in any diagnostic hepatobiliary function endpoints as compared to the corresponding non-isotopically enriched compound.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Depending on the disorder to be treated and the subject's condition, the compound as disclosed herein disclosed herein may be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.1 to about 1000 milligram, from about 0.2 to about 600 milligram, from about 0.3 to about 300 milligram, from about 0.4 to 200 milligram, from about 0.5 to about 150 milligram, from about 0.6 to 100 milligram active ingredient(s) per dosage unit, and if the condition of the patient requires, the dose can, by way of alternative, be administered as a continuous infusion.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

### Combination Therapy

The compounds disclosed herein may also be combined or used in combination with other agents useful in the treatment, prevention, or amelioration of one or more symptoms of, but not limited to, hypertension, erectile dysfunction, the inability to maintain improved erectile function and/or any disorder which can be ameliorated, lessen, and/or attenuated by administering a phosphodiesterase type 5 enzyme modulator. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required. Accordingly, the pharmaceutical compositions disclosed herein include those that also contain one or more other active ingredients or therapeutic agents, in addition to the compound disclosed herein.

In certain embodiments, the compounds provided herein can be combined with one or more phosphodiesterase 5 inhibitors known in the art, including, but not limited to, sildenafil, tadalafil, vardenafil, udenafil and avanafil.

In other embodiments, the compounds provided herein can be combined with one or more experimental erectile dysfunction treatments, including, but not limited to, Ginko Biloba extract, Saw Palmetto extract, Muira Pauma extract, Prelox°, Tribulus terrestris extract, Lepidium meyenii extract, Epimediu extract, Ginseng, bremelanotide, Naltrexone, Melanotan II, and Zinc.

In certain embodiments, the compounds provided herein can be combined with one or more CYP3A inhibitors known in the art, including, but not limited to, fluconazole, ritonavir, macrolide antibiotics, azole antifungals, nefazodone, bergamottin, amiodarone, aprepitant, cimetidine, ciprofloxacin, ciclosporin, diltiazem, imatinib, Echinacea, enoxacin, ergotamine, metronidazole, mifepristone, efavirenz, nevirapine, gestodene, mibefradil, fluoxetine, and verapamil.

In certain embodiments, the compounds provided herein can be combined with one or more CYP3A inducers known in the art, including, but not limited to, barbiturates, hyperforin, non-nucleoside reverse transcriptase inhibitors, phenyloin, rifampicin, dexamethasone, felbamate, glucocorticoids, griseofulvin, pioglitazone, primidone, topiramate, troglitazone, and rifabutin.

In certain embodiments, the compounds provided herein can be combined with one or more protease inhibitors known in the art, including, but not limited to, amprenavir, atazanavir, darunavir, fosamprenavir, indinavir, lopinavir, nelfinavir, ritonavir, saquinavir, and tipranavir

In certain embodiments, the compounds provided herein can be combined with one or more antibacterial agents known in the art, including, but not limited to, amikacin, p-aminosalisylic acid, amoxicillin, ampicillin, arsphenamine, azithromycin, aztreonam, azlocillin, bacitracin, capreomycin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clindamycin, clofazimine, cloxacillin, colistin, cycloserine, dalfopristan, demeclocycline, dicloxacillin, dirithromycin, doxycycline, erythromycin, enafloxacin, enviomycin, ertepenem, ethambutol, ethionamide, flucloxacillin, fosfomycin, furazolidone, gatifloxacin, geldanamycin, gentamicin, herbimicin, imipenem, isoniazide, kanamicin, levofloxacin, linezolid, lomefloxacin, loracarbef, mafenide, moxifloxacin, meropenem, metronidazole, mezlocillin, minocycline, mupirozin, nafcillin, neomycin, netilmicin, nitrofurantoin, norfloxacin, ofloxacin, oxytetracycline, penicillin, piperacillin, platensimycin, polymixin B, prochlorperazine, prontocil, prothionamide, pyrazinamide, quinupristine, rifabutin, rifampin, roxithromycin, spectinomycin, streptomycin, sulfacetamide, sulfamethizole, sulfamethoxazole, teicoplanin, telithromycin, tetracycline, thioacetazone, thioridazine, ticarcillin, tobramycin, trimethoprim, troleandomycin, trovafloxacin, vancomycin and viomycin.

In certain embodiments, the compounds disclosed herein can be combined with one or more antifungal agents known in the art, including, but not limited to the group including amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole.

In certain embodiments, the compounds disclosed herein can be combined with one or more sepsis treatments known in the art, including, but not limited to drotrecogin-α or a biosimilar of activated protein C.

In certain embodiments, the compounds disclosed herein can be combined with one or more steroidal drugs known in the art, including, but not limited to, aldosterone, beclometasone, betamethasone, deoxycorticosterone acetate, fludrocortisone acetate, hydrocortisone (cortisol), prednisolone, prednisone, methylprenisolone, dexamethasone, and triamcinolone.

In certain embodiments, the compounds disclosed herein can be combined with one or more anticoagulants known in the art, including, but not limited to the group including acenocoumarol, argatroban, bivalirudin, lepirudin, fondaparinux, heparin, phenindione, warfarin, and ximalagatran.

In certain embodiments, the compounds disclosed herein can be combined with one or more thrombolytics known in the art, including, but not limited to the group including anistreplase, reteplase, t-PA (alteplase activase), streptokinase, tenecteplase, and urokinase.

In certain embodiments, the compounds disclosed herein can be combined with one or more non-steroidal anti-inflammatory agents known in the art, including, but not limited to the group including aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

In certain embodiments, the compounds disclosed herein can be combined with one or more antiplatelet agents known in the art, including, but not limited to the group including abciximab, cilostazol, clopidogrel, dipyridamole, ticlopidine, and tirofibin.

The compounds disclosed herein can also be administered in combination with other classes of compounds, including, but not limited to, endothelin converting enzyme (ECE) inhibitors, such as phosphoramidon; thromboxane enzyme antagonists, such as ifetroban; potassium channel openers; thrombin inhibitors, such as hirudin; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-PDE5 agents; beta-PDE5 agents, such as carvedilol and metoprolol; antiarrhythmic agents; diuretics, such as chlorothlazide, hydrochlorothiazide, flumethiazide, hydroflumethiazide, bendroflumethiazide, methylchlorothiazide, trichloromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid enzyme antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF enzyme, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

### Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic. For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

For example, certain compounds as disclosed herein can be prepared as shown in Scheme 1.

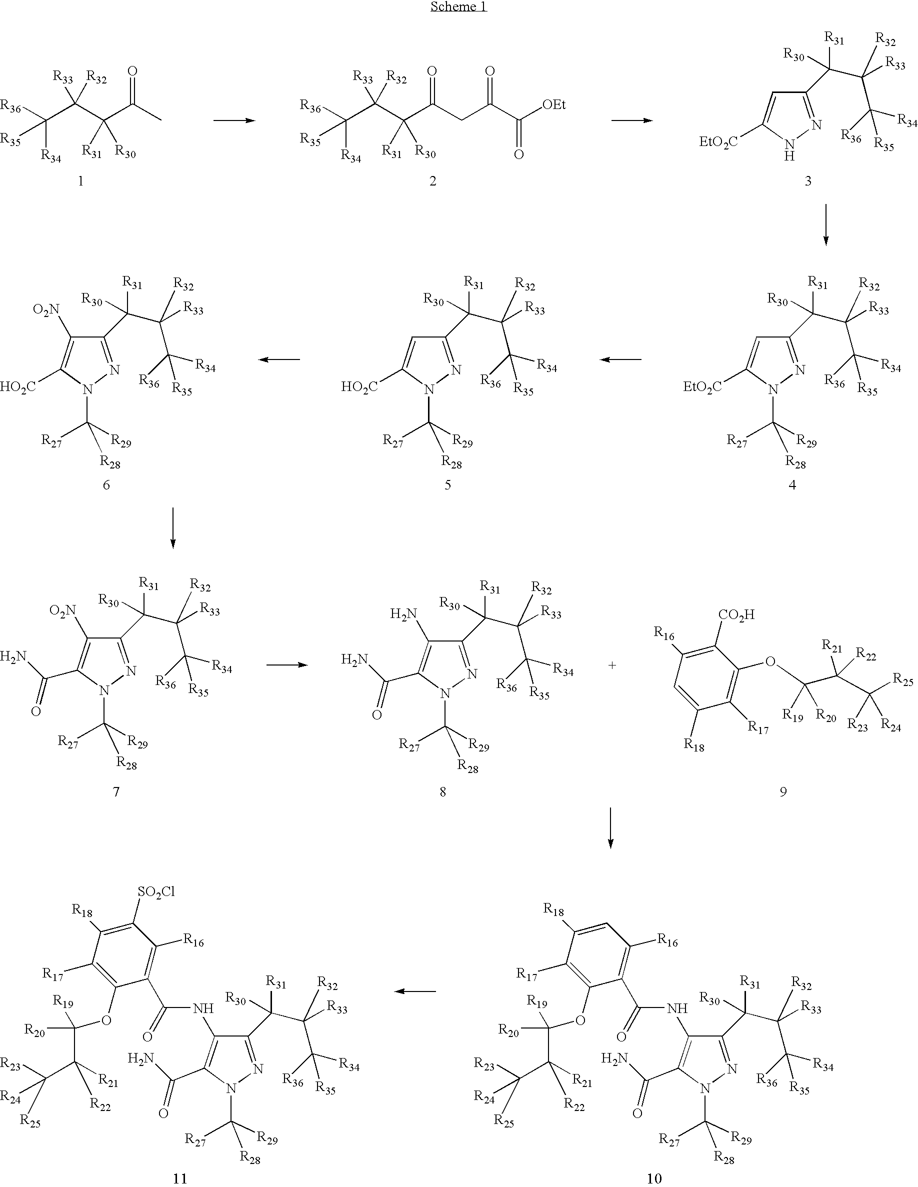

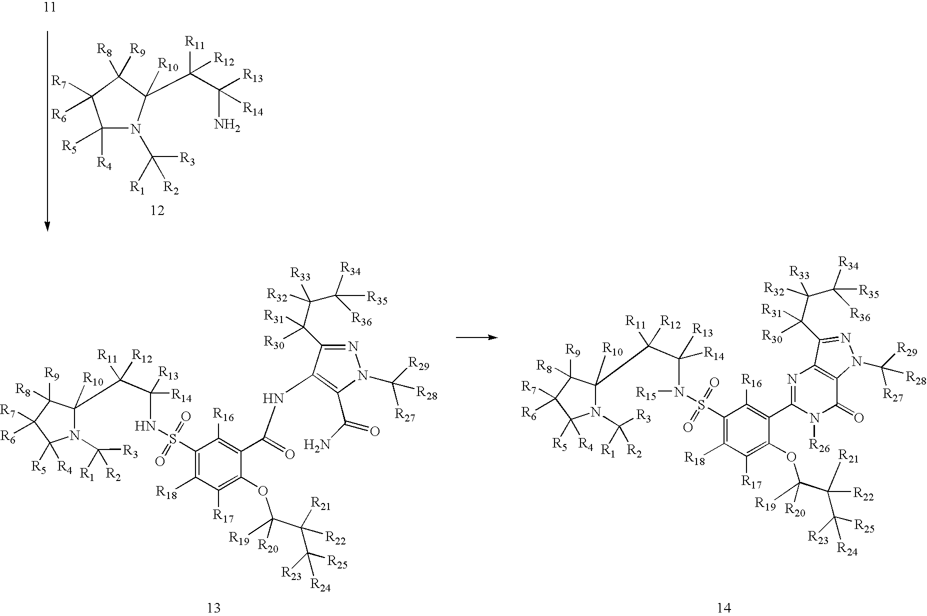

Ketone 1 is treated with an appropriate base, such as sodium, in an appropriate solvent, such as ethanol, and reacted with diethyl oxalate to give trione 2 which is subsequently reacted with hydrazine hydrate in an appropriate solvent, such as acetic acid, at an elevated temperature to give pyrazole ester 3. Compound 3 is reacted with an appropriate methylating agent, such as dimethyl sulfate, at an elevated temperature, to give methylpyrazole 4, which is treated with an appropriate base, such as sodium hydroxide, at an elevated temperature, to give pyrazole acid 5. Compound 5 is treated with an appropriate acid, such as nitric acid or sulfuric acid or an appropriate mixture thereof, at an elevated temperature, to give nitro pyrazole 6, which is treated with thionyl chloride, dimethylforamide and ammonium hydroxide in an appropriate solvent, such as toluene, at an elevated temperature to give pyrazole amide 7. Compound 7 is reacted with hydrogen in the presence of an appropriate catalyst, such as 10% palladium on carbon, in an appropriate solvent, such as ethyl acetate, at an elevated temperature and pressure to give amino pyrazole 8, which is reacted with benzoic acid 9 and thionyl chloride in an appropriate solvent, such as dichloromethane, in the presence of an appropriate base, such as triethylamine or 4-(N,N-dimethylamino)pyridine or an appropriate mixture thereof, to give amide 10. Compound 10 is treated with an appropriate sulfonating agent, such as chlorosulfonic acid, to give sulfonyl chloride 11, which is treated with amine 12 in the presence of an appropriate base, such as triethylamine, in an appropriate solvent, such as dichloromethane, to give sulfonamide 13. Compound 13 is treated with an appropriate base, such a potassium tert-butoxide, in an appropriate solvent, such as ter-butanol, at an elevated temperature to give pyrazolo-pyrimidinone 14 of Formula (I).

Deuterium can be incorporated to different positions synthetically, according to the synthetic procedures as shown in Scheme 1, by using appropriate deuterated intermediates. For example, to introduce deuterium at one or more positions of R₁, R₂, R₃, R₄, R₅, R₆, R₇, R₈, R₉, R₁₁, R₁₂, R₁₃, and R₁₄, 2-(2-aminoethyl)-1-methylpyrrolidine with the corresponding deuterium substitutions can be used. To introduce deuterium at one or more positions of R₁₆, R₁₇, R₁₈, R₁₉, R₂₀, R₂₁, R₂₂, R₂₃, R₂₄, and R₂₅, 2-propoxybenzoic acid with the corresponding deuterium substitutions can be used. To introduce deuterium at one or more positions of R₂₇, R₂₈, and R₂₉, deuterated dimethylsulfate can be used. To introduce deuterium at one or more positions of R₃₀, R₃₁, R₃₂, R₃₃, R₃₄, R₃₅, and R₃₆, 2-pentanone with the corresponding deuterium substitutions can be used. These deuterated intermediates are either commercially available, or can be prepared by methods known to one of skill in the art or following procedures similar to those described in the Example section herein and routine modifications thereof.

Deuterium can also be incorporated to various positions having an exchangeable proton, such as the sulfonamide and pyrazolo-pyrimidinone N—H, via proton-deuterium equilibrium exchange. For example, to introduce deuterium at R₁₅, and R₂₆ these protons may be replaced with deuterium selectively or non-selectively through a proton-deuterium exchange method known in the art.

The invention is further illustrated by the following examples.

### EXAMPLE 1

### d₃-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide (d₃-udenafil)

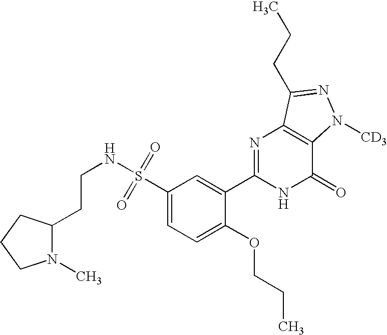

### Step 1

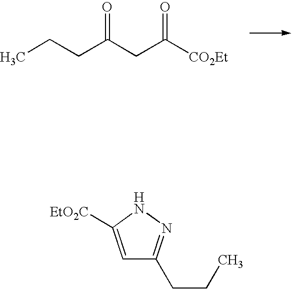

5-Propyl-2H-pyrazole-3-carboxylic acid ethyl ester: This process is carried out according to van Herk et al Journal of Medicinal Chemistry, 2003, 46(18), 3945-3951, which is hereby incorporated by reference in its entirety. Hydrazine hydrate (29.56 mmol) is slowly added to a solution of 2,4-dioxo-heptanoic acid ethyl ester (26.85 mmol) in 10 mL of acetic acid at 0° C. The mixture is heated to reflux for 8 hours and cooled. The resulting solid is filtered and dried in vacuo, to give the desired product, 5-propyl-2H-pyrazole-3-carboxylic acid ethyl ester, as white crystals.

### Step 2

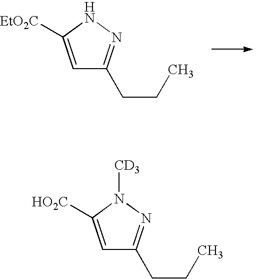

d₃-2-Methyl-5-propyl-2H-pyrazole-3-carboxylic acid: This process is carried out according to EP 463,756, which is hereby incorporated by reference in its entirety. A mixture of 5-propyl-2H-pyrazole-3-carboxylic acid ethyl ester (0.132 mmol) and d₆-dimethyl sulfate (0.133 mmol) is heated at 90° C. for 3 hours. The reaction is cooled, diluted with dichloromethane and washed with aqueous sodium bicarbonate. The organic layer is dried over anhydrous magnesium sulfate, filtered and concentrated under reduced pressure to give a crude residue which is purified by flash chromatography (dichloromethane eluant) to give d₃-2-methyl-5-propyl-2H-pyrazole-3-carboxylic acid ethyl ester, as a colorless oil.

d₃-2-Methyl-5-propyl-2H-pyrazole-3-carboxylic acid ethyl ester (0.1 mmol) is suspended in 6N sodium hydroxide (0.3 mmol) and heated to 80° C. for 2 hours, cooled, diluted with water and acidified with concentrated HCl to give a precipitate which is filtered off and dried to give the desired product, d₃-2-methyl-5-propyl-2H-pyrazole-3-carboxylic acid, as pale brown crystals.

### Step 3

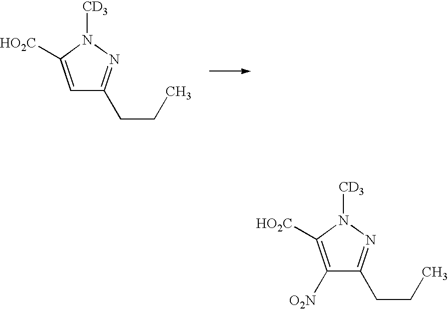

d₃-2-Methyl-4-nitro-5-propyl-2H-pyrazole-3-carboxylic acid: This process is carried out according to Dale et al Organic Process Research & Development 2000, 4(1), 17-22, which is hereby incorporated by reference in its entirety. A solution of d₃-1-methyl-3-propyl-1H-pyrazole-5-carboxylic acid (9.45 mmol) in concentrated sulfuric acid (6.36 mL) is heated to 50° C. and treated with a mixture of fuming nitric acid (90%, 0.55 mL) and concentrated sulfuric acid (1.35 mL), while keeping the reaction temperature between 50 and 55° C. The reaction is kept for 8 hours at 50° C., cooled to room temperature, and slowly added to cold water (34 mL, 4° C.), keeping the temperature below 25° C. The precipitate is collected by filtration, and dried in vacuo at 50° C. to give the desired product, d₃-2-methyl-4-nitro-5-propyl-2H-pyrazole-3-carboxylic acid, as a pale yellow solid.

### Step 4

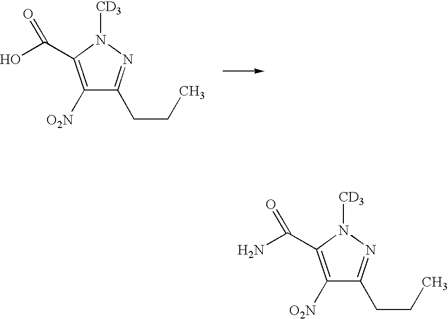

d₃-2-Methyl-4-nitro-5-propyl-2H-pyrazole-3-carboxylic acid amide: This process is carried out according to Dale et al Organic Process Research & Development 2000, 4(1), 17-22, which is hereby incorporated by reference in its entirety. To a slurry of d₃-2-methyl-4-nitro-5-propyl-2H-pyrazole-3-carboxylic acid (4.69 mmol) in toluene (5 mL) is added a catalytic quantity of dimethylformamide (37 μL). The mixture is heated to 50° C. and thionyl chloride (7.5 mmol) is added over 10 min. The reaction is stirred and heated at 55-60° C. for 6 hours. The solvent is removed, toluene is added and the mixture is cooled to 20° C. and cold (5° C.) concentrated ammonium hydroxide (6 mL) is added. The precipitate is filtered, washed with water and dried at 50° C. to give the desired product, d₃-2-methyl-4-nitro-5-propyl-2H-pyrazole-3-carboxylic amide, as an off white solid.

### Step 5

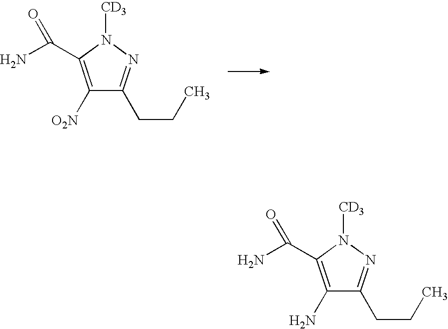

d₃-4-Amino-2-methyl-5-propyl-2H-pyrazole-3-carboxylic acid amide: This process is carried out according to Dale et al, Organic Process Research & Development 2000, 4(1), 17-22, which is hereby incorporated by reference in its entirety. To a suspension of d₃-2-methyl-4-nitro-5-propyl-2H-pyrazole-3-carboxylic amide (1.12 mmol) in ethyl acetate (2.02 mL), is added 5% palladium on carbon (2 mol %). The resulting mixture is hydrogenated at 50° C. and 50 psi for 4 hours. The reaction is cooled, and the catalyst is filtered off and washed with ethyl acetate. The solvent is removed to give the desired product, d₃-4-amino-2-methyl-5-propyl-2H-pyrazole-3-carboxylic acid amide, which is used directly in the next step.

### Step 6

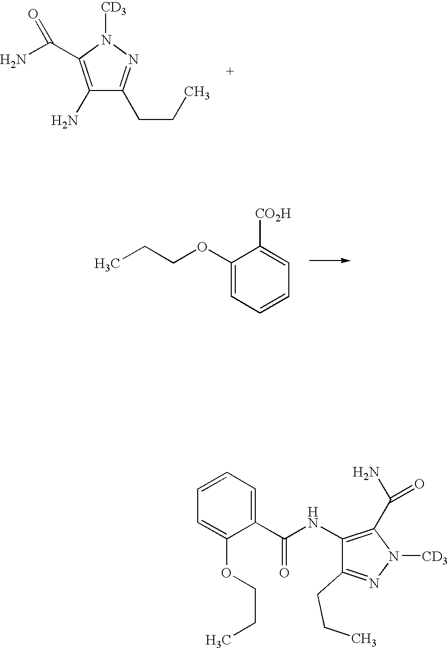

d₃-2-Methyl-4-(2-propoxybenzoylamino)-5-propyl-2H-pyrazole-3-carboxylic acid amide: This process is carried out according to U.S. Pat. No. 6,844,436, which is hereby incorporated by reference in its entirety. A solution of 2-propoxybenzoic acid (1.4 mmol) and thionyl chloride (5.6 mmol) in dichloromethane is heated for 3 hours under reflux. The solvent and excessive thionyl chloride are distilled off under reduced pressure. The residue is taken up in dichloromethane and reacted with a solution of d₃-4-amino-2-methyl-5-propyl-2H-pyrazole-3-carboxylic acid amide (1.32 mmol), triethylamine (1.33 mmol) and dimethylaminopyridine (0.01 mmol) in dichloromethane at 0° C. Stirring is maintained for 1 hour, and the reaction mixture is successively washed with water, saturated aqueous sodium bicarbonate solution and brine. The organic layer is dried over anhydrous sodium sulfate and filtered. The filtrate is concentrated under reduced pressure to give a crude residue which is triturated with hexane to give the desired product, d₃-2-methyl-4-(2-propoxybenzoylamino)-5-propyl-2H-pyrazole-3-carboxylic acid amide.

### Step 7

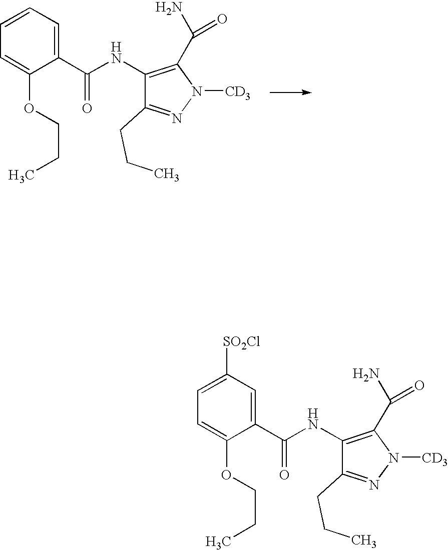

d₃-3-(5-Carbamoyl-1-methyl-3-propyl-1H-pyrazol-4-ylcarbamoyl)-4-propoxy-benzenesulfonyl chloride: This process is carried out according to U.S. Pat. No. 6,844,436, which is hereby incorporated by reference in its entirety. d₃-2-Methyl-4-(2-propoxybenzoylamino)-5-propyl-2H-pyrazole-3-carboxylic acid amide (10 g) is added to chlorosulfonic acid (23 mL) at 0° C. and the reaction is warmed to ambient temperature and stirred for 2 hours. The reaction mixture is poured into ice water and stirred for 1 hour to give a white solid, which is filtered and washed with water. The white solid is dissolved in ethyl acetate, and washed with water and brine. The organic layer is dried over anhydrous magnesium sulfate, filtered and concentrated under reduced pressure to give a crude residue which is triturated with hexane to give the desired product, d₃-3-(5-carbamoyl-1-methyl-3-propyl-1H-pyrazol-4-ylcarbamoyl)-4-propoxy-benzenesulfonyl chloride.

### Step 8

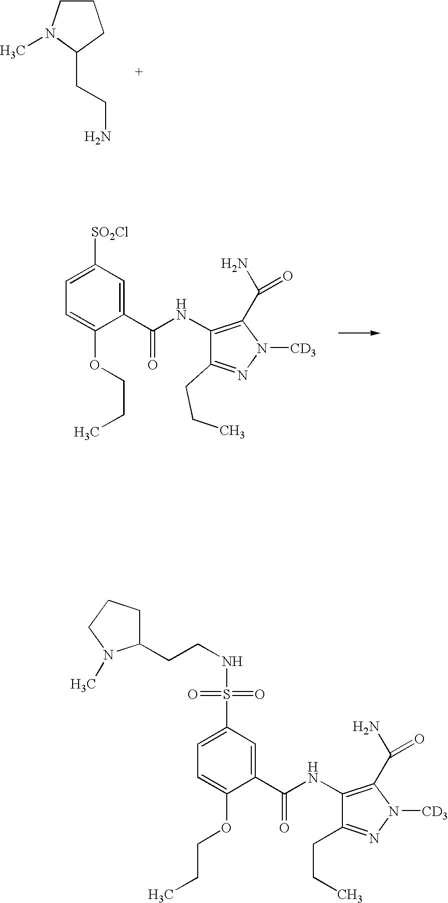

d₃-2-Methyl-4-{5-[2-(1-methyl-pyrrolidin-2-yl)-ethylsulfamoyl]-2-propoxy-benzoylamino}-5-propyl-2H-pyrazole-3-carboxylic acid amide: This process is carried out according to Dale et al Organic Process Research & Development 2000, 4(1), 17-22, which is hereby incorporated by reference in its entirety. To a suspension of d₃-2-methyl-4-nitro-5-propyl-2H-pyrazole-3-carboxylic amide (1.12 mmol) in ethyl acetate (2.02 mL), is added 5% palladium on carbon (2 mol %). The resulting mixture is hydrogenated at 50° C. and 50 psi for 4 hours. The reaction is cooled, and the catalyst is filtered off and washed with ethyl acetate. The solvent is removed to give the desired product, d₃-4-amino-2-methyl-5-propyl-2H-pyrazole-3-carboxylic acid amide, which is used directly in the next step.

### Step 9

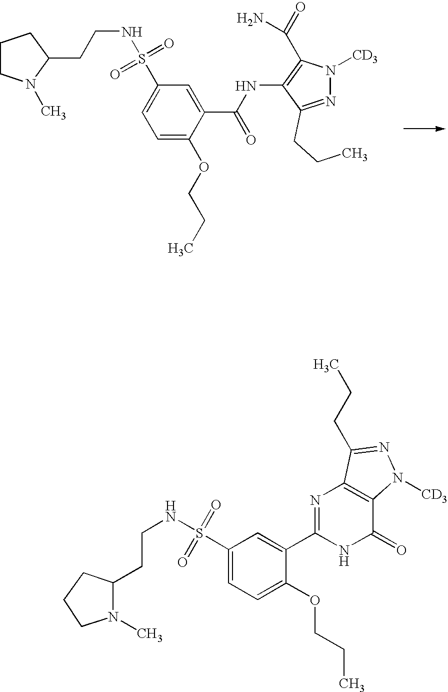

d₃-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide (d₃-udenafil): This process is carried out according to U.S. Pat. No. 6,844,436, which is hereby incorporated by reference in its entirety. To a solution of d₃-2-methyl-4-{5-[2-(1-methyl-pyrrolidin-2-yl)-ethylsulfamoyl]-2-propoxy-benzoylamino}-5-propyl-2H-pyrazole-3-carboxylic acid amide (9.59 g) in tert-butanol (200 mL), is added potassium tert-butoxide (4 g) and the mixture is heated to reflux for 8 hours. The reaction is cooled to ambient temperature, diluted with ethyl acetate, washed with water and brine. The organic layer is dried over anhydrous magnesium sulfate, filtered and the solvent is removed. The crude residue is purified by flash chromatography to give the desired product, d₃-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide (d₃-udenafil).

### EXAMPLE 2

### 3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide

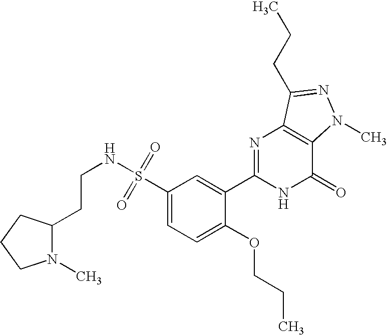

### Step 1

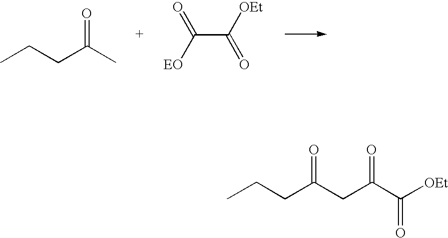

2,4-Dioxo-heptanoic acid methyl ester: Sodium (25.3 g, 1.1 mol) was proportionally added to ethanol (350 mL) at ambient temperature with vigorous stirring, and the solution was cooled to 0° C. Pentan-2-one (86 g, 1.0 mol) and diethyl oxalate (146 g, 1.0 mol) were added sequentially at 0° C., and stirring was continued for 1 hour at 0° C., and overnight at ambient temperature. The solvent was removed under reduced pressure, diethyl ether (200 mL) and cold dilute hydrochloric acid (500 mL) were added. Following standard extractive work up, the solvent was evaporated under reduced pressure to yield the title compound (141 g, 76%). ¹H-NMR (300 MHz, CDCl3) δ 14.51 (broad s, 1H), 6.37 (s, 1H), 4.35 (q, 2H, J=6.6 Hz), 2.47 (t, 2H, J=7.2 Hz), 1.76-1.66 (m, 2H), 1.38 (t, 3H, J=7.2 Hz), 0.97 (t, 3H, J=7.5 Hz); GC-MS: 186 (M)⁺, 113 (M-73)⁺

### Step 2

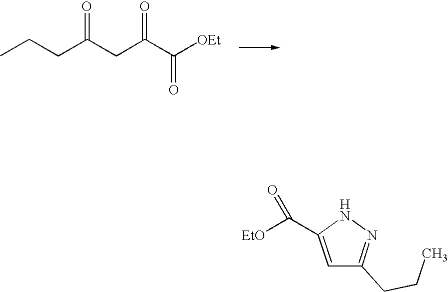

5-Propyl-2H-pyrazole-3-carboxylic acid ethyl ester: Hydrazine hydrate (41.4 g, 827 mmol) was slowly added to a solution of 2,4-dioxo-heptanoic acid methyl ester (140 g, 753 mmol) in 280 mL of acetic acid at 0° C. The mixture was heated to reflux for 8 hours and cooled. The solvent was removed under reduced pressure; the residue was diluted with diethyl ether (300 mL). Following standard extractive work up, the solvent was evaporated under reduced pressure to yield the title compound as a white solid (131 g, 96%). ¹H NMR (300 MHz, CDCl₃) δ 9.27 (broad s, 1H), 6.61 (s, 1H), 4.37 (q, 2H, J=7.2 Hz), 2.68 (t, 2H, J=7.5 Hz), 1.75-1.62 (m, 2H), 1.37 (t, 3H, J=6.6 Hz), 0.96 (t, 3H, J=7.2 Hz); LC-MS: m/z=183 (MH)⁺;

### Step 3

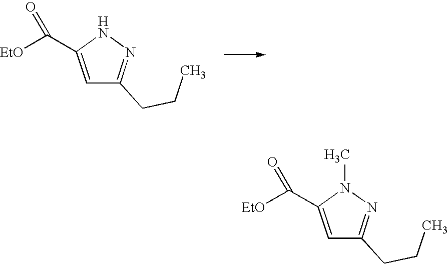

2-Methyl-5-propyl-2H-pyrazole-3-carboxylic acid ethyl ester: A mixture of 5-propyl-2H-pyrazole-3-carboxylic acid ethyl ester (32.8 g, 180 mmol) and dimethyl sulfate (24.9 g, 198 mmol) was heated at 90° C. for 3 hours. The reaction was cooled and diluted with dichloromethane (200 mL). Following standard extractive work up, the solvent was evaporated under reduced pressure to yield a crude residue which was purified by flash chromatography on silica gel to give the title compound as a colorless oil (23 g, 65%). ¹H NMR (300 MHz, CDCl₃) δ 6.59 (s, 1H), 4.37 (q, 2H, J=7.2 Hz), 2.58 (t, 2H, J=7.2 Hz), 1.76-1.64 (m, 2H), 1.40 (t, 3H, J=6.6 Hz), 1.01 (t, 3H, J=7.2 Hz), 4.40 (q, 2H), 3.89 (s, 3H), 2.59 (t, 2H), 1.69 (2H), 1.37 (t, 3H), 1.01 (t, 3H); LC-MS: m/z=197 (MH)⁺.

### Step 4

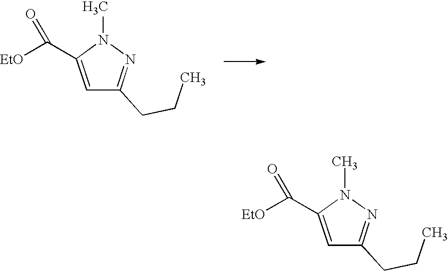

2-Methyl-5-propyl-2H-pyrazole-3-carboxylic acid: 2-methyl-5-propyl-2H-pyrazole-3-carboxylic acid ethyl ester (29.4 g, 150 mmol) was suspended in 6N sodium hydroxide (120 mL, 720 mmol) and heated to 80° C. for 2 hours, cooled, diluted with water (100 mL) and acidified with 5N hydrochloric acid (200 mL) to give a precipitate which was filtered off and dried to give the title compound as a white solid (24.2 g, 96%). ¹H NMR (300 MHz, CDCl₃) δ 6.76 (s, 1H), 4.17 (s, 3H), 2.63 (t, 2H, J=7.2 Hz), 1.70-1.68 (m, 2H), 0.98 (t, 3H, J=7.2 Hz); LC-MS: m/z=169 (M+H)⁺;

### Step 5

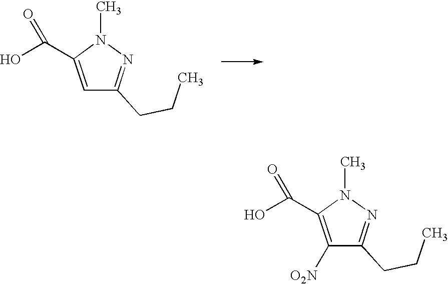

2-Methyl-4-nitro-5-propyl-2H-pyrazole-3-carboxylic acid: A solution of 2-methyl-5-propyl-2H-pyrazole-3-carboxylic acid (22 g, 131 mmol) in concentrated sulfuric acid (98%, 85 mL) was heated to 50° C. and treated with a mixture of fuming nitric acid (95%, 7.7 mL) and concentrated sulfuric acid (98%, 18 mL), while keeping the reaction temperature between 50 and 55° C. The reaction mixture was kept for 8 hours at 50° C., cooled to ambient temperature, and slowly added to cold water (600 mL, 4° C.), keeping the temperature below 25° C. The precipitate was collected by filtration, and dried below 80° C. to give the title compound as a white solid (25 g, 90%). ¹H NMR (300 MHz, CDCl₃) δ 4.25 (s, 3H), 2.92 (t, 2H, J=7.5 Hz), 1.77-1.70 (m, 2H), 1.03 (t, 3H, J=7.2 Hz); LC-MS: m/z=214 (M+H)⁺

### Step 6

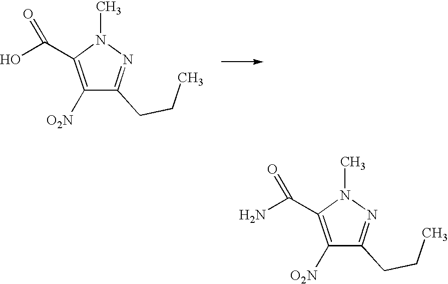

2-Methyl-4-nitro-5-propyl-2H-pyrazole-3-carboxamide: To a suspension of 2-methyl-4-nitro-5-propyl-2H-pyrazole-3-carboxylic acid (17.0 g, 79.8 mmol) in dry toluene (85 mL) was added a catalytic quantity of dimethylformamide (0.6 mL). The mixture was heated to 50° C. and thionyl chloride (17.1 g, 143.7 mmol) was added over 30 minutes. The reaction was stirred and heated at 55-60° C. for 6 hours. The solvent was removed, dry toluene (80 mL) was added and the mixture was cooled to 20° C. and cold (5° C.) concentrated ammonium hydroxide (100 mL) was added. The precipitate was filtered, washed with water and dried to give the title compound as an off-white solid (14.8 g, 87%). LC-MS: m/z=213 (M+H)⁺, 235 (M+Na)⁺.

### Step 7

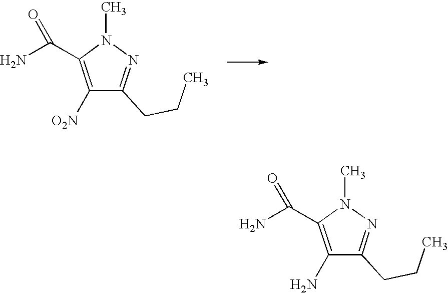

4-Amino-2-methyl-5-propyl-2H-pyrazole-3-carboxamide: To a suspension of 2-methyl-4-nitro-5-propyl-2H-pyrazole-3-carboxamide (14.7 g, 69.3 mmol) in ethyl acetate (130 mL), was added 10% palladium on carbon (3.3 g). The mixture was reacted at 50° C. and 4 atm hydrogen pressure overnight. The reaction mixture was cooled, and the catalyst was filtered off and washed with ethyl acetate and dried over anhydrous magnesium sulfate, filtered and concentrated under reduced pressure to give the title compound (13.8 g, 98%). ¹H NMR (300 MHz, CDCl₃) δ 4.12 (s, 3H), 2.84 (s, 2H), 2.55 (t, 2H, J=7.2 Hz), 1.71-1.61 (m, 2H), 0.99 (t, 3H, J=7.2 Hz); LC-MS: m/z=183 (MH)⁺

### Step 8

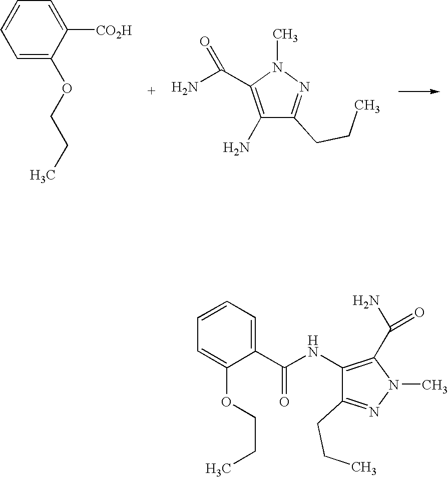

2-Methyl-4-(2-propoxybenzoylamino)-5-propyl-2H-pyrazole-3-carboxamide: A solution of 2-propoxybenzoic acid (13.7 g, 76.1 mmol) and thionyl chloride (36.2 g, 304.4 mmol) in dry dichloromethane (80 mL) was heated for 3 hours at reflux. The solvent and excess thionyl chloride were distilled off under reduced pressure. The residue was taken up in dry dichloromethane (60 mL) and reacted with a solution of 4-amino-2-methyl-5-propyl-2H-pyrazole-3-carboxamide (12.6 g, 69.2 mmol), dry triethylamine (7 g, 69.2 mmol) and 4-(N,N-dimethylamino)pyridine (84.5 mg, 0.7 mmol) in dry dichloromethane (200 mL) at 0° C. Stirring was maintained for 1 hour, and the reaction mixture was successively washed with water (150 mL), saturated aqueous sodium carbonate solution (200 mL) and saturated brine (200 mL). The organic layer was dried over anhydrous magnesium sulfate and filtered. The filtrate was concentrated to about 60 mL, and then hexane (150 mL) was added to give precipitate product as a white solid (22 g, 92%). ¹H NMR (300 MHz, CDCl₃) δ 9.47 (s, 1H), 8.28 (d, 1H, J=7.8 Hz), 7.87 (br.s, 1H), 7.57-7.52 (m, 1H), 7.16-7.05 (m, 2H), 5.53 (s, 1H), 4.20 (t, 2H, J=6.6 Hz), 4.09 (s, 3H), 2.54 (t, 2H, J=7.5 Hz), 1.97-1.85 (m, 2H), 1.69-1.26 (m, 2H), 1.07 (t, 3H, J=7.2 Hz), 0.95 (t, 3H, J=7.5 Hz). LC-MS: m/z=345 (M+H)⁺

### Step 9

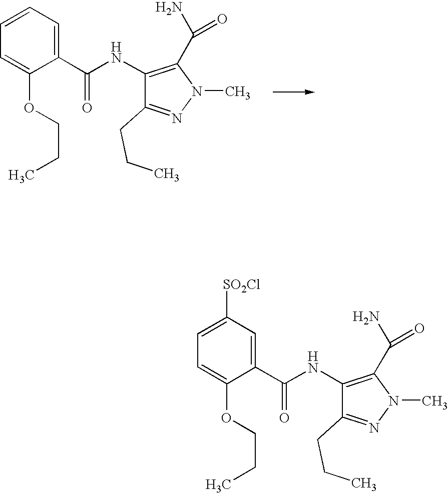

3-(5-Carbamoyl-1-methyl-3-propyl-1H-pyrazol-4-ylcarbamoyl)-4-propxy-benzenesulfonyl chloride: 2-Methyl-4-(2-propoxybenzoylamino)-5-propyl-2H-pyrazole-3-carboxamide (20 g, 58.1 mmol) was added to chlorosulfonic acid (81.3 g, 698 mmol) at 0° C. and the reaction was warmed to ambient temperature and stirred for 2 hours. The reaction mixture was poured into ice water (800 g) and mechanically stirred for 1 hour to give a white solid, which was filtered and washed with water. Following standard extractive work up, the solvent was evaporated under reduced pressure to yield the title compound (8 g, 31%). ¹H NMR (300 MHz, CDCl₃) δ 9.19 (s, 1H), 8.97 (s, 1H), 8.19 (t, 1H, J=8.9 Hz), 7.56 (br. s, 1H), 4.35 (t, 2H, J=6.6 Hz), 4.07 (s, 3H), 2.53 (t, 2H, J=7.5 Hz), 2.06-1.94 (m, 2H), 1.78-1.60 (m, 2H), 1.18 (t, 3H, J=7.5 Hz), 0.95 (t, 3H, J=7.2 Hz); LC-MS: m/z=443.1 (M+H)⁺

### Step 10

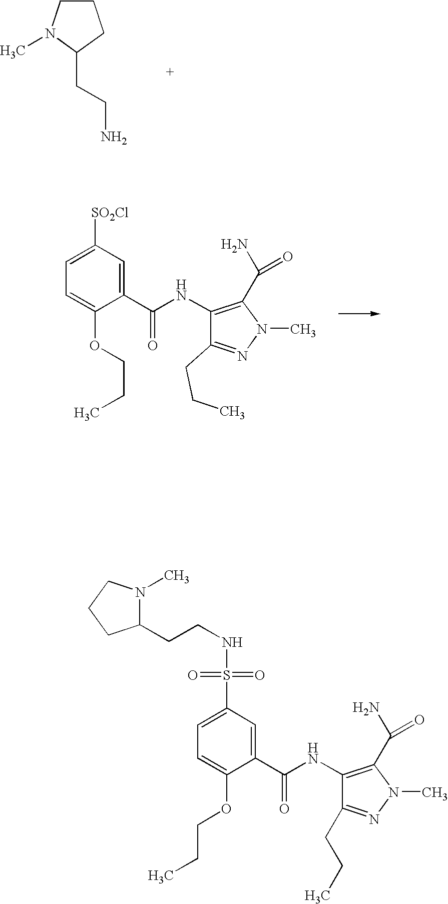

2-Methyl-4-{5-[2-(1-methyl-pyrrolidin-2-yl)-ethylsulfamoyl]-2-propoxy-benzoylamino}-5-propyl-2H-pyrazole-3-carboxamide: To a solution of 3-(5-carbamoyl-1-methyl-3-propyl-1H-pyrazol-4-ylcarbamoyl)-4-propoxy-benzenesulfonyl chloride (2.12 g, 4.8 mmol) and dry triethylamine (0.5 g, 4.8 mmol) in dichloromethane (20 mL), was added 2-(2-aminoethyl)-1-methylpyrrolidine (0.6 g, 4.8 mmol) at 0° C. The reaction was warmed to ambient temperature, stirred for 1 hour at ambient temperature, and diluted with dichloromethane (40 mL). Following standard extractive work up, the solvent was evaporated under reduced pressure to yield the title compound (2.2 g) which was used directly in the next step. LC-MS: m/z=535 (M+H)⁺

### Step 11

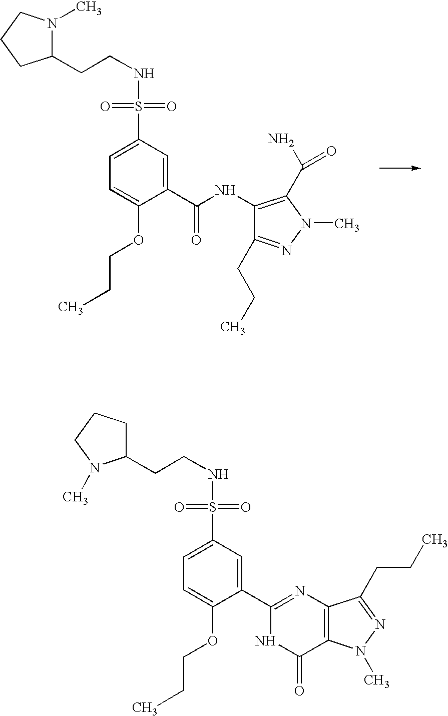

3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide: Potassium tert-butoxide (0.9 g, 8.0 mmol) was added to a solution of crude 2-methyl-4-{5-[2-(1-methyl-pyrrolidin-2-yl)-ethylsulfamoyl]-2-propoxy-benzoylamino}-5-propyl-2H-pyrazole-3-carboxamide (2.14 g, 4.0 mmol) in dry tert-butanol (50 mL), and the mixture was heated to reflux for 8 hours. The reaction mixture was cooled to ambient temperature and diluted with ethyl acetate (300 mL). Following standard extractive work up, the solvent was evaporated under reduced pressure to yield a crude residue which was purified by flash chromatography to give the title compound (1.1 g, 53%). ¹H NMR (300 MHz, CDCl₃) δ 10.90 (broad s, 1H), 8.93 (s, 1H), 7.96 (d, 1H, J=8.7 Hz), 7.15 (d, 1H, J=8.7 Hz), 4.28-4.24 (m, 3H), 4.24 (s, 2H), 3.13 (t, 3H, J=6.9 Hz), 2.93 (t, 3H, J=7.8 Hz), 2.56 (s, 1H), 2.40 (s, 3H), 2.26-2.24 (m, 1H), 2.10-1.99 (m, 2H), 1.89-1.80 (m, 4H), 1.67 (s, 3H, J=7.2 Hz), 1.56-1.52 (m, 1H), 1.22 (t, 3H, J=7.5 Hz), 1.03 (t, 3H, J=7.2 Hz); LC-MS: m/z=517 (MH)⁺

### EXAMPLE 3

### d₃-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide

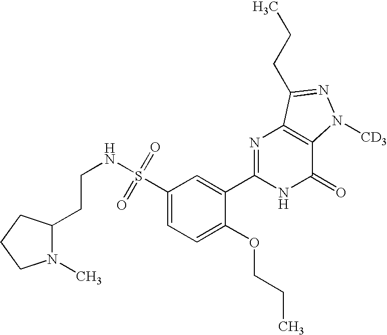

### Step 1

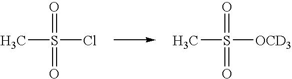

d₃-Methyl methanesulfonate: To a solution of d₄-methanol (16.2 g, 450 mmol) in dry dichloromethane (360 mL) was added dry triethylamine (68.2 g, 675 mmol). The mixture was cooled to −10° C. and methanesulfonyl chloride (77.3 g, 675 mmol) was slowly added while maintaining the temperature below 0° C. The reaction mixture was stirred at 0° C. for 1 hour and washed with water (300 mL). The aqueous layer was extracted with dichloromethane (200 mL). The organic extracts were washed with 3N hydrochloric acid (400 mL), saturated aqueous sodium bicarbonate solution (2×200 mL), dried over anhydrous magnesium sulfate. The solvent was evaporated under reduced pressure to yield product (50 g, 98%), which was used directly in next reaction without further purification. GC-MS: 113 (M)⁺

### Step 2

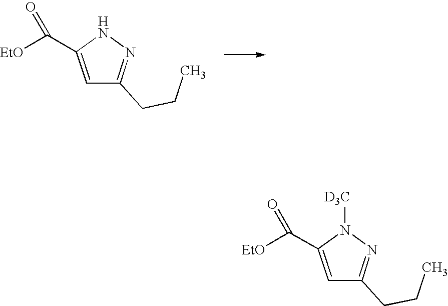

d₃-2-methyl-5-propyl-2H-pyrazole-3-carboxylic acid ethyl ester: The title compound was made by following the procedure set forth in Example 2, Step 3, but by substituting d₃-dimethyl sulfate for dimethyl sulfate. The title compound was a colorless oil (21 g, 59%). LC-MS: m/z=200 (M+H)⁺

### Step 3

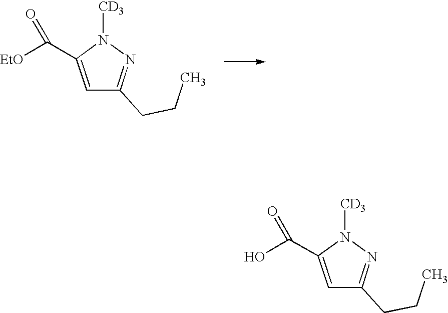

d₃-2-Methyl-5-propyl-2H-pyrazole-3-carboxylic acid: The title compound was made by following the procedure set forth in Example 2, Step 4, but by substituting d₃-2-methyl-5-propyl-2H-pyrazole-3-carboxylic acid ethyl ester for 2-methyl-5-propyl-2H-pyrazole-3-carboxylic acid ethyl ester. The title compound was a white solid (24.2 g, 96%). ¹H NMR (300 MHz, CDCl₃) δ 6.77 (s, 1H), 2.63 (t, 2H, J=7.5 Hz), 1.73-1.66 (m, 2H), 0.99 (t, 3H, J=7.2 Hz); LC-MS: m/z=172 (M+H)⁺

### Step 4

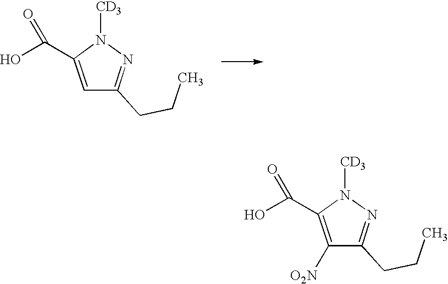

d₃-2-Methyl-4-nitro-5-propyl-2H-pyrazole-3-carboxylic acid: The title compound was made by following the procedure set forth in Example 2, Step 5, but by substituting d₃-2-methyl-5-propyl-2H-pyrazole-3-carboxylic acid for 2-methyl-5-propyl-2H-pyrazole-3-carboxylic acid. The title compound was a white solid (25 g, 92%). LC-MS: m/z=217 (M+H)⁺, 239 (M+Na)⁺

### Step 5

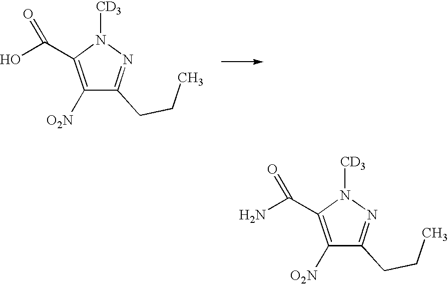

d₃-2-Methyl-4-nitro-5-propyl-2H-pyrazole-3-carboxamide: The title compound was made by following the procedure set forth in Example 2, Step 6, but by substituting d₃-2-methyl-4-nitro-5-propyl-2H-pyrazole-3-carboxylic acid for 2-methyl-4-nitro-5-propyl-2H-pyrazole-3-carboxylic acid. The title compound was an off-white solid (21.5 g, 90%). LC-MS: m/z=216 (M+H)⁺, 238 (M+Na)⁺.

### Step 6

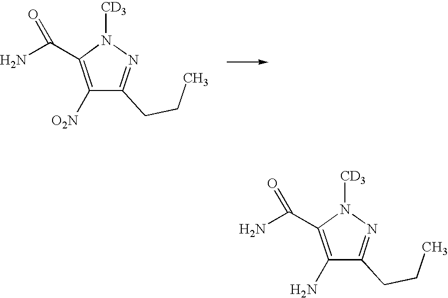

d₃-4-Amino-2-methyl-5-propyl-2H-pyrazole-3-carboxamide: The title compound was made by following the procedure set forth in Example 2, Step 7, but by substituting d₃-2-methyl-4-nitro-5-propyl-2H-pyrazole-3-carboxamide for 2-methyl-4-nitro-5-propyl-2H-pyrazole-3-carboxamide. The title compound (18.7 g, 99%) was used directly in next reaction without further purification.

### Step 7

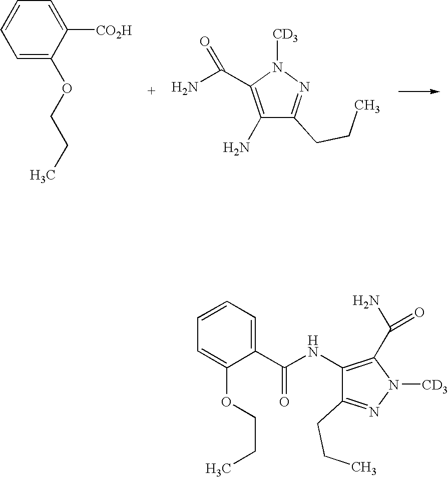

d₃-2-Methyl-4-(2-propoxybenzoylamino)-5-propyl-2H-pyrazole-3-carboxamide: The title compound was made by following the procedure set forth in Example 2, Step 8, but by substituting d₃-4-amino-2-methyl-5-propyl-2H-pyrazole-3-carboxamide for 4-amino-2-methyl-5-propyl-2H-pyrazole-3-carboxamide. The title compound was a white solid (32 g, 91%) that was used directly in next reaction without further purification.

### Step 8

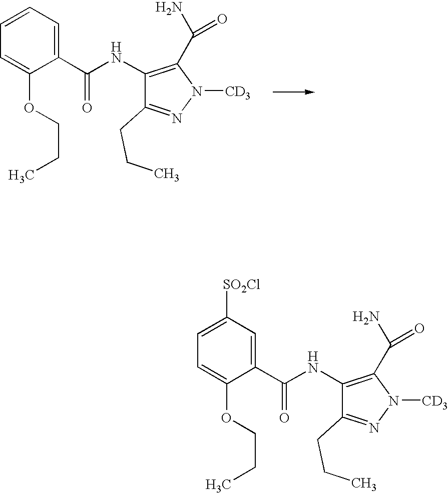

d₃-3-(5-Carbamoyl-1-methyl-3-propyl-1H-pyrazol-4-ylcarbamoyl)-4-propxy-benzenesulfonyl chloride: The title compound was made by following the procedure set forth in Example 2, Step 9, but by substituting d₃-2-Methyl-4-(2-propoxybenzoylamino)-5-propyl-2H-pyrazole-3-carboxamide for 2-Methyl-4-(2-propoxybenzoylamino)-5-propyl-2H-pyrazole-3-carboxamide. The title compound was a white solid (22 g, 54%).

### Step 9

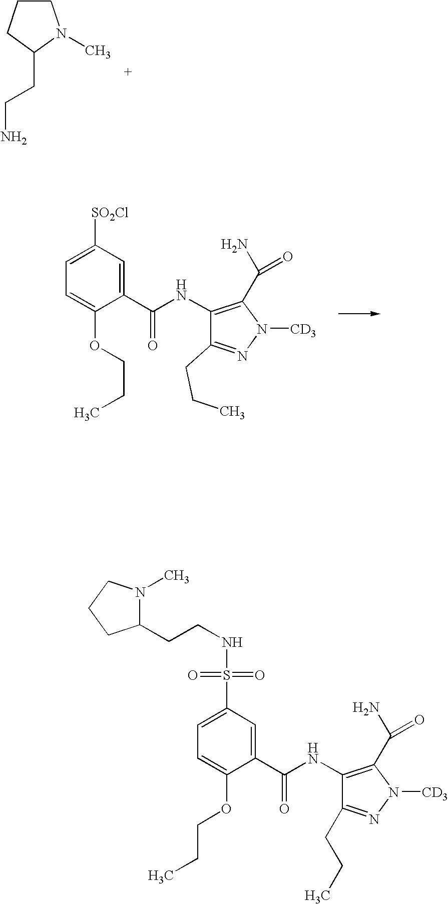

d₃-2-Methyl-4-{5-[2-(1-methyl-pyrrolidin-2-yl)-ethylsulfamoyl]-2-propoxy-benzoylamino}-5-propyl-2H-pyrazole-3-carboxamide: The title compound was made by following the procedure set forth in Example 2, Step 10, but by substituting d₃-3-(5-carbamoyl-1-methyl-3-propyl-1H-pyrazol-4-ylcarbamoyl)-4-propoxy-benzenesulfonyl chloride for 3-(5-carbamoyl-1-methyl-3-propyl-1H-pyrazol-4-ylcarbamoyl)-4-propoxy-benzenesulfonyl chloride. The title compound (2.53 g) was used directly in next reaction without further purification.

### Step 10

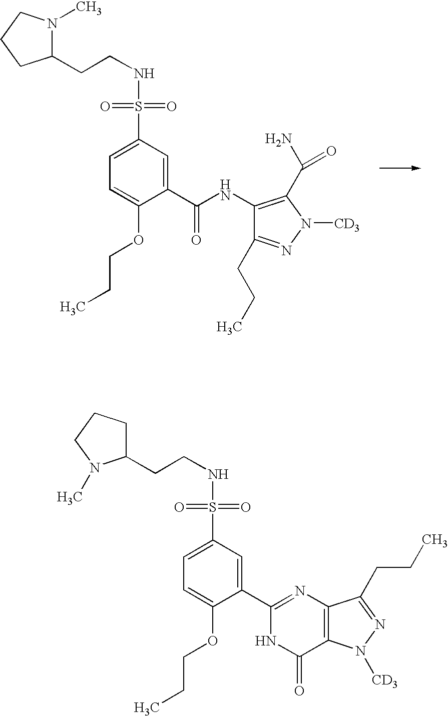

d₃-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide: The title compound was made by following the procedure set forth in Example 2, Step 11, but by substituting d₃-2-methyl-4-{5-[2-(1-methyl-pyrrolidin-2-yl)-ethylsulfamoyl]-2-propoxy-benzoylamino}-5-propyl-2H-pyrazole-3-carboxamide for 2-methyl-4-{5-[2-(1-methyl-pyrrolidin-2-yl)-ethylsulfamoyl]-2-propoxy-benzoylamino}-5-propyl-2H-pyrazole-3-carboxamide. The title compound: (0.9 g, 37%), ¹H NMR (300 MHz, CDCl₃) δ 10.90 (broad s, 1H), 8.94 (s, 1H), 7.96 (d, 1H, J=6.6 Hz), 7.15 (d, 1H, J=8.7 Hz), 4.27 (t, 2H, J=6.0 Hz), 3.16-3.12 (m, 3H), 2.93 (t, 2H, J=7.5 Hz), 2.44 (broad s, 1H), 2.34 (s, 3H), 2.18-2.02 (m, 4H), 1.89-1.74 (m, 4H), 1.63-1.47 (m, 4H), 1.22 (t, 3H, J=7.5 Hz), 1.03 (t, 3H, J=7.2 Hz); LC-MS: m/z=520 (MH)⁺; HPLC: 98% (Purity, 214 nm UV).

### EXAMPLE 4

### d₅-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide

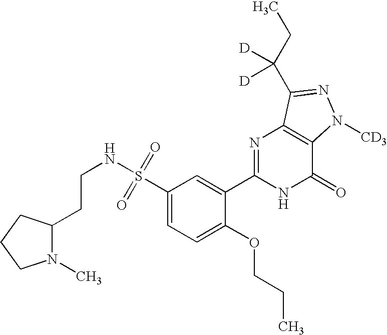

### Step 1

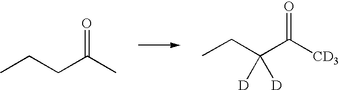

d₅-Pentan-2-one: A suspension containing pentan-2-one (10 g, 116 mmol) and potassium carbonate (1.5 g, 11 mmol) in deuterium oxide (90 mL) was heated at reflux for 72 hours. After cooling, the ketone was salted out of the water layer with excess potassium carbonate. The water layer was discarded and a partially deuterated ketone layer was obtained. This procedure was repeated to give the title compound (9.2 g, 92%) with 98% D-incorporation. ¹H NMR (300 MHz, CDCl₃) δ 1.59 (q, 2H, J=7.2 Hz), 0.91 (t, 3H, J=7.2 Hz).

### Step 2

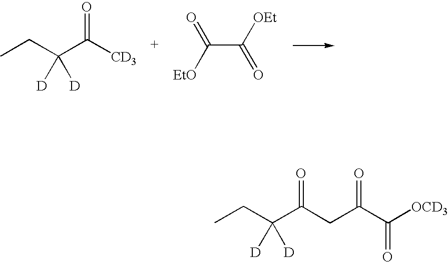

d₅-2,4-dioxo-heptanoic acid methyl ester: Sodium (2.6 g, 110 mmol) was added to d₄-methanol (100 mL) in portions at ambient temperature with vigorous stirring. The solution was cooled to 0° C., d₅-pentan-2-one (9.0 g, 99 mmol) and diethyl oxalate (14.6 g, 100 mmol) were sequentially added at 0° C. The reaction mixture was stirred for another hour at 0° C., warmed to ambient temperature and stirred overnight. The solvent was removed under reduced pressure and following standard acidic extractive work up, the solvent was evaporated under reduced pressure to yield the title compound (11.5 g, 65%). ¹H NMR (300 MHz, CDCl₃) δ 14.51 (br. s, 1H), 6.37 (s, 1H), 1.68 (m, 2H), 0.95 (m, 3H).

### Step 3

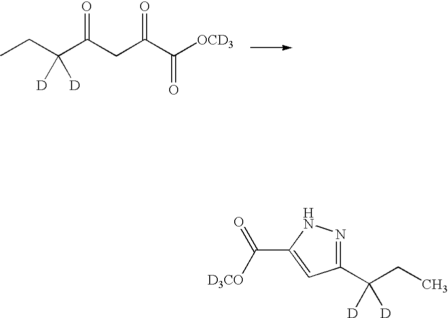

d₅-5-Propyl-2H-pyrazole-3-carboxylic acid methyl ester: The title compound was made by following the procedure set forth in Example 2, Step 2, but by substituting d₅-2,4-dioxo-heptanoic acid methyl ester for 2,4-dioxo-heptanoic acid ethyl ester. The title compound was a white solid (12 g) which was used directly in next reaction without further purification. ¹H NMR (300 MHz, CDCl₃) δ 6.61 (s, 1H), 1.66 (q, 2H, J=7.5 Hz), 0.96 (t, 3H, J=7.5 Hz).

### Step 4

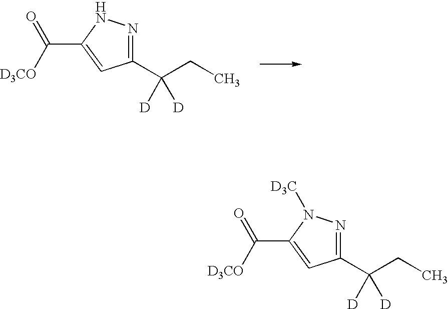

d₈-2-Methyl-5-propyl-2H-pyrazole-3-carboxylic acid methyl ester: The title compound was made by following the procedure set forth in Example 2, Step 3, but by substituting d₅-5-propyl-2H-pyrazole-3-carboxylic acid methyl ester for 5-propyl-2H-pyrazole-3-carboxylic acid ethyl ester. The title compound was a colorless oil (4.4 g, 40%) which was used directly in next reaction without further purification.

### Step 5

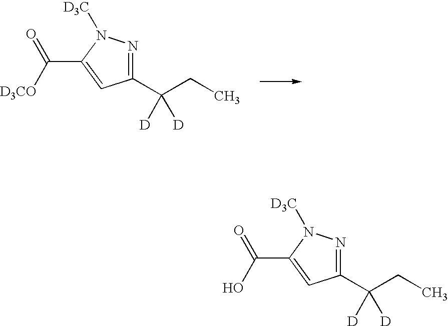

d₅-2-Methyl-5-propyl-2H-pyrazole-3-carboxylic acid: The title compound was made by following the procedure set forth in Example 2, Step 4, but by substituting d₈-2-methyl-5-propyl-2H-pyrazole-3-carboxylic acid methyl ester for 2-methyl-5-propyl-2H-pyrazole-3-carboxylic acid ethyl ester. The title compound was a white solid (3.9 g, 99%). ¹H NMR (300 MHz, CDCl₃) δ 6.76 (s, 1H), 1.67 (q, 2H, J=7.5 Hz), 0.98 (t, 3H, J=7.5 Hz).

### Step 6

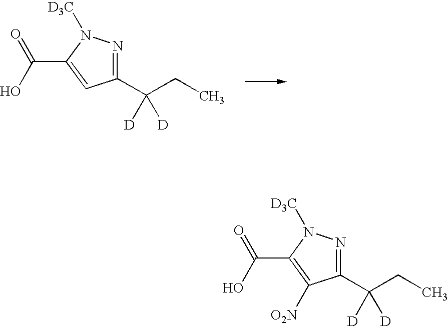

d₅-2-Methyl-4-nitro-5-propyl-2H-pyrazole-3-carboxylic acid: The title compound was made by following the procedure set forth in Example 2, Step 5, but by substituting d₅-2-methyl-5-propyl-2H-pyrazole-3-carboxylic acid for 2-methyl-5-propyl-2H-pyrazole-3-carboxylic acid. The title compound was a white solid (4.8 g, 98%). ¹H NMR (300 MHz, CDCl₃) δ1.72 (q, 2H, J=7.2 Hz), 1.02 (t, 3H, J=7.2 Hz).

### Step 7

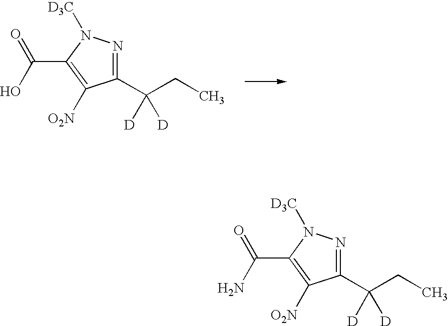

d₅-2-Methyl-4-nitro-5-propyl-2H-pyrazole-3-carboxamide: The title compound was made by following the procedure set forth in Example 2, Step 6, but by substituting d₅-2-methyl-4-nitro-5-propyl-2H-pyrazole-3-carboxylic acid for 2-methyl-4-nitro-5-propyl-2H-pyrazole-3-carboxylic acid. The title compound was an off-white solid (4.7 g, 98%). ¹H NMR (300 MHz, CDCl₃) δ 7.57 (br. s, 1H), 6.10 (br. s, 1H), 1.72 (q, 2H, J=6.9 Hz), 1.02 (t, 3H, J=7.2 Hz).

### Step 8

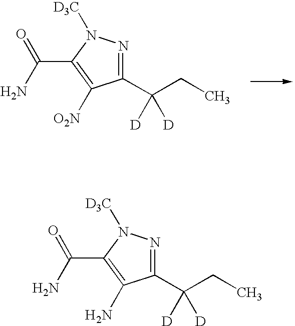

d₅-4-Amino-2-methyl-5-propyl-2H-pyrazole-3-carboxamide: The title compound was made by following the procedure set forth in Example 2, Step 7, but by substituting d₅-2-methyl-4-nitro-5-propyl-2H-pyrazole-3-carboxamide for 2-methyl-4-nitro-5-propyl-2H-pyrazole-3-carboxamide. The title compound (3.7 g, 95%) was used directly in next reaction without further purification.

### Step 9

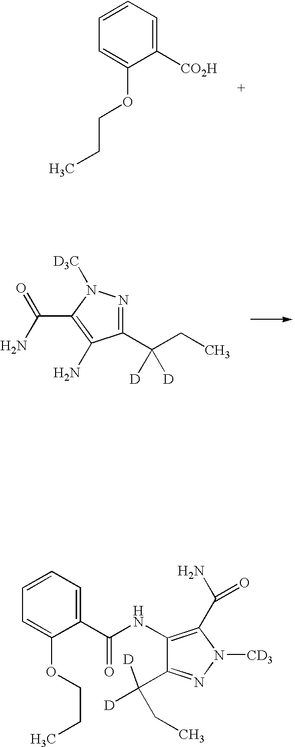

d₅-2-Methyl-4-(2-propoxybenzoylamino)-5-propyl-2H-pyrazole-3-carboxamide: The title compound was made by following the procedure set forth in Example 2, Step 8, but by substituting d₅-4-amino-2-methyl-5-propyl-2H-pyrazole-3-carboxamide for 4-amino-2-methyl-5-propyl-2H-pyrazole-3-carboxamide. The title compound was a white solid (5.1 g, 71%). ¹H NMR (300 MHz, CDCl₃) δ 9.47 (s, 1H), 8.29 (d, 1H, J=7.8 Hz), 7.86 (br. s, 1H), 7.54 (t, 3H, J=7.8 Hz), 7.27-7.06 (m, 2H), 5.65 (br. s, 1H), 4.21 (t, 3H, J=6.6 Hz), 1.94 (q, 2H, J=7.2 Hz), 1.64 (q, 2H, J=7.2 Hz), 1.08 (t, 3H, J=7.2 Hz), 0.93 (t, 3H, J=7.2 Hz).

### Step 10

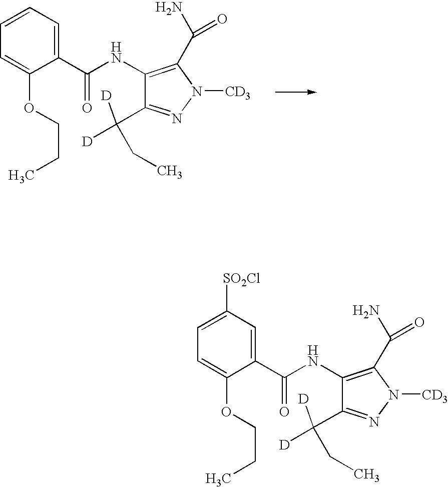

d₅-3-(5-Carbamoyl-1-methyl-3-propyl-1H-pyrazol-4-ylcarbamoyl)-4-propxy-benzenesulfonyl chloride: The title compound was made by following the procedure set forth in Example 2, Step 9, but by substituting d₅-2-methyl-4-(2-propoxybenzoylamino)-5-propyl-2H-pyrazole-3-carboxamide for 2-methyl-4-(2-propoxybenzoylamino)-5-propyl-2H-pyrazole-3-carboxamide. The title compound was a white solid (3.5 g, 53%). ¹H NMR (300 MHz, CDCl₃) δ 9.21 (s, 1H), 8.96 (s, 1H), 8.20 (d, 1H, J=11.4 Hz), 7.61 (br. s, 1H), 7.29-7.26 (m, 2H), 5.65 (br. s, 1H), 4.39-4.34 (m, 2H), 2.21 (br. s, 1H), 2.01 (q, 2H, J=7.2 Hz), 1.65 (q, 2H, J=6.6 Hz), 1.12 (t, 3H, J=7.2 Hz), 0.95 (t, 3H, J=7.2 Hz).

### Step 11

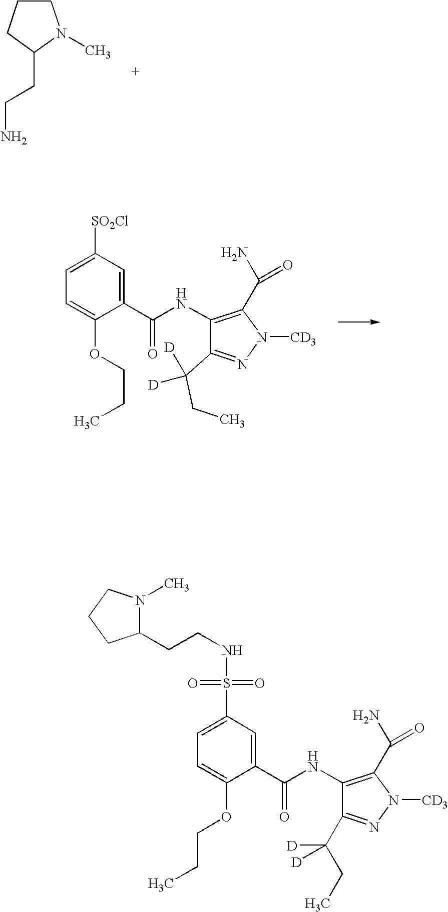

d₅-2-Methyl-4-{5-[2-(1-methyl-pyrrolidin-2-yl)-ethylsulfamoyl]-2-propoxy-benzoylamino}-5-propyl-2H-pyrazole-3-carboxamide: The title compound was made by following the procedure set forth in Example 2, Step 10, but by substituting d₅-3-(5-carbamoyl-1-methyl-3-propyl-1H-pyrazol-4-ylcarbamoyl)-4-propxy-benzenesulfonyl chloride for 3-(5-carbamoyl-1-methyl-3-propyl-1H-pyrazol-4-ylcarbamoyl)-4-propxy-benzenesulfonyl chloride. The title compound was a white solid (2.1 g, 87%). ¹H NMR (300 MHz, CDCl₃): δ 9.29 (s, 1H), 8.70 (s, 1H), 8.00 (d, 1H, J=2.4 Hz), 7.60 (br. s, 1H), 7.16 (d, 1H, J=9.0 Hz), 5.89 (br. s, 1H), 4.27 (t, 2H, J=6.6 Hz), 2.98-2.90 (m, 4H), 2.93 (br. s, 1H), 2.38-1.60 (m, 10H), 1.29-1.24 (m, 2H), 1.09 (t, 3H, J=7.5 Hz), 0.93 (t, 3H, J=7.2 Hz).

### Step 12

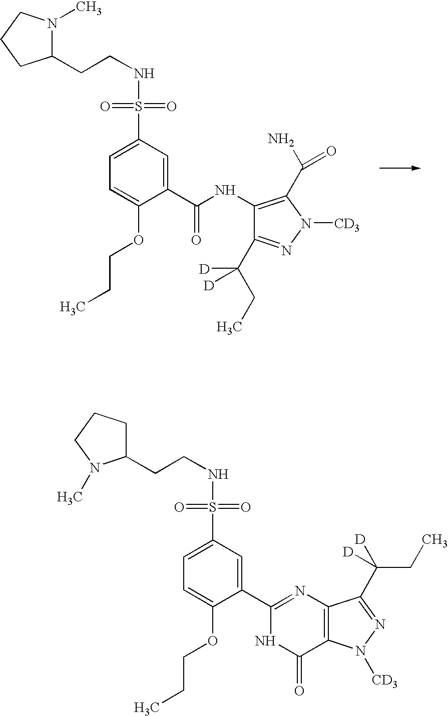

d₅-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide: The title compound was made by following the procedure set forth in Example 2, Step 11, but by substituting d₅-2-methyl-4-{5-[2-(1-methyl-pyrrolidin-2-yl)-ethylsulfamoyl]-2-propoxy-benzoylamino}-5-propyl-2H-pyrazole-3-carboxamide for 2-methyl-4-{5-[2-(1-methyl-pyrrolidin-2-yl)-ethylsulfamoyl]-2-propoxy-benzoylamino}-5-propyl-2H-pyrazole-3-carboxamide. The title compound was a white solid (670 mg, 30%). ¹H NMR (300 MHz, CDCl₃): 10.90 (broad s, 1H), 8.92 (s, 1H), 7.96 (d, 1H, J=8.7 Hz), 7.13 (d, 1H, J=9.6 Hz), 4.26 (t, 2H, J=8.1 Hz), 3.15-3.11 (m, 3H), 2.55-2.07 (m, 5H), 2.05-2.00 (m, 2H), 1.87-1.83 (m, 4H), 1.67-1.51 (m, 4H), 1.18 (t, 3H, J=7.2 Hz), 1.03 (t, 3H, J=7.5 Hz); LC-MS: m/z=522 (M+1); HPLC: 98% (Purity, 214 nm UV)

### EXAMPLE 5

### d₂-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide

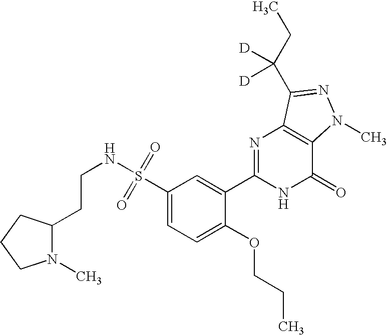

### Step 1

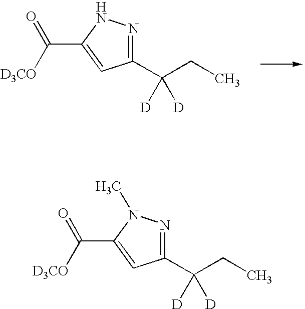

d₅-2-methyl-5-propyl-2H-pyrazole-3-carboxylic acid methyl ester: The title compound was made by following the procedure set forth in Example 2, Step 3, but by substituting d₅-5-propyl-2H-pyrazole-3-carboxylic acid methyl ester for 5-propyl-2H-pyrazole-3-carboxylic acid ethyl ester. The title compound was a colorless oil (5.3 g, 41%).

### Step 2

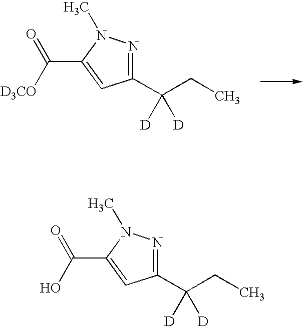

d₂-2-Methyl-5-propyl-2H-pyrazole-3-carboxylic acid: The title compound was made by following the procedure set forth in Example 2, Step 4, but by substituting d₅-2-methyl-5-propyl-2H-pyrazole-3-carboxylic acid methyl ester for 2-methyl-5-propyl-2H-pyrazole-3-carboxylic acid ethyl ester. The title compound was a white solid (4.8 g, 98%). ¹H NMR (300 MHz, CDCl₃) δ 6.76 (s, 1H), 4.16 (s, 3H), 1.67 (q, 2H, J=7.2 Hz), 0.98 (t, 3H, J=7.2 Hz).

### Step 3

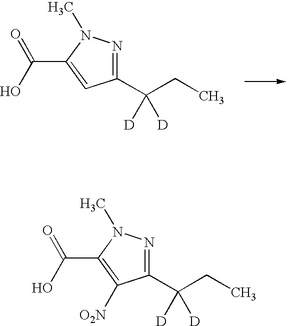

d₂-2-Methyl-4-nitro-5-propyl-2H-pyrazole-3-carboxylic acid: The title compound was made by following the procedure set forth in Example 2, Step 5, but by substituting d₂-2-methyl-5-propyl-2H-pyrazole-3-carboxylic acid for 2-methyl-5-propyl-2H-pyrazole-3-carboxylic acid. The title compound was a white solid (5.9 g, 98%). ¹H NMR (300 MHz, CDCl₃) δ 4.22 (s, 3H), 1.71 (q, 2H, J=7.5 Hz), 1.02 (t, 3H, J=7.5 Hz).

### Step 4

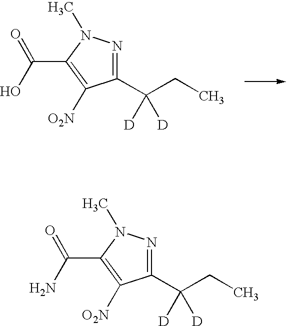

d₂-2-Methyl-4-nitro-5-propyl-2H-pyrazole-3-carboxamide: The title compound was made by following the procedure set forth in Example 2, Step 6, but by substituting d₂-2-methyl-4-nitro-5-propyl-2H-pyrazole-3-carboxylic acid for 2-methyl-4-nitro-5-propyl-2H-pyrazole-3-carboxylic acid. The title compound was an off-white solid (5.8 g, 98%) which was used directly in next reaction without further purification.

### Step 5

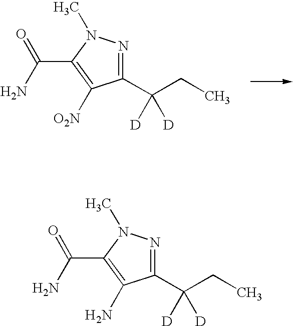

d₂-4-Amino-2-methyl-5-propyl-2H-pyrazole-3-carboxamide: The title compound was made by following the procedure set forth in Example 2, Step 7, but by substituting d₂-2-methyl-4-nitro-5-propyl-2H-pyrazole-3-carboxamide for 2-methyl-4-nitro-5-propyl-2H-pyrazole-3-carboxamide. The title compound (4.2 g, 99%). ¹H NMR (300 MHz, CDCl₃) δ 4.10 (s, 3H), 2.85 (br.s, 2H), 1.65 (q, 2H, J=7.2 Hz), 0.96 (t, 3H, J=7.2 Hz).

### Step 6

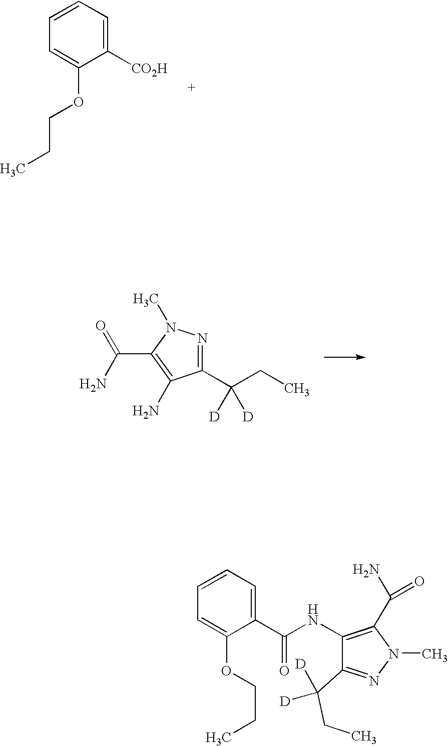

d₂-2-Methyl-4-(2-propoxybenzoylamino)-5-propyl-2H-pyrazole-3-carboxamide: The title compound was made by following the procedure set forth in Example 2, Step 8, but by substituting d₂-4-amino-2-methyl-5-propyl-2H-pyrazole-3-carboxamide for 4-amino-2-methyl-5-propyl-2H-pyrazole-3-carboxamide. The title compound was a white solid (5.6 g, 71%). ¹H NMR (300 MHz, CDCl₃) δ 9.47 (s, 1H), 8.29 (dd, 1H, J=1.8, 8.1 Hz), 7.87 (br. s, 1H), 7.58-7.52 (m, 1H), 7.17-7.06 (m, 2H), 5.64 (br. s, 1H), 4.20 (t, 2H, J=7.2 Hz), 4.08 (s, 3H), 1.97-1.89 (m, 2H), 1.65 (q, 2H, J=7.5 Hz), 1.08 (t, 3H, J=7.5 Hz), 0.94 (t, 3H, J=7.2 Hz).

### Step 7

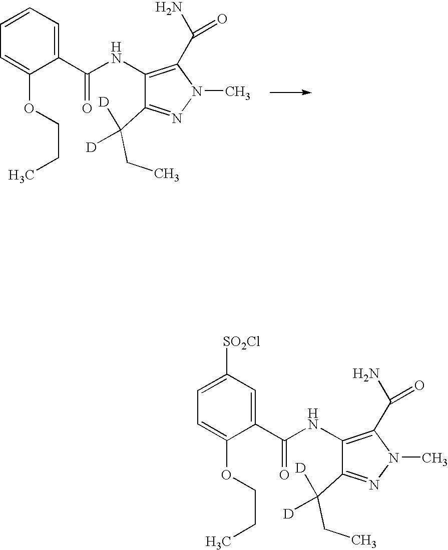

d₂-3-(5-Carbamoyl-1-methyl-3-propyl-1H-pyrazol-4-ylcarbamoyl)-4-propxy-benzenesulfonyl chloride: The title compound was made by following the procedure set forth in Example 2, Step 9, but by substituting d₂-2-methyl-4-(2-propoxybenzoylamino)-5-propyl-2H-pyrazole-3-carboxamide for 2-methyl-4-(2-propoxybenzoylamino)-5-propyl-2H-pyrazole-3-carboxamide. The title compound (3.1 g, 43%). ¹H NMR (300 MHz, DMSO-d₆) δ 9.47 (s, 1H), 7.87 (br. s, 1H), 7.75-7.67 (m, 2H), 7.36 (br. s, 1H), 7.12 (d, 1H, J=7.8 Hz), 4.10 (t, 2H, J=6.6 Hz), 3.91 (s, 3H), 1.80-1.76 (m, 2H), 1.56 (q, 2H, J=7.2 Hz), 0.97 (t, 3H, J=7.5 Hz), 0.88 (t, 3H, J=7.5 Hz).

### Step 8

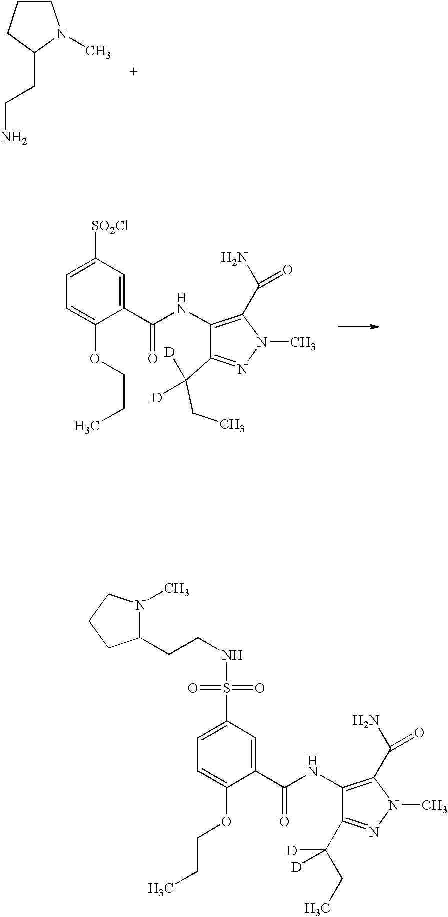

d₂-2-Methyl-4-{5-[2-(1-methyl-pyrrolidin-2-yl)-ethylsulfamoyl]-2-propoxy-benzoylamino}-5-propyl-2H-pyrazole-3-carboxamide: The title compound was made by following the procedure set forth in Example 2, Step 10, but by substituting d₂-3-(5-carbamoyl-1-methyl-3-propyl-1H-pyrazol-4-ylcarbamoyl)-4-propxy-benzenesulfonyl chloride for 3-(5-carbamoyl-1-methyl-3-propyl-1H-pyrazol-4-ylcarbamoyl)-4-propxy-benzenesulfonyl chloride. The title compound: (2.1 g, 87%), ¹H NMR (300 MHz, CDCl₃): δ 9.29 (s, 1H), 8.70 (s, 1H), 8.00 (d, 1H, J=2.4 Hz), 7.60 (br. s, 1H), 7.16 (d, 1H, J=8.7 Hz), 5.82 (br. s, 1H), 4.27 (t, 2H, J=6.6 Hz), 4.25 (s, 3H), 2.98-2.90 (m, 4H), 2.93-2.27 (m, 4H), 1.83-1.47 (m, 9H), 1.47-1.31 (m, 2H), 1.04 (t, 3H, J=7.2 Hz), 0.93 (t, 3H, J=7.2 Hz).

### Step 9

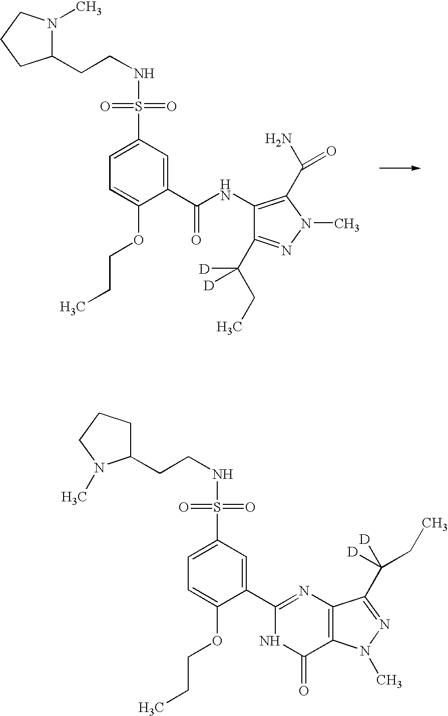

d₂-3-(1-Methyl-7-oxo-3-propyl-6,7-dihydro-1H-pyrazolo[4,3-d]pyrimidin-5-yl)-N-[2-(1-methyl-pyrrolidin-2-yl)-ethyl]-4-propoxy-benzenesulfonamide: The title compound was made by following the procedure set forth in Example 2, Step 11, but by substituting d₂-2-Methyl-4-{5-[2-(1-methyl-pyrrolidin-2-yl)-ethylsulfamoyl]-2-propoxy-benzoylamino}-5-propyl-2H-pyrazole-3-carboxamide for 2-Methyl-4-{5-[2-(1-methyl-pyrrolidin-2-yl)-ethylsulfamoyl]-2-propoxy-benzoylamino}-5-propyl-2H-pyrazole-3-carboxamide. The title compound: (330 mg, 30%), ¹H NMR (300 MHz, CDCl₃): δ 10.90 (br. s, 1H), 8.93 (s, 1H), 7.96 (d, 1H, J=9.3 Hz), 7.15 (d, 1H, J=9.3 Hz), 4.29-4.25 (m, 5H), 3.16-3.13 (m, 3H), 2.46 (br. s, 1H), 2.35 (s, 3H), 2.19-1.99 (m, 3H), 1.88-1.75 (m, 4H), 1.63-1.49 (m, 4H), 1.20 (t, 3H, J=7.5 Hz), 1.01 (t, 3H, J=7.5 Hz); LC-MS: m/z=519 (M+1);

Changes in the metabolic properties of the compounds in Example 1, 3 and 4 as compared to their non-isotopically enriched analogs can be shown using the following assays. Other compounds listed above, which have not yet been made and/or tested, are predicted to have changed metabolic properties as shown by one or more of these assays as well.

### Biological Assays

### EXAMPLE 6

### In Vitro Metabolism Using Human Cytochrome P₄₅₀ Enzymes

The cytochrome P₄₅₀ enzymes are expressed from the corresponding human cDNA using a baculovirus expression system (BD Biosciences). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula 1, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g. acetonitrile, 20% trichloroacetic acid, 94% acetonitrile/6% glacial acetic acid, 70% perchloric acid, 94% acetonitrile/6% glacial acetic acid) and centrifuged (10,000 g) for 3 minutes. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]—(S)-mephenytoin |
| CYP2C8 | Paclitaxel |
| CYP2C9 | Diclofenac |
| CYP2C19 | [¹³C]—(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### EXAMPLE 7

### Monoamine Oxidase A Inhibition and Oxidative Turnover

The procedure is carried out as described in Weyler, Journal of Biological Chemistry 1985, 260(24), 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

### EXAMPLE 8

### 5 Monoamine Oxidase B Inhibition and Oxidative Turnover

The procedure is carried out as described in Uebelhack, Pharmacopsychiatry 1998, 31(5), 187-192, which is hereby incorporated by reference in its entirety.

### EXAMPLE 9

### MAO Assay

Fresh PRP or frozen platelet suspension (100 μl) is generally preincubated for 10 minutes in the absence or presence of drugs at 37° C. in 100 μl of 0.9% NaCl solution or phosphate buffer pH 7.4, respectively, at 37° C. 2-Phenyllethylamine-[ethyl-1-¹⁴C]hydrochloride (PEA) solution (specific activity 56 Ci/mol, Amersham, 50 μl) is then added in a final concentration of 5 μM and the incubation is continued for 30 minutes. The reaction is terminated by the addition of 50 μl 4M HClO₄. The reaction product of MAO, phenylacetaldehyde, is extracted into 2 mL of n-hexane. An aliquot of the organic phase is added to scintillator cocktail and the radioactivity is determined using a liquid scintillation counter. Product formation is linear with time for at least 60 min with appropriate platelet numbers. Blank values are obtained by including 2 mM pargyline in the incubation mixtures.

### EXAMPLE 10

### Determination of Phosphodiesterase Activity

Test substances are assayed against the available phosphodiesterases by the previously described method in Daugan et al, Journal of Medicinal Chemistry 2003, 21(46), 4525-4532, and Daugan et al, Journal of Medicinal Chemistry, 2003, 46, 4533-4542, both of which are hereby incorporated by reference in their entireties.

### EXAMPLE 11

### In Vitro Liver Microsomal Stability Assay

Liver microsomal stability assays were conducted at 1 mg per mL liver microsome protein with an NADPH-generating system in 2% NaHCO₃ (2.2 mM NADPH, 25.6 mM glucose 6-phosphate, 6 units per mL glucose 6-phosphate dehydrogenase and 3.3 mM MgCl₂). Test compounds were prepared as solutions in 20% acetonitrile-water and added to the assay mixture (final assay concentration 5 microgram per mL) and incubated at 37° C. Final concentration of acetonitrile in the assay were <1%. Aliquots (50 μL) were taken out at times 0, 15, 30, 45, and 60 minutes, and diluted with ice cold acetonitrile (200 μL) to stop the reactions. Samples were centrifuged at 12000 RPM for 10 minutes to precipitate proteins. Supernatants were transferred to microcentrifuge tubes and stored for LC/MS/MS analysis of the degradation half-life of the test compounds. It has thus been found that the compounds of formula (I) according to the present invention that have been tested in this assay showed an increase of 10% or more in the degradation half-life, as compared to the non-isotopically enriched drug. For example, the degradation half-life of Examples 4 and 5 were increased by 12-15% as compared to non-isotopically enriched udenafil.

The examples set forth above are disclosed to give a complete disclosure and description of how to make and use the claimed embodiments, and are not intended to limit the scope of what is disclosed herein. Modifications that are obvious, in the art, are intended to be within the scope of the following claims. All publications, patents, and patent applications cited in this specification are incorporated herein by reference as if each such publication, patent or patent application were specifically and individually indicated to be incorporated herein by reference. However, with respect to any similar or identical terms found in both the incorporated publications or references and those explicitly put forth or defined in this document, then those terms definitions or meanings explicitly put forth in this document shall control in all respects.

What is claimed is:

1. A compound having structural Formula I 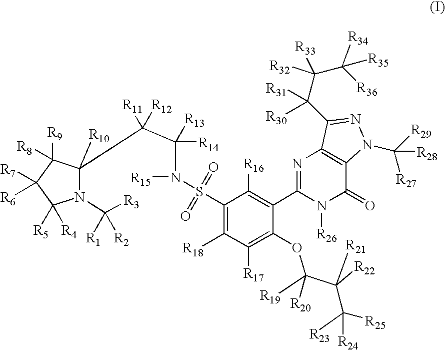
or a pharmaceutically acceptable salt, solvate, or prodrug thereof, wherein:
R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are independently selected from the group consisting of hydrogen, and deuterium; and
at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ is deuterium.

2. The compound as recited in claim 1 wherein said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

3. The compound as recited in claim 1, wherein at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ independently has deuterium enrichment of no less than about 1%.

4. The compound as recited in claim 1, wherein at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ independently has deuterium enrichment of no less than about 5%.

5. The compound as recited in claim 1, wherein at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ independently has deuterium enrichment of no less than about 10%.

6. The compound as recited in claim 1, wherein at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ independently has deuterium enrichment of no less than about 20%.

7. The compound as recited in claim 1, wherein at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ independently has deuterium enrichment of no less than about 50%.

8. The compound as recited in claim 1, wherein at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ independently has deuterium enrichment of no less than about 90%.

9. The compound as recited in claim 1, wherein at least one of R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ independently has deuterium enrichment of no less than about 98%.

10. The compound as recited in claim 1, having a structural formula selected from the group consisting of:

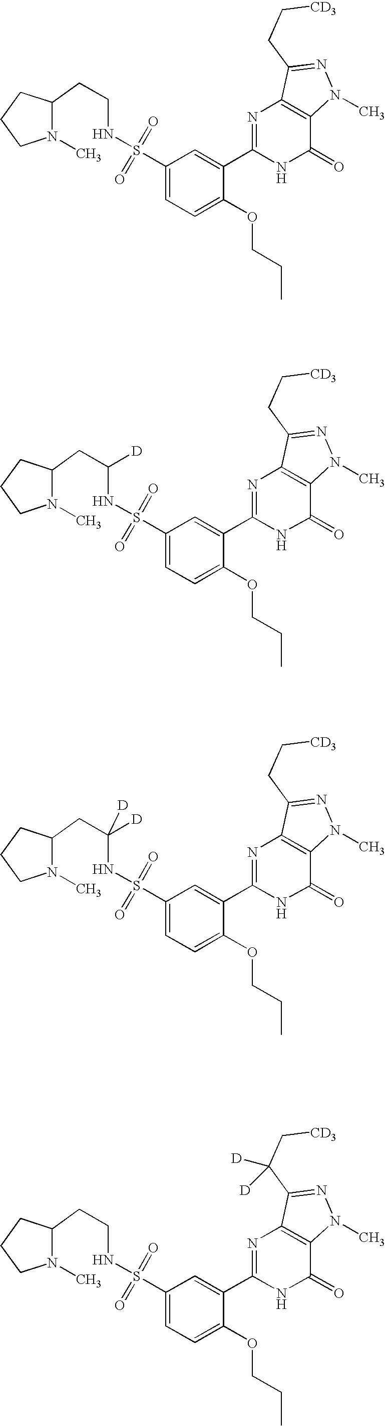

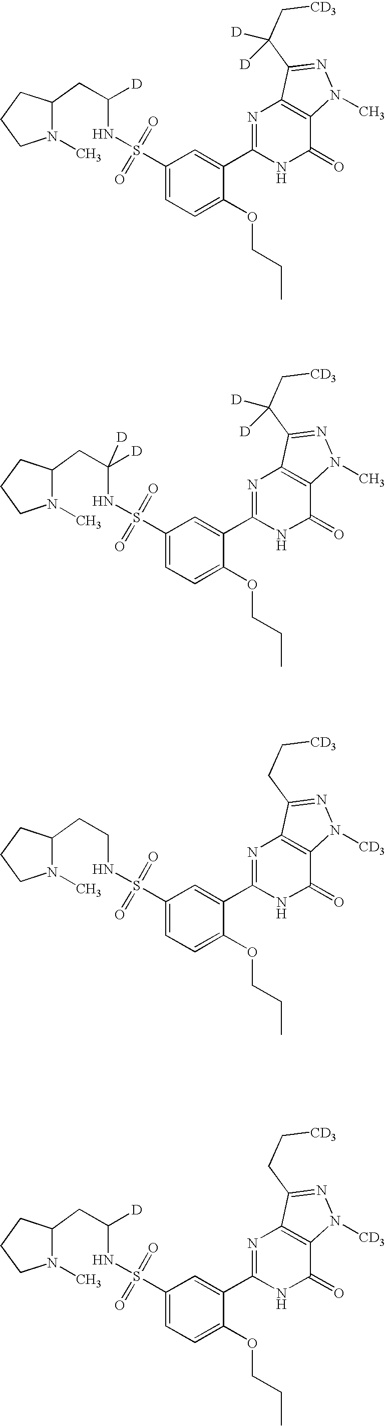

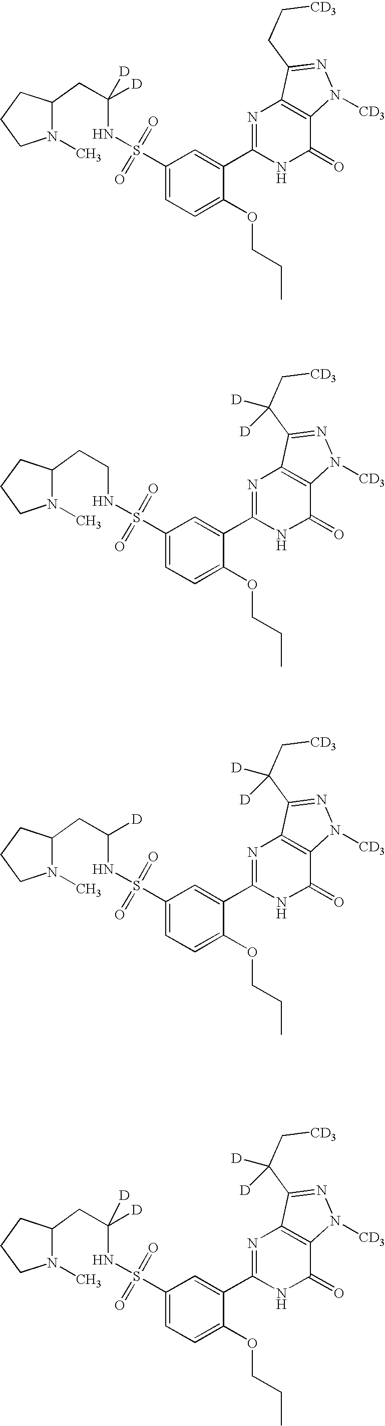

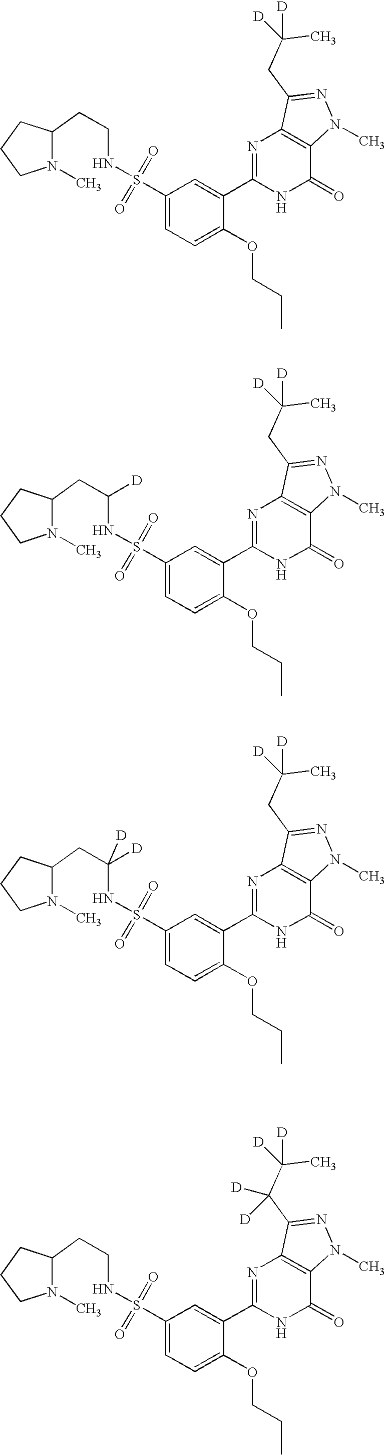

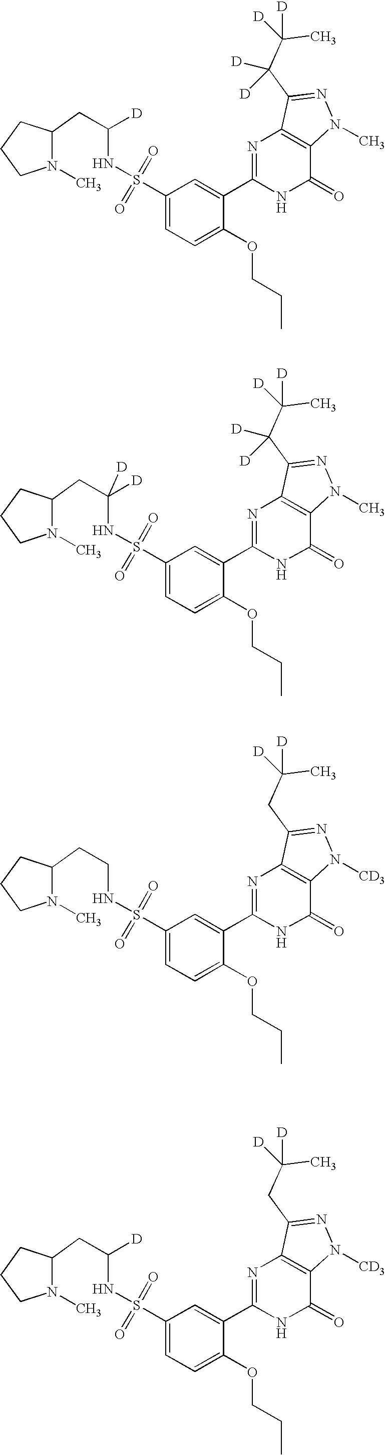

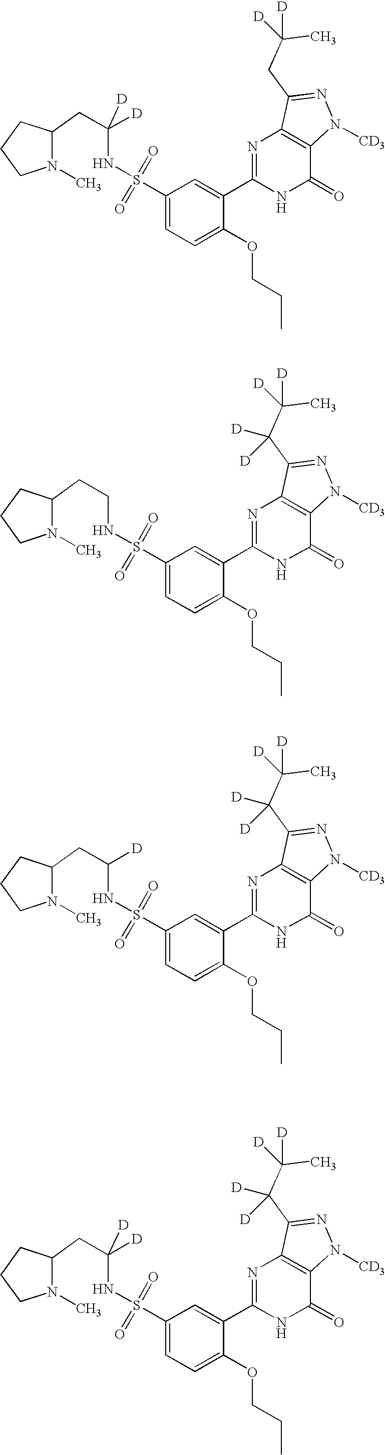

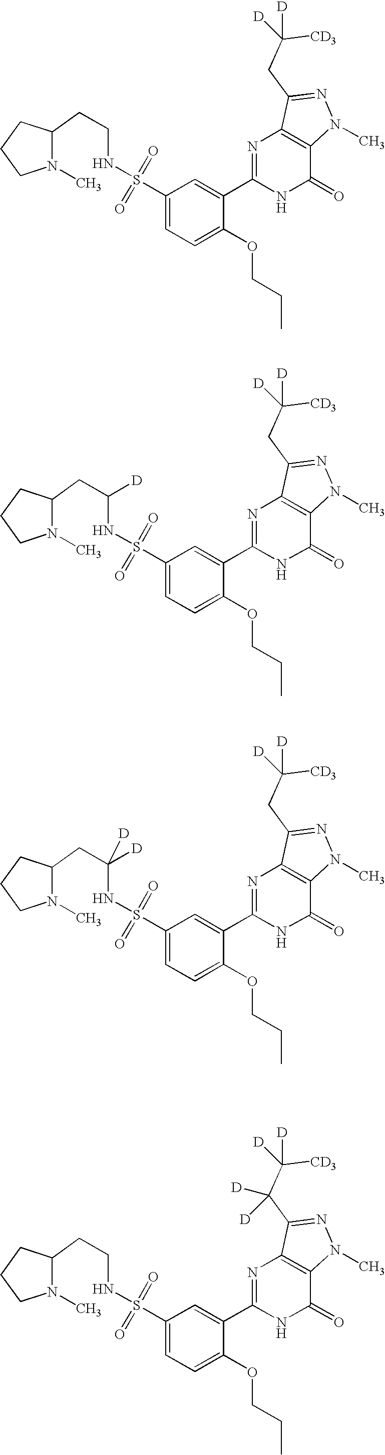

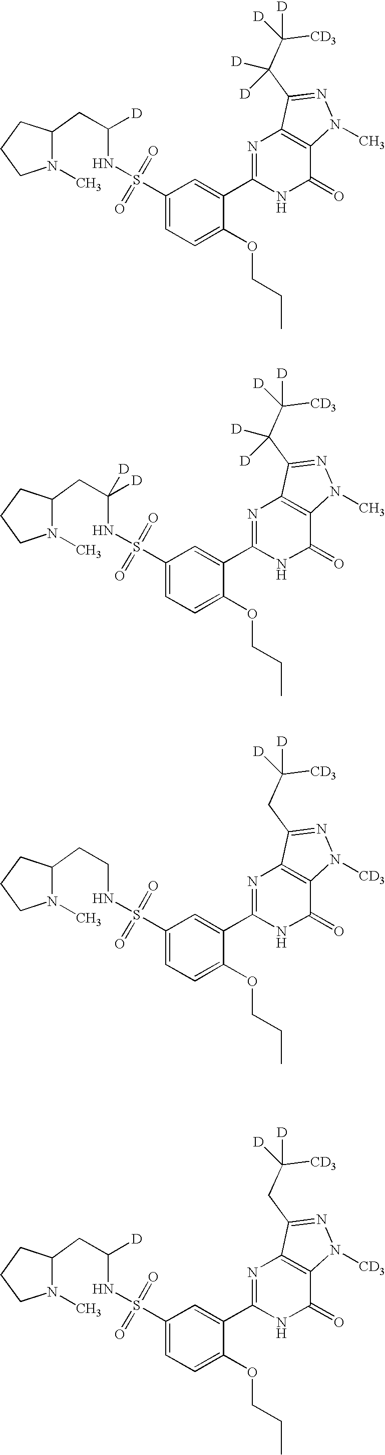

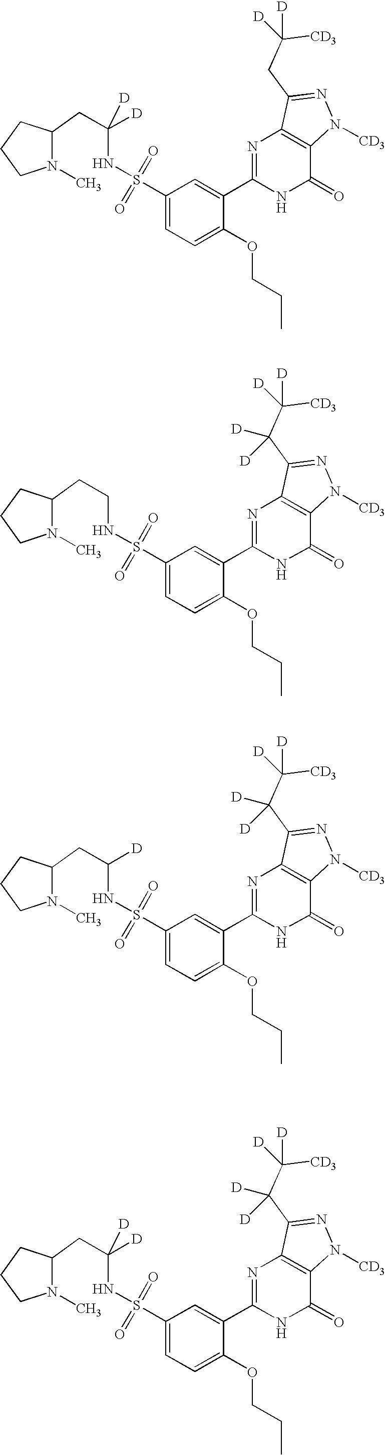

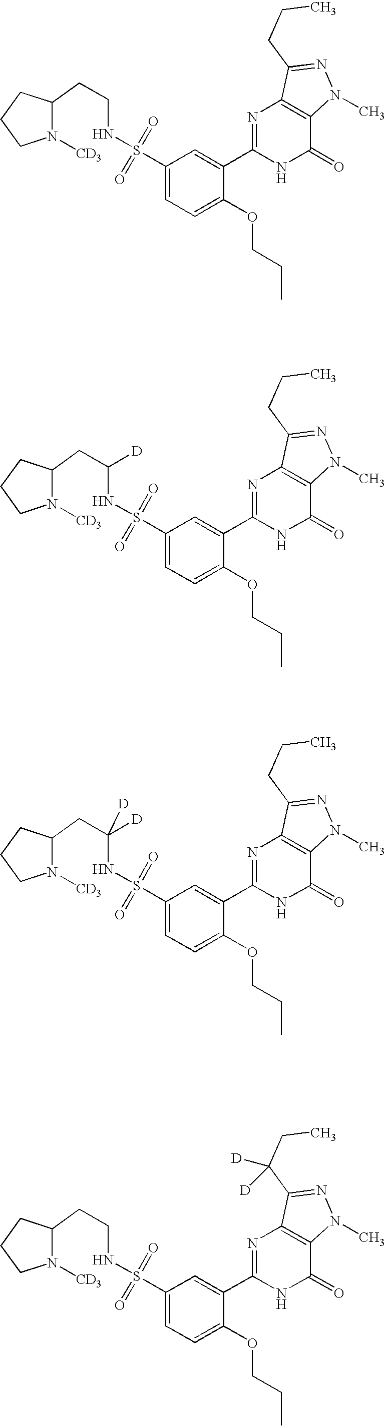

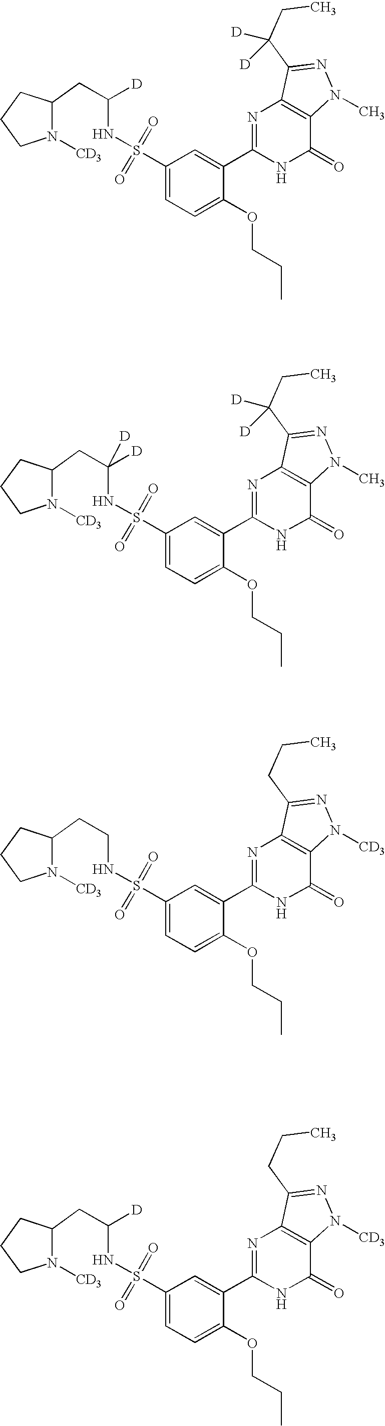

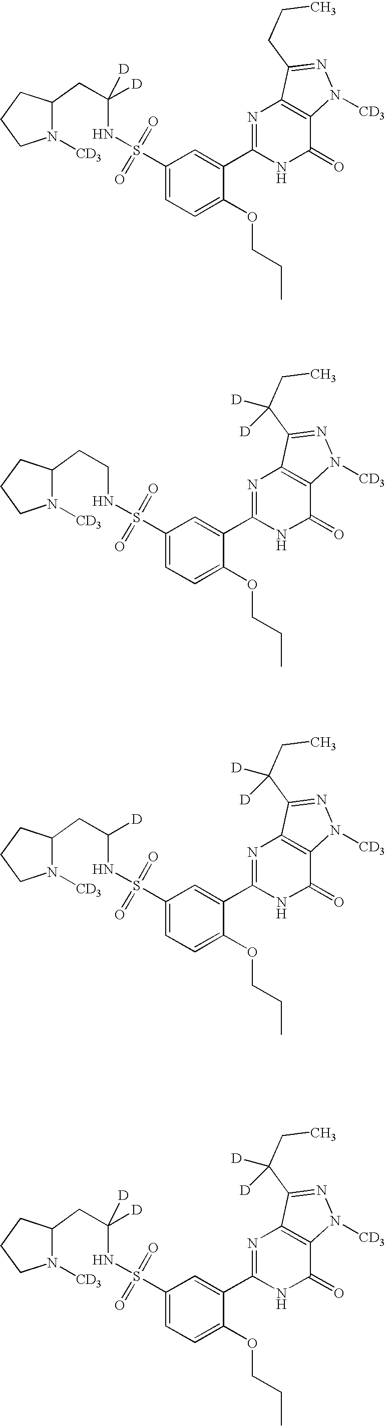

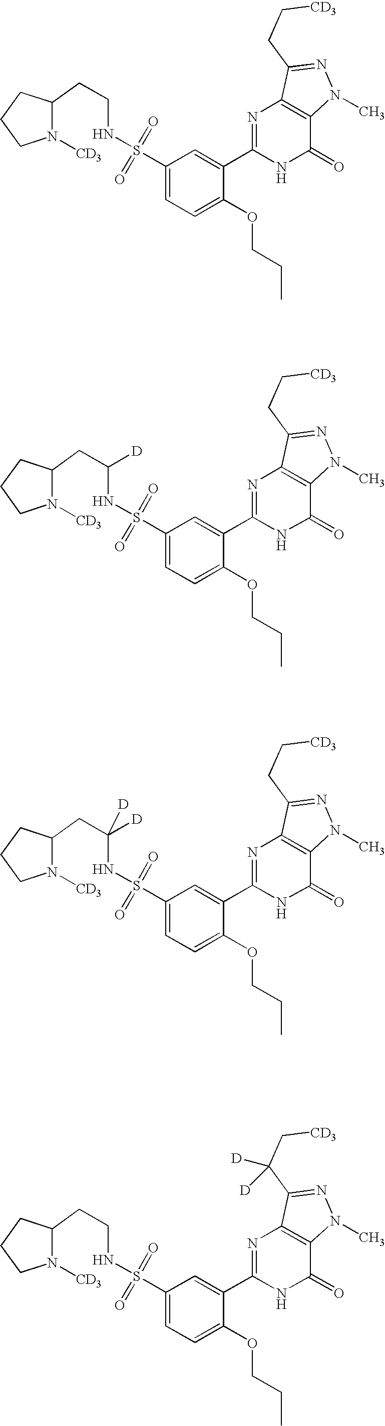

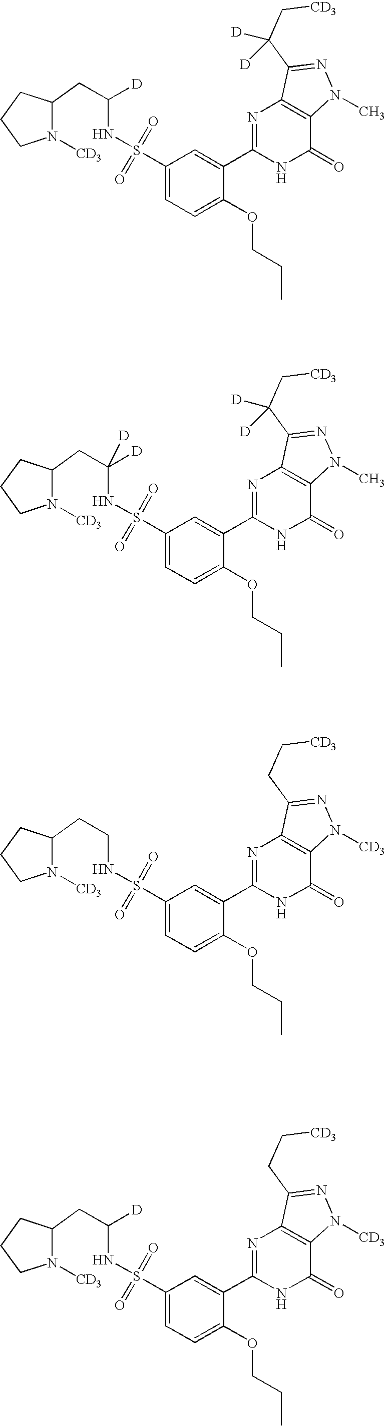

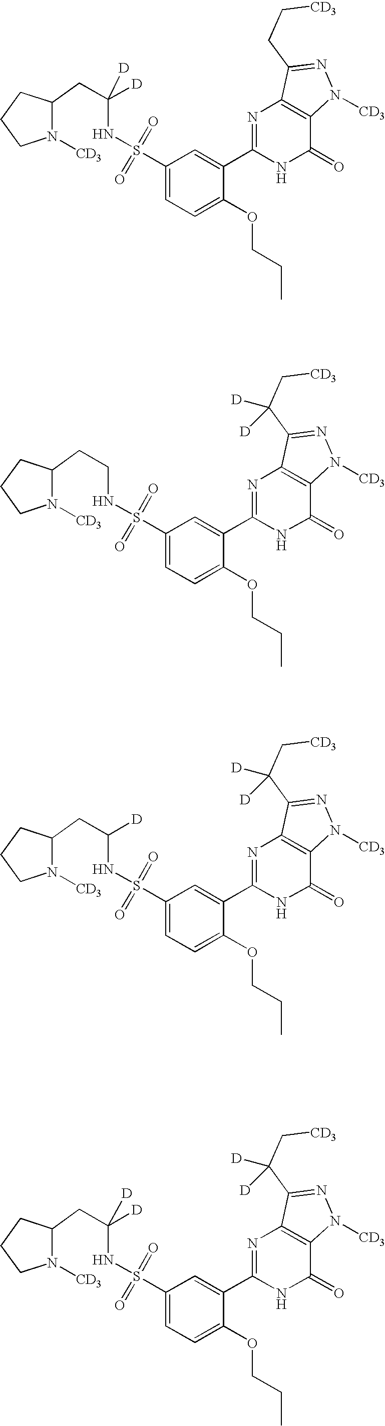

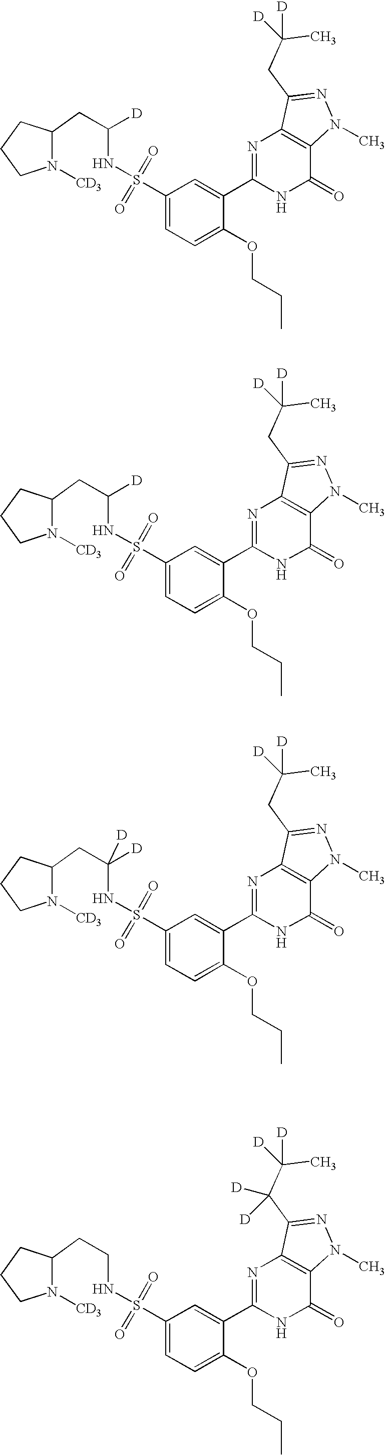

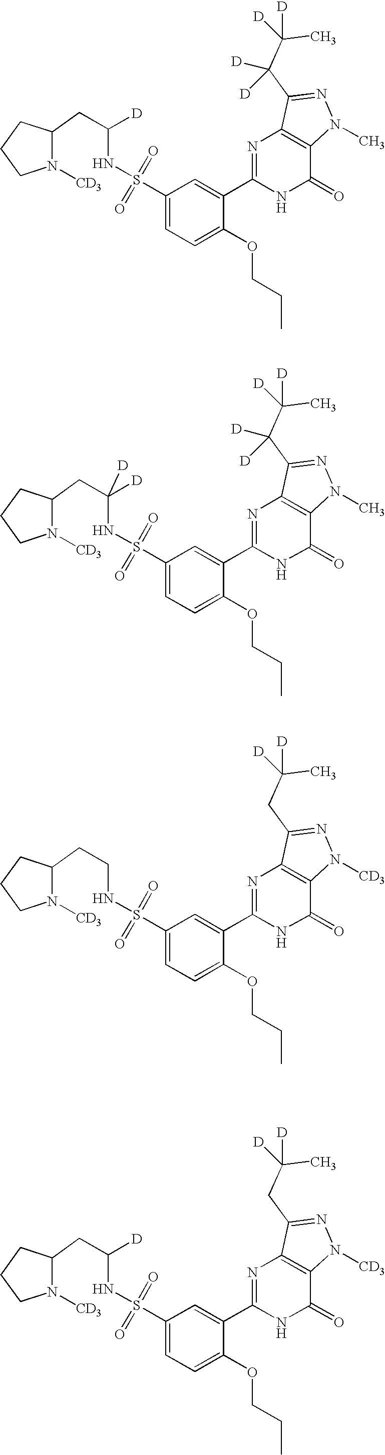

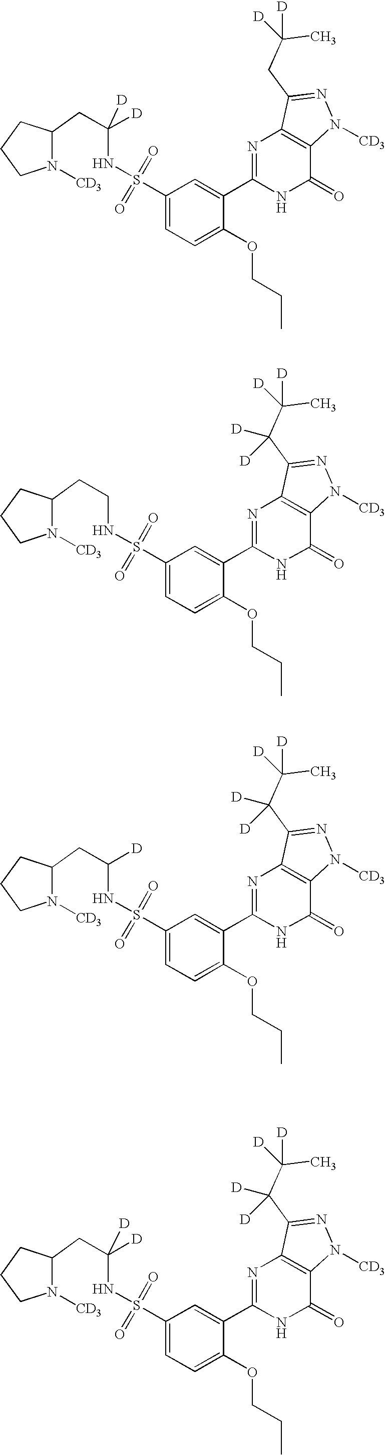

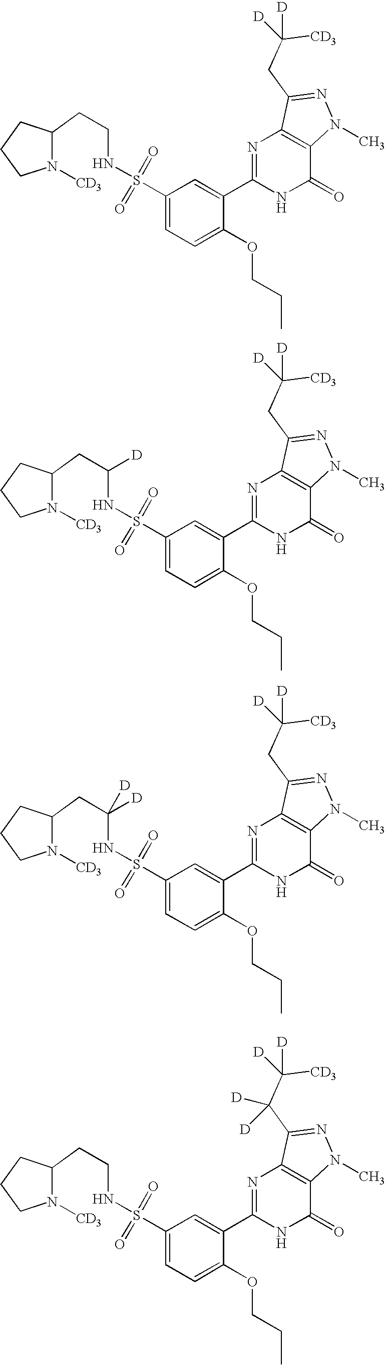

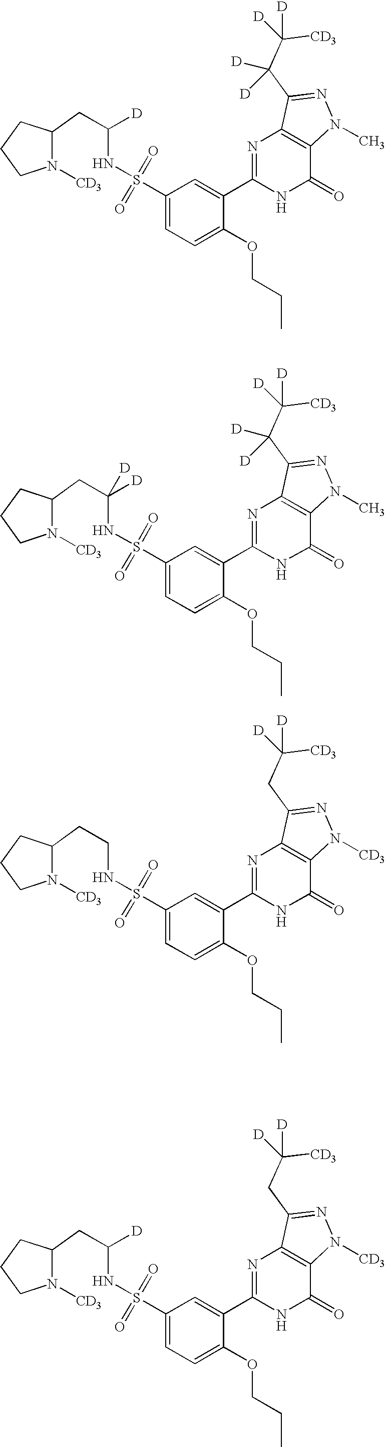

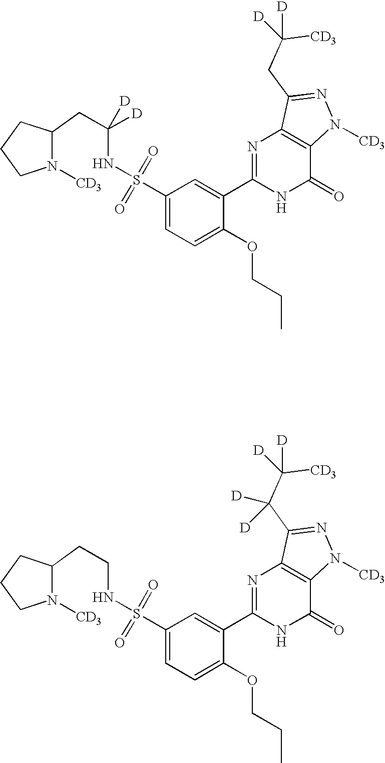

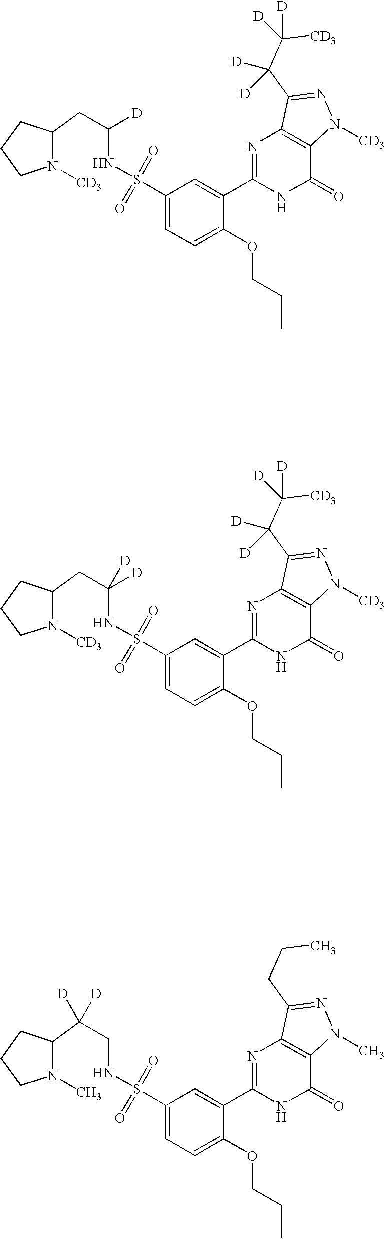

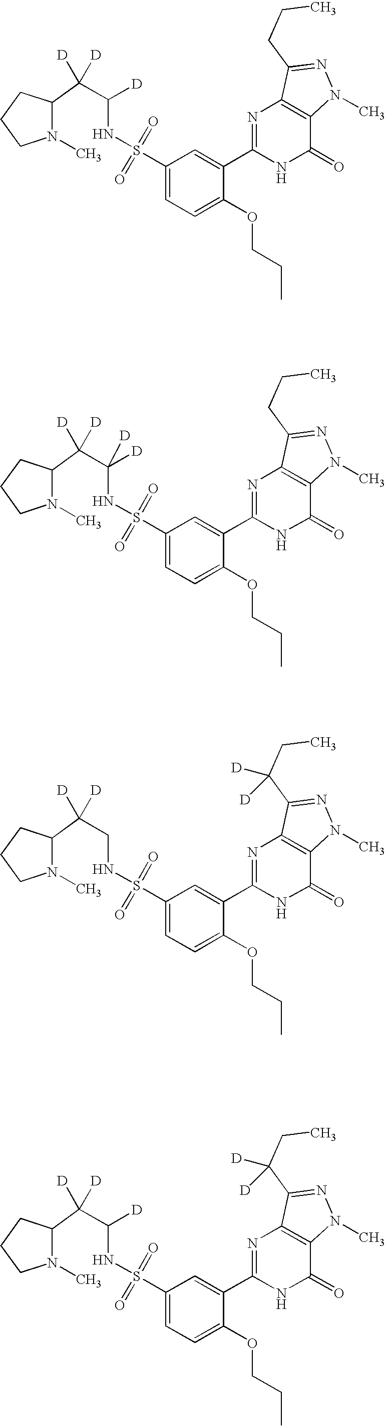

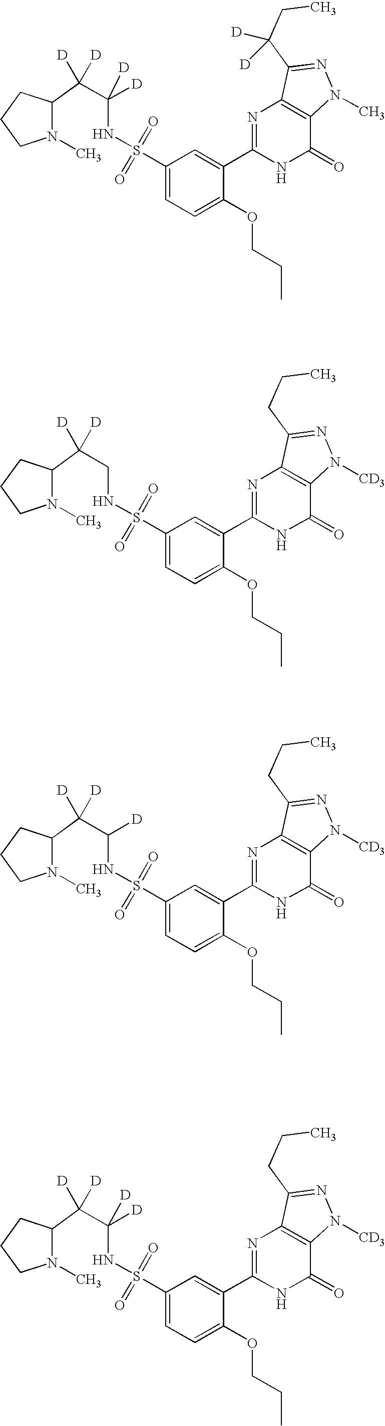

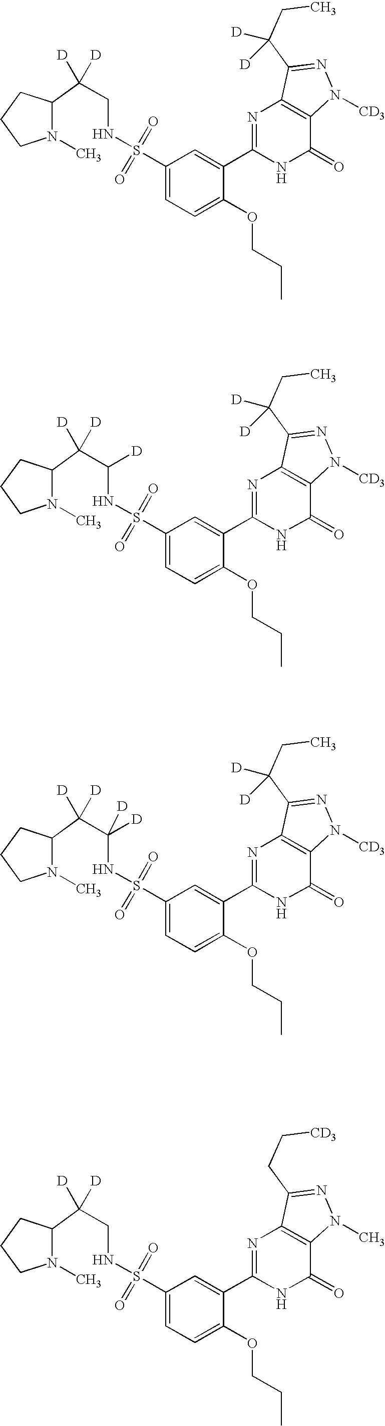

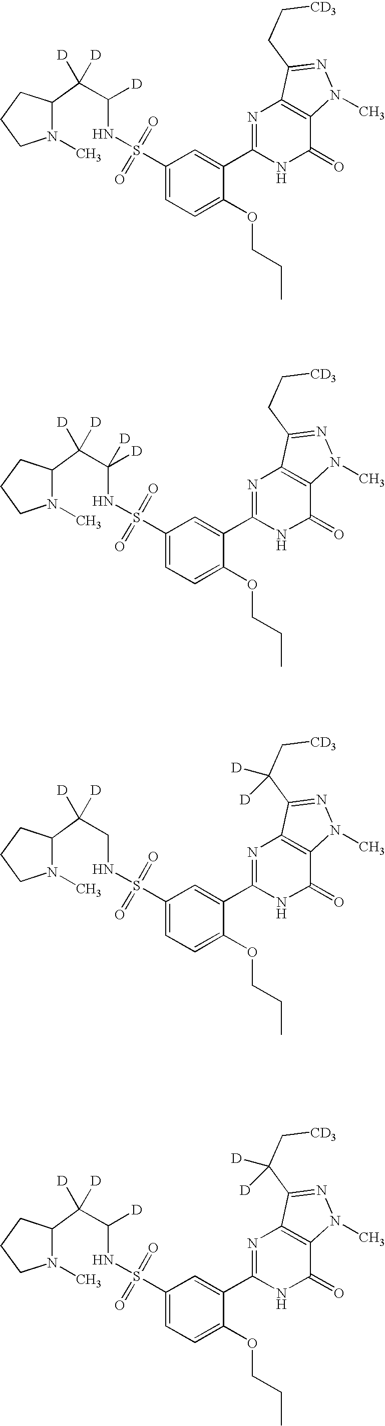

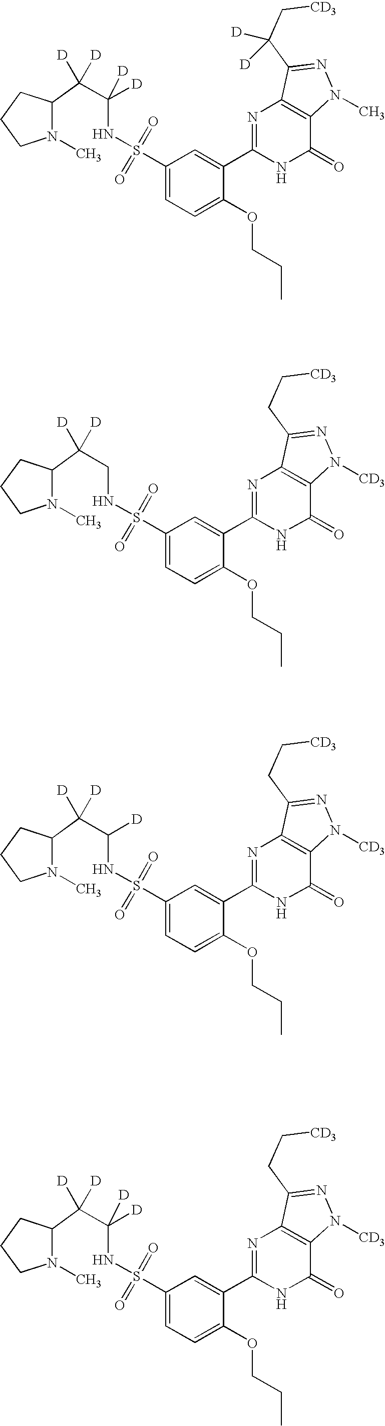

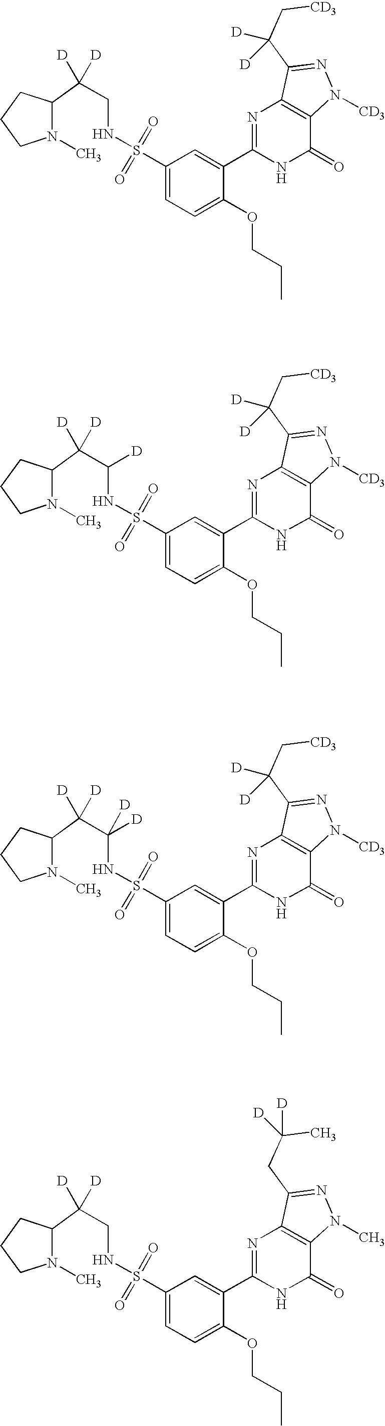

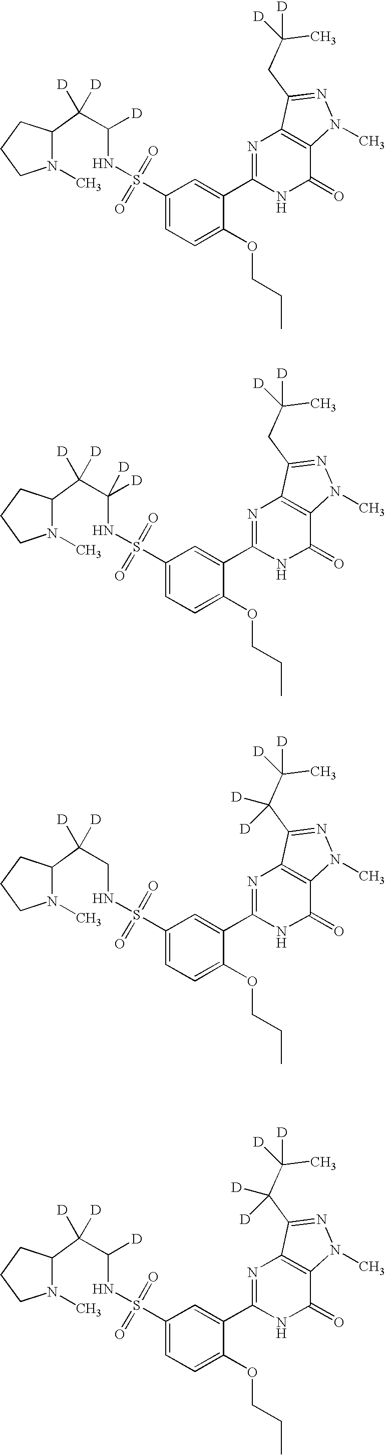

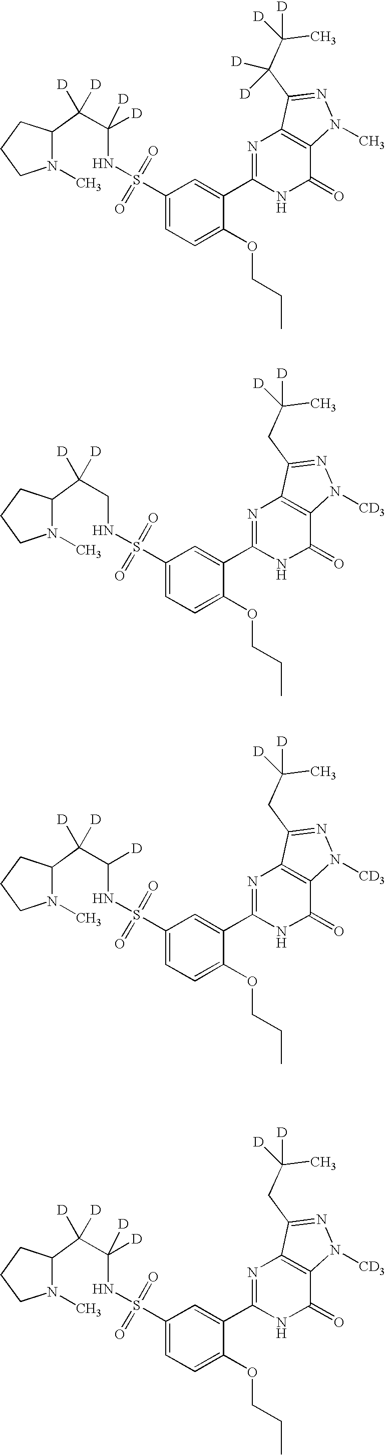

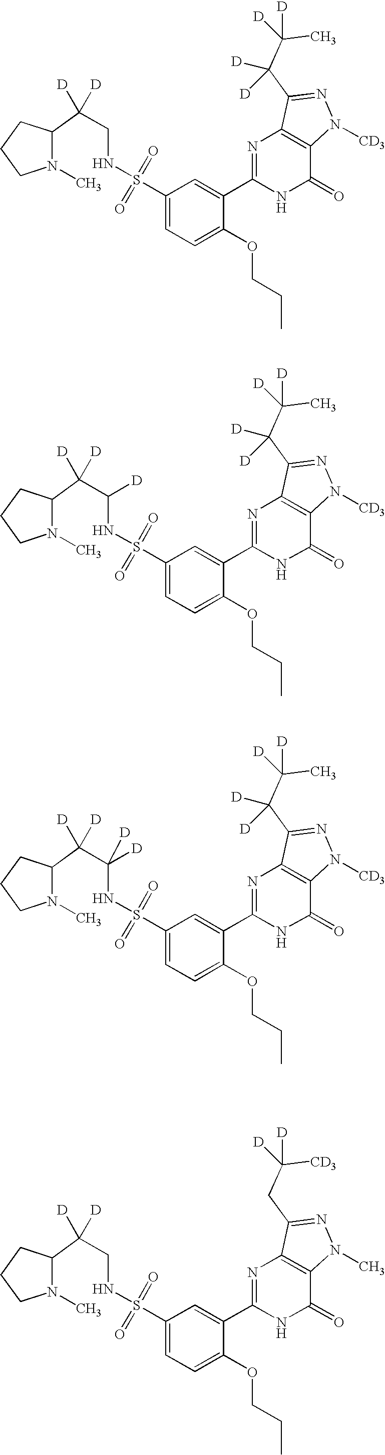

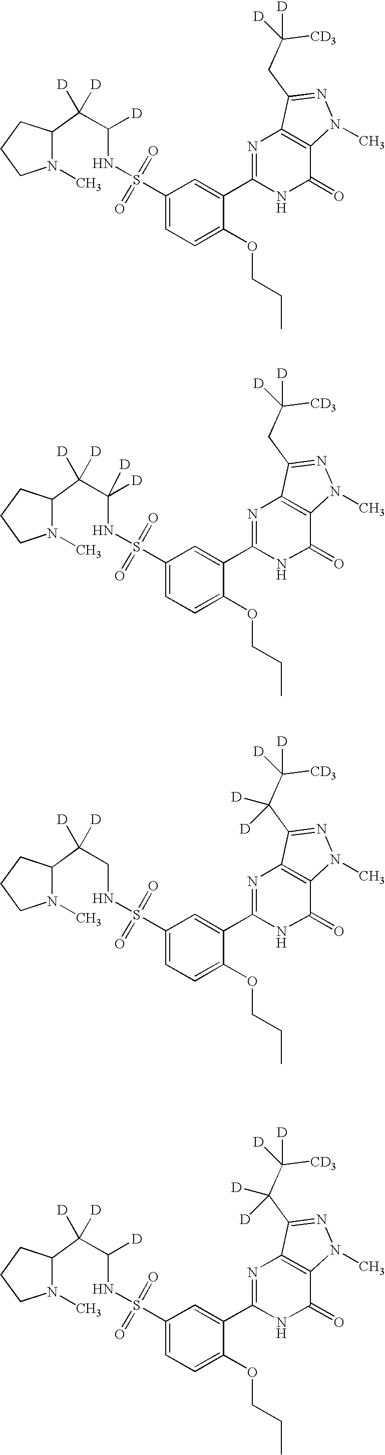

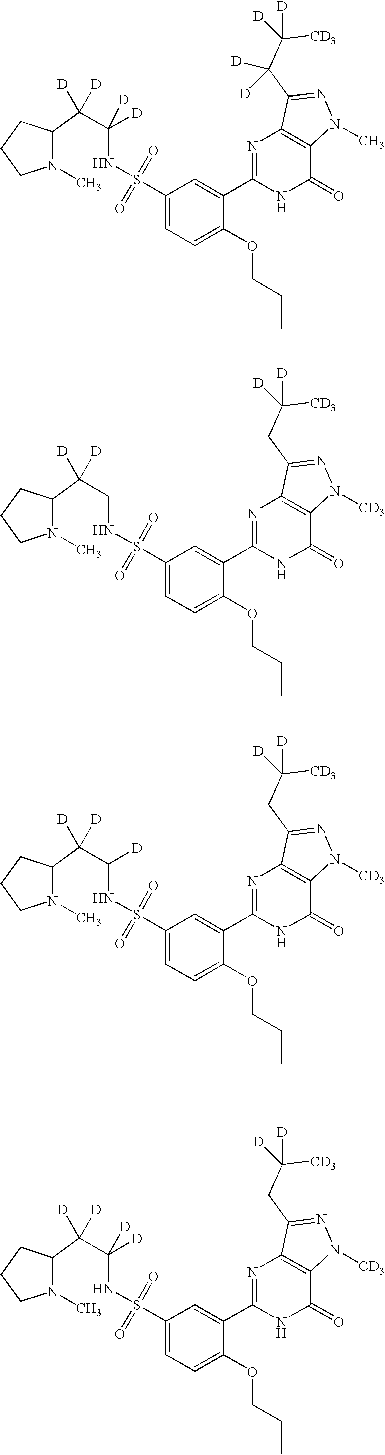

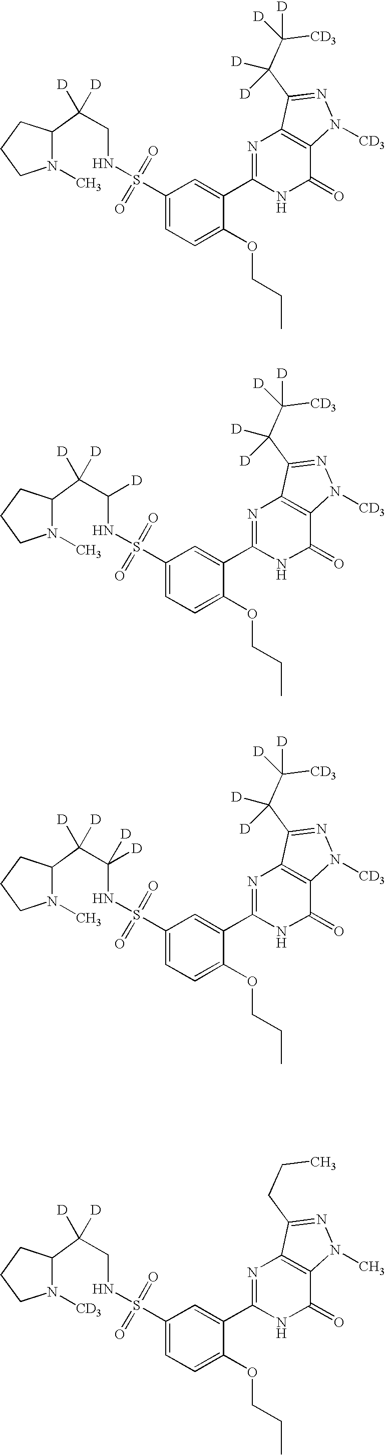

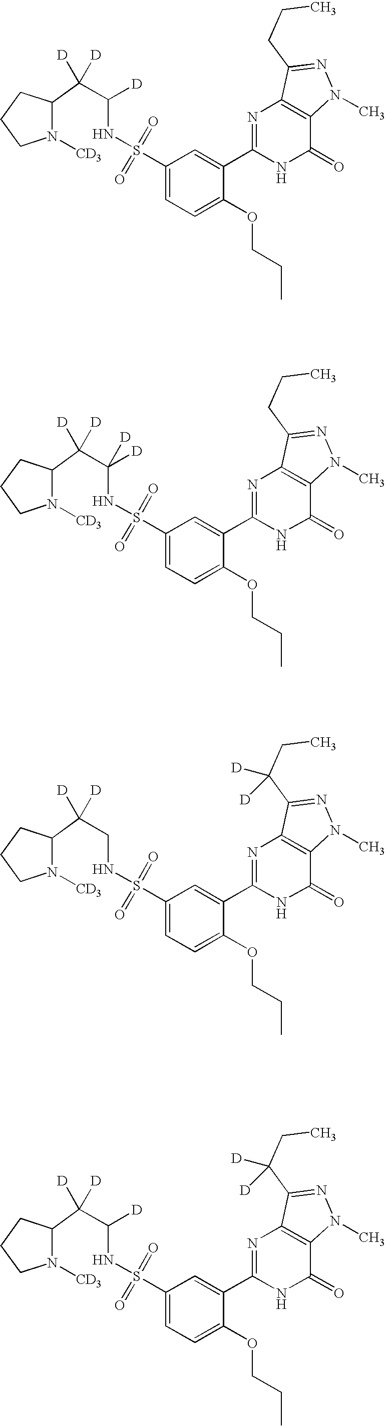

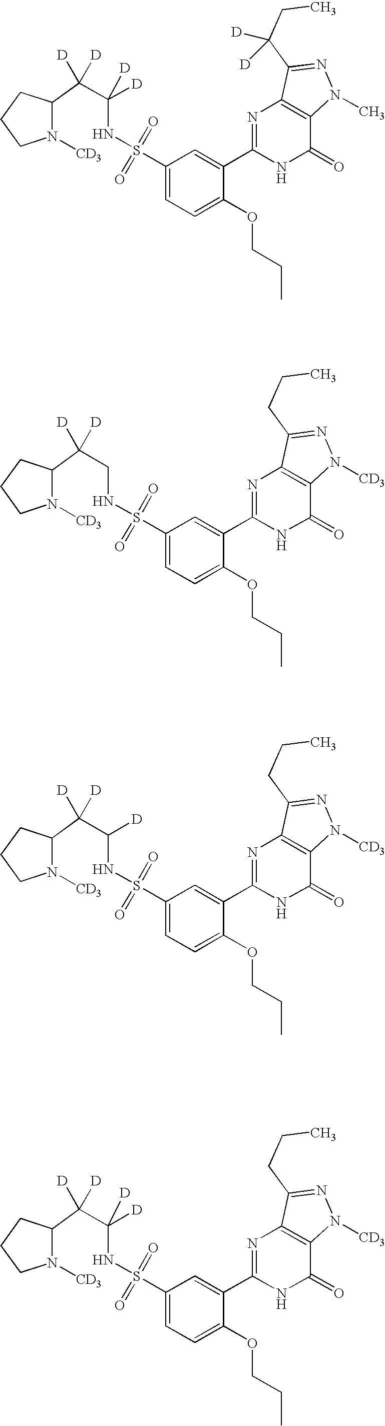

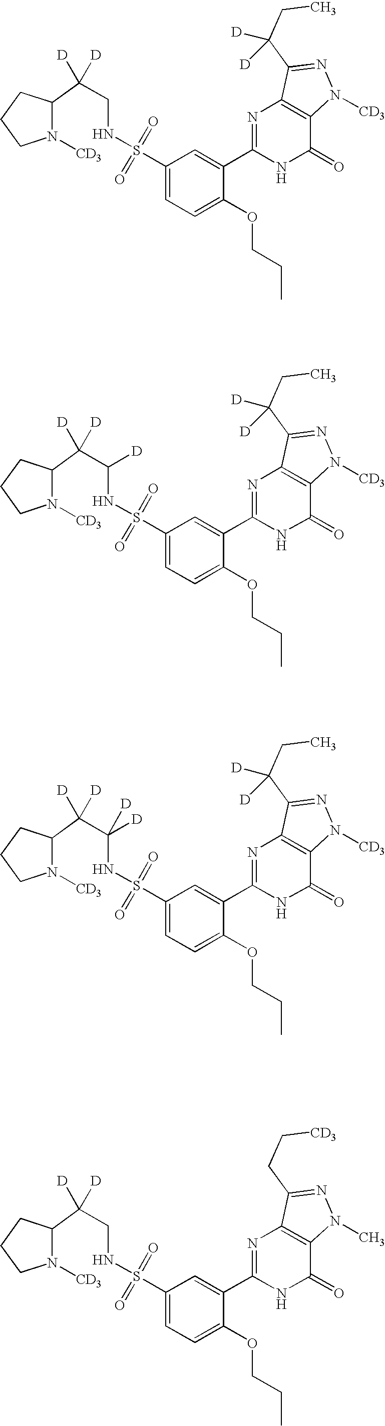

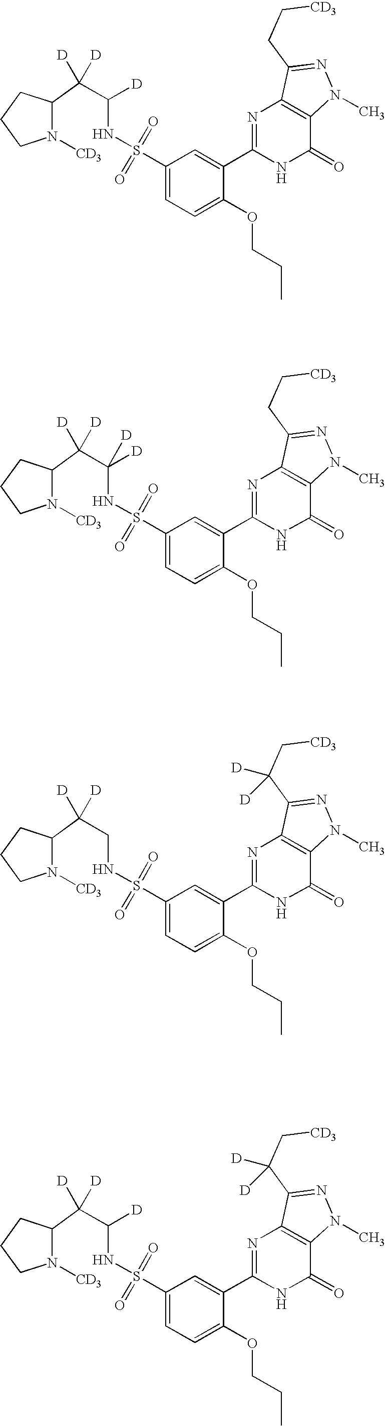

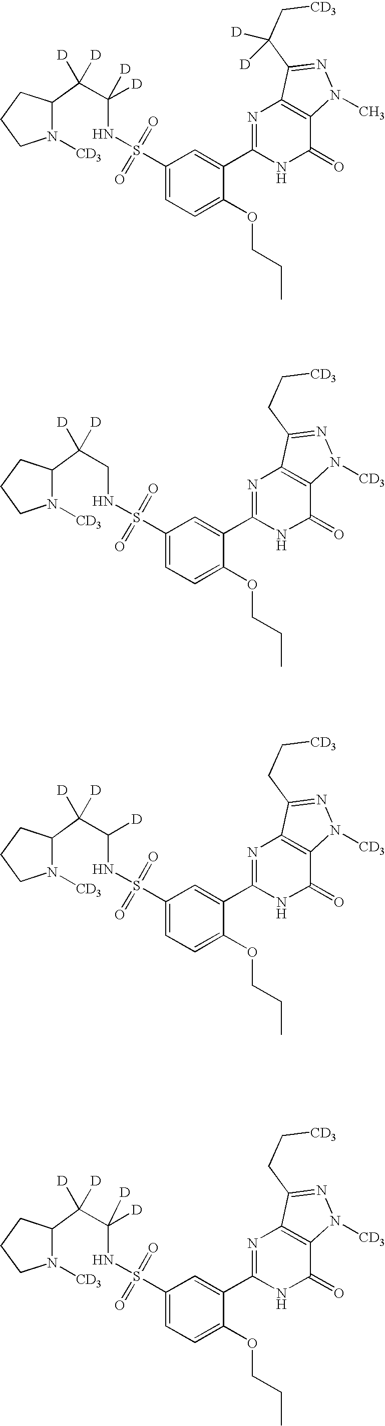

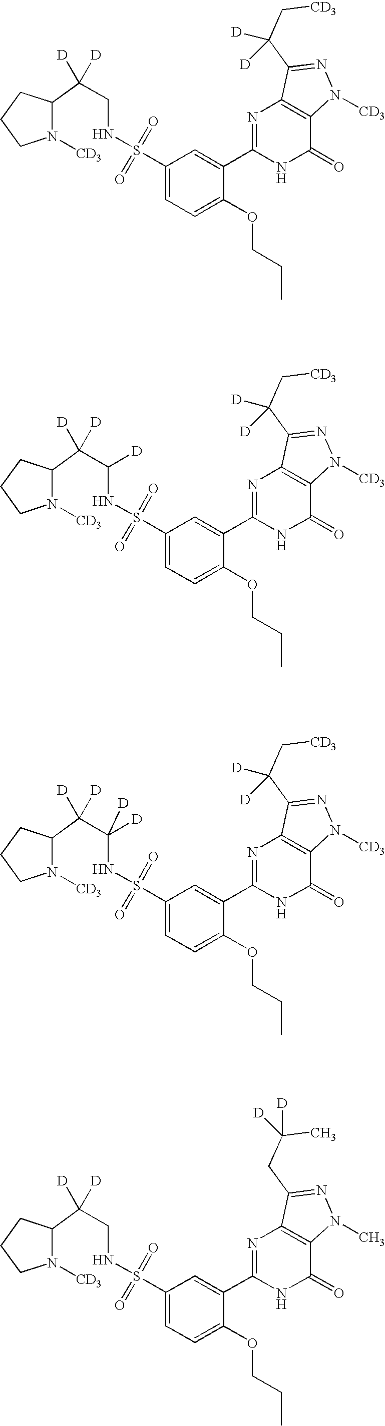

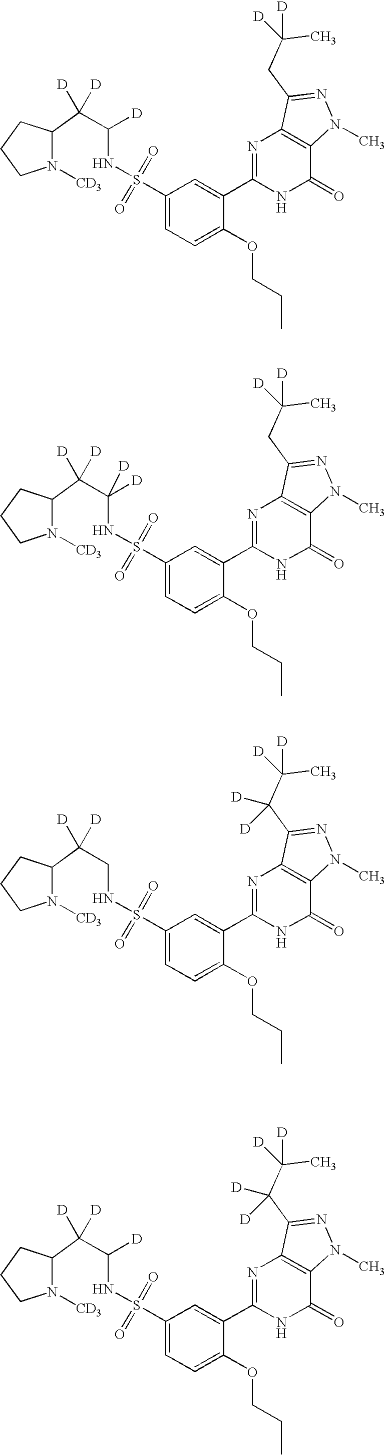

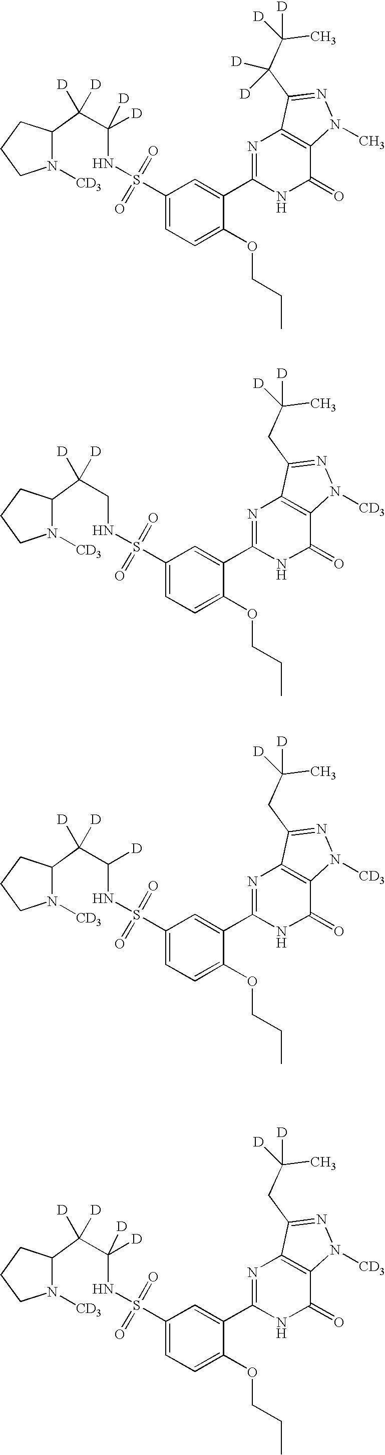

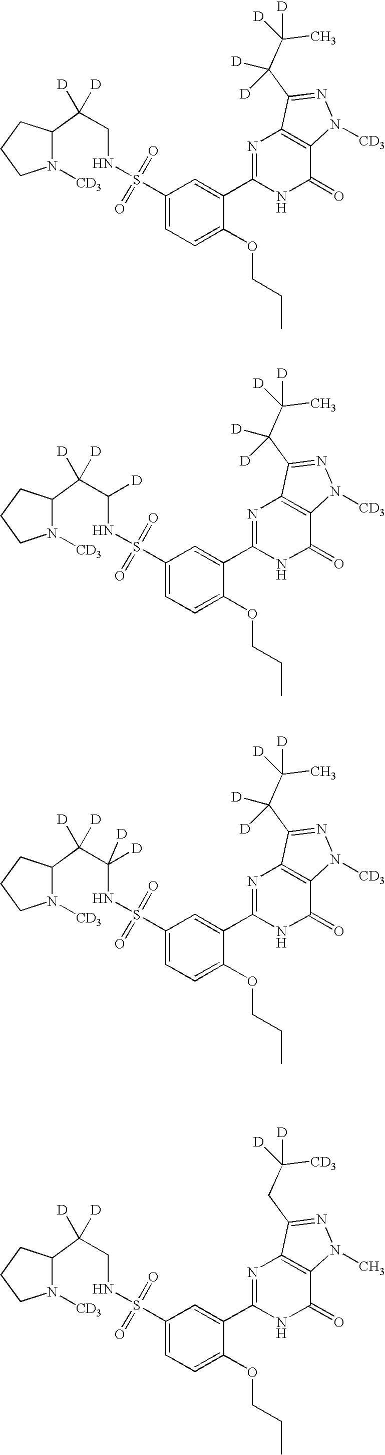

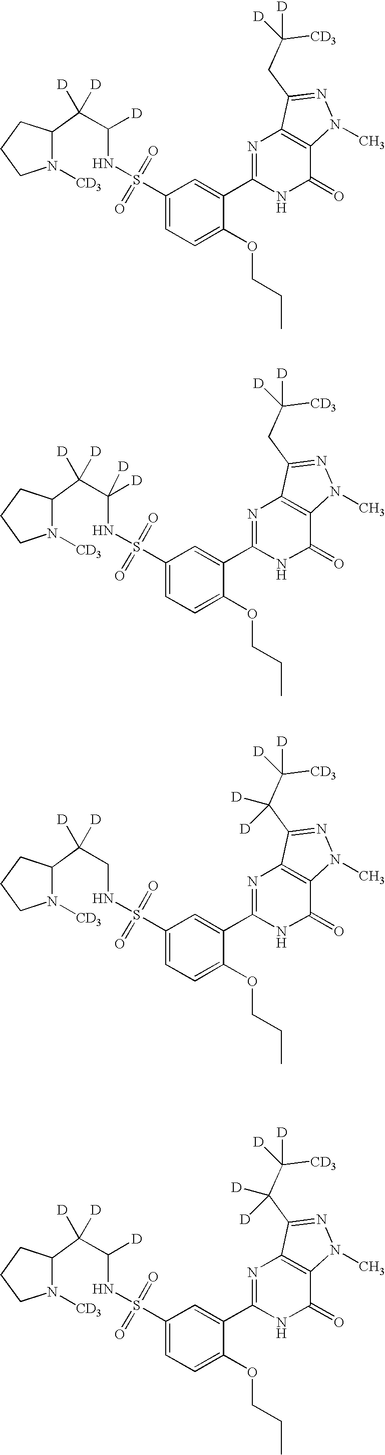

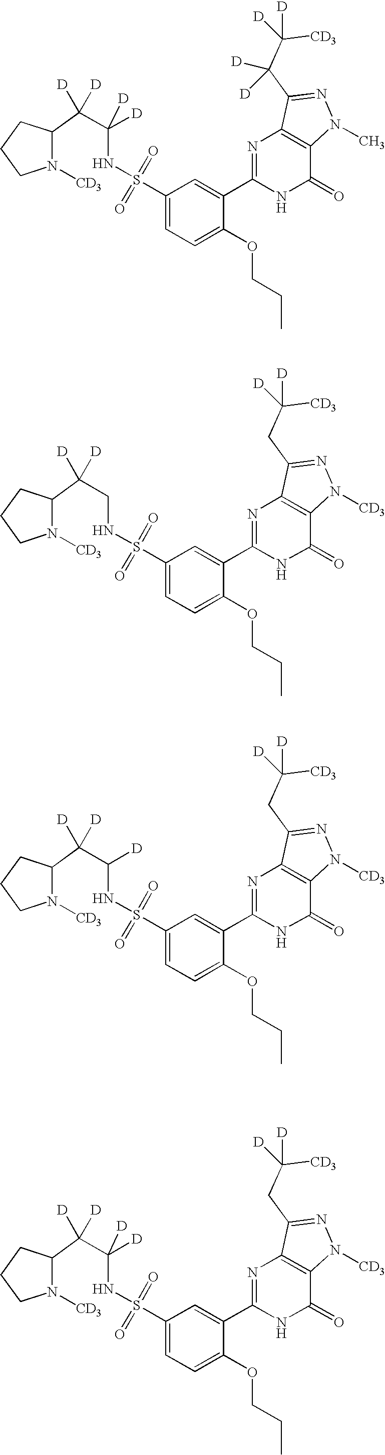

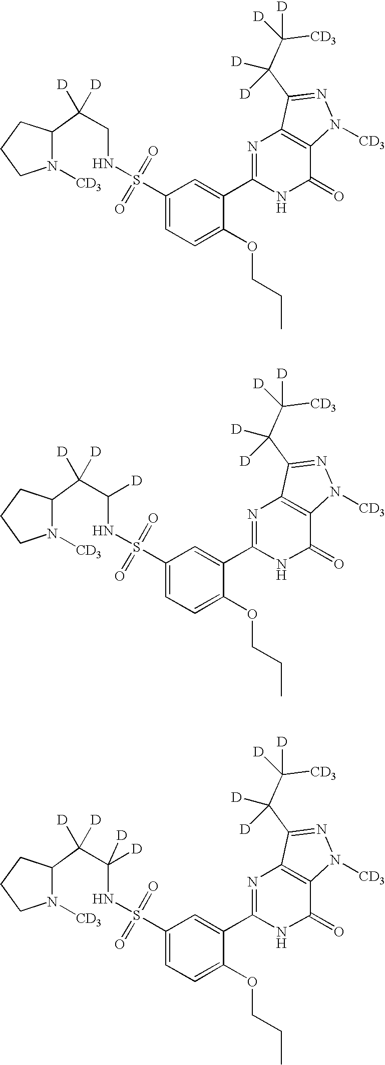

11. The compound as recited in claim 10 wherein said compound is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

12. The compound as recited in claim 10, wherein each of said positions represented as D have deuterium enrichment of at least 1%.

13. The compound as recited in claim 10, wherein each of said positions represented as D have deuterium enrichment of at least 5%.

14. The compound as recited in claim 10, wherein each of said positions represented as D have deuterium enrichment of at least 10%.

15. The compound as recited in claim 10, wherein each of said positions represented as D have deuterium enrichment of at least 20%.

16. The compound as recited in claim 10, wherein each of said positions represented as D have deuterium enrichment of at least 50%.

17. The compound as recited in claim 10, wherein each of said positions represented as D have deuterium enrichment of at least 90%.

18. The compound as recited in claim 10, wherein each of said positions represented as D have deuterium enrichment of at least 98%.

19. A pharmaceutical composition comprising a pharmaceutically acceptable carrier together with the compound as recited in claim 1.

20. The pharmaceutical composition of claim 19, wherein said composition is suitable for oral, parenteral, or intravenous infusion administration.

21. The pharmaceutical composition of claim 20, wherein said composition comprises a tablet, or capsule.

22. The pharmaceutical composition of claim 20, wherein said compound is administered in a dose of 0.5 milligram to 1000 milligram.

23. A pharmaceutical composition of claim 19, further comprising another therapeutic agent.

24. The pharmaceutical composition according to claim 23, wherein the therapeutic agent is selected from the group consisting of: phosphodiesterase 5 inhibitors, experimental erectile dysfunction treatments, CYP3A inhibitors, CYP3A inducers, protease inhibitors, antifungal agents, antibacterials, antimycobacterial agents, sepsis treatments, steroidal drugs, anticoagulants, thrombolytics, non-steroidal anti-inflammatory agents, antiplatelet agents, endothelin converting enzyme (ECE) inhibitors, thromboxane enzyme antagonists, potassium channel openers, thrombin inhibitors, growth factor inhibitors, platelet activating factor (PAF) antagonists, anti-platelet agents, Factor VIIa Inhibitors, Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-PDE5 agents, beta-PDE5 agents, antiarrhythmic agents, diuretics, anti-diabetic agents, PPAR-gamma agonists, mineralocorticoid enzyme antagonists, aP2 inhibitors, protein tyrosine kinase inhibitors, antiinflammatories, antiproliferatives, chemotherapeutic agents, immunosuppressants, anticancer agents, cytotoxic agents, antimetabolites, farnesyl-protein transferase inhibitors, hormonal agents, microtubule-disruptor agents, microtubule-stabilizing agents, topoisomerase inhibitors, prenyl-protein transferase inhibitors, cyclosporins, TNF-alpha inhibitors, cyclooxygenase-2 (COX-2) inhibitors, gold compounds, and platinum coordination complexes.

25. The pharmaceutical composition according to claim 24, wherein the therapeutic agent is a phosphodiesterase 5 inhibitor.

26. The pharmaceutical composition according to claim 24, wherein the therapeutic agent is an experimental erectile dysfunction treatment.

27. A pharmaceutical composition comprising a pharmaceutically acceptable carrier together with the compound as recited in claim 10.

28. The pharmaceutical composition of claim 27, wherein said composition is suitable for oral, parenteral, or intravenous infusion administration.

29. The pharmaceutical composition of claim 28, wherein said composition comprises a tablet, or capsule.

30. The pharmaceutical composition of claim 28, wherein said compound is administered in a dose of 0.5 milligram to 500 milligrams.

31. A pharmaceutical composition of claim 27, further comprising another therapeutic agent.

32. The pharmaceutical composition according to claim 31, wherein the therapeutic agent is selected from the group consisting of: phosphodiesterase 5 inhibitors, experimental erectile dysfunction treatments, CYP3A inhibitors, CYP3A inducers, protease inhibitors, antifungal agents, antibacterials, antimycobacterial agents, sepsis treatments, steroidal drugs, anticoagulants, thrombolytics, non-steroidal anti-inflammatory agents, antiplatelet agents, endothelin converting enzyme (ECE) inhibitors, thromboxane enzyme antagonists, potassium channel openers, thrombin inhibitors, growth factor inhibitors, platelet activating factor (PAF) antagonists, anti-platelet agents, Factor VIIa Inhibitors, Factor Xa Inhibitors, renin inhibitors, neutral endopeptidase (NEP) inhibitors, vasopepsidase inhibitors, HMG CoA reductase inhibitors, squalene synthetase inhibitors, fibrates, bile acid sequestrants, anti-atherosclerotic agents, MTP Inhibitors, calcium channel blockers, potassium channel activators, alpha-PDE5 agents, beta-PDE5 agents, antiarrhythmic agents, diuretics, anti-diabetic agents, PPAR-gamma agonists, mineralocorticoid enzyme antagonists, aP2 inhibitors, protein tyrosine kinase inhibitors, antiinflammatories, antiproliferatives, chemotherapeutic agents, immunosuppressants, anticancer agents, cytotoxic agents, antimetabolites, farnesyl-protein transferase inhibitors, hormonal agents, microtubule-disruptor agents, microtubule-stabilizing agents, topoisomerase inhibitors, prenyl-protein transferase inhibitors, cyclosporins, TNF-alpha inhibitors, cyclooxygenase-2 (COX-2) inhibitors, gold compounds, and platinum coordination complexes.

33. The pharmaceutical composition according to claim 32, wherein the therapeutic agent is a phosphodiesterase 5 inhibitor.

34. The pharmaceutical composition according to claim 32, wherein the therapeutic agent is an experimental erectile dysfunction treatment.

35. A method of treating a subject suffering from a PDE5-mediated disorder, comprising administering to said subject a therapeutically effective amount of a compound as recited in claim 1.

36. The method of claim 35 wherein said PDE5-mediated disorder can be ameliorated by administering a PDE5 enzyme modulator.

37. The method of claim 35, wherein said disorder is selected from the group consisting of hypertension, erectile dysfunction, and the inability to maintain improved erectile function

38. The method of claim 36, wherein said disorder is erectile dysfunction.

39. The method of claim 35, wherein said compound has at least one of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

40. The method of claim 35, wherein said compound has at least two of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

41. The method of claim 35, wherein said compound has a decreased metabolism by at least one polymorphically-expressed cytochrome P₄₅₀ isoform in said subject per dosage unit thereof as compared to the non-isotopically enriched compound.

42. The method of claim 41, wherein said cytochrome P₄₅₀ isoform is selected from the group consisting of CYP2C8, CYP2C9, CYP2C19, and CYP2D6.

43. The method of claim 35, wherein said compound is characterized by decreased inhibition of at least one cytochrome P₄₅₀ or monoamine oxidase isoform in said subject per dosage unit thereof as compared to the non-isotopically enriched compound.

44. The method of claim 43, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4B1, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

45. A method of treating a subject suffering from a PDE5-mediated disorder, comprising administering to said subject a therapeutically effective amount of a compound as recited in claim 10.

46. The method of claim 45 wherein said PDE5-mediated disorder can be ameliorated by administering a PDE5 enzyme modulator.

47. The method of claim 45, wherein said disorder is selected from the group consisting of hypertension, erectile dysfunction, and the inability to maintain improved erectile function

48. The method of claim 47, wherein said disorder is erectile dysfunction.

49. The method of claim 45, wherein said compound has at least one of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

50. The method of claim 45, wherein said compound has at least two of the following properties:
a) decreased inter-individual variation in plasma levels of said compound or a metabolite thereof as compared to the non-isotopically enriched compound;
b) increased average plasma levels of said compound per dosage unit thereof as compared to the non-isotopically enriched compound;
c) decreased average plasma levels of at least one metabolite of said compound per dosage unit thereof as compared to the non-isotopically enriched compound;
d) increased average plasma levels of at least one metabolite of said compound per dosage unit thereof as compared to the non-isotopically enriched compound; and
e) an improved clinical effect during the treatment in said subject per dosage unit thereof as compared to the non-isotopically enriched compound.

51. The method of claim 45, wherein said compound has a decreased metabolism by at least one polymorphically-expressed cytochrome P₄₅₀ isoform in said subject per dosage unit thereof as compared to the non-isotopically enriched compound.

52. The method of claim 51, wherein said cytochrome P₄₅₀ isoform is selected from the group consisting of CYP2C8, CYP2C9, CYP2C19, and CYP2D6.

53. The method of claim 45, wherein said compound is characterized by decreased inhibition of at least one cytochrome P₄₅₀ or monoamine oxidase isoform in said subject per dosage unit thereof as compared to the non-isotopically enriched compound.

54. The method of claim 53, wherein said cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.
